(19) United States
(12) Patent Application Publication
(10) Pub. No.: US 2025/0320190 A1
(43) Pub. Date: Oct. 16, 2025

(54) Novel Inhibitors of Leucine Carboxyl Methyltransferase-1 (LCMT-1) and Methods of Use Thereof

(71) Applicant: Temple University-Of The Commonwealth System of Higher Education, Philadelphia, PA (US)

(72) Inventors: ONEIDA AROSARENA, Huntingdon Valley, PA (US); Abdullah Saribas, Philadelphia, PA (US)

(21) Appl. No.: 19/094,075

(22) Filed: Mar. 28, 2025

Related U.S. Application Data
(60) Provisional application No. 63/571,533, filed on Mar. 29, 2024.

(51) Int. Cl. C07D 333/38; A61K 31/381; A61K 45/06; A61P 35/00

(52) U.S. Cl. CPC C07D 333/38; A61K 31/381; A61K 45/06; A61P 35/00

(57) ABSTRACT

The present disclosure relates to compounds and methods for inhibiting Leucine Carboxyl Methyltransferase-1 (LCMT-1), the methods comprising administering to the subject a compound of Formula (I). In some embodiments, the disclosure provides compounds and methods for treating cancer.

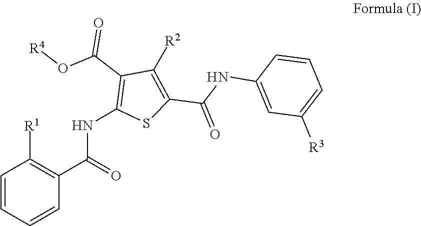

### CROSS REFERENCE TO RELATED APPLICATIONS

This application claims priority to U.S. Provisional Application No. 63/571,533, filed Mar. 29, 2024, which is hereby incorporated by reference herein in its entirety.

### STATEMENT OF GOVERNMENT SUPPORTED RESEARCH

This invention was made with government support under R21 CA167126 awarded by the National Institutes of Health. The government has certain rights in the invention.

### BACKGROUND OF THE INVENTION

Reversible phosphorylation is the basis for signal transduction in eukaryotic cells, and this is tightly controlled by the complex interplay of kinases and phosphatases. Protein phosphorylation is one of the most common post-translational modifications, altering structural conformation and function. It is necessary for a variety of cellular functions including mitosis, cell death, metabolism, organelle trafficking, differentiation and migration (Heo et al. Front Cell Neurosci. 2022; 16:852245; Cervone et al. Oncotarget. 2018; 9(7):7312-21; O'Connor et al. The international journal of biochemistry & cell biology. 2018; 96:182-93). Aberrant phosphorylation is a mark of disease states including cancer, diabetes and neurodegenerative diseases.

It is estimated that two-thirds of proteins encoded by the human genome undergo phosphorylation (Ardito et al. International journal of molecular medicine. 2017; 40(2):271-80), and there are 538 known human kinases (Zhang et al. Elife. 2021; 10). More than two-thirds of phosphorylation events occur on serine, threonine or tyrosine residues. Serine phosphorylation is the most common event, followed by threonine phosphorylation, with tyrosine phosphorylation being the rarest. In contrast to the kinases, there are approximately 200 known phosphatases, the majority of which are tyrosine phosphatases (Sacco et al. FEBS letters. 2012; 586(17):2732-9). Of the two families of serine/threonine phosphatases, the protein phosphatase-2A family (PP2A) is known to regulate development, apoptosis, transcription, translation, growth and cell division (Moura M, et al. Biomolecules. 2019; 9(2); Vaneynde et al. Front Cell Dev Biol. 2022; 10:1030119).

Many malignancies are characterized by dysregulation of the delicate protein phosphorylation balance due to mutations, chromosomal rearrangements or epigenetic modifications resulting in constitutive kinase activation (Cicenas et al. Cancers (Basel). 2018; 10(3). Much more is known about the kinases than the phosphatases, and several kinase inhibitors have been successful in the treatment of some malignancies. However, these successes have been limited by the development of drug resistance. The targeting of protein phosphatases has been gaining attention as their role in cancer development and progression has been elucidated (Haesen et al. Frontiers in oncology. 2014; 4:347; Turdo et al. Front Cell Dev Biol. 2021; 9:690306; Remmerie et al. Frontiers in oncology. 2019; 9:462; Xiao et al. Cell. 2018; 173(2):470-84 e18; Vainonen et al. Sci Transl Med. 2021; 13(588); Dai et al. Oncotarget. 2017; 8(56):95810-23; Sun et al. Cell Death Dis. 2021; 12(9):849; Uddin et al. MH, Cell Cycle. 2020; 19(5):592-600; D'Arcy et al. Oncotarget. 2019; 10(61):6543-5). LB-100, a PP2A inhibitor, is currently in clinical trial (Chung et al. Clinical cancer research: an official journal of the American Association for Cancer Research. 2017; 23(13):3277-84; Ronk et al. Cancer Biol Med. 2022; 19(10):1428-39).

PP2A enzymes have a heterotrimeric structure consisting of scaffolding (A), catalytic (C) and regulatory (B) subunits. While there are two isoforms each of the A and C subunits, substrate specificity is mediated primarily by the 23 isoforms of the B subunits (Vaneynde et al. Front Cell Dev Biol. 2022; 10:1030119; Haesen et al. Frontiers in oncology. 2014; 4:347). PP2A heterotrimers containing the B55α (PR55α) regulatory subunit have been associated with oncogenic signaling (Hein et al. Oncogenesis. 2019; 8(11):63; Hein et al. Cancer research. 2016; 76(8):2243-53; Smits et al. The EMBO journal. 1992; 11(12):4601-6; Di Conza et al. Cell Rep. 2017; 18(12):2836-44), and B55 subunits are found exclusively in forms of PP2A in which the carboxyl terminus of the catalytic subunit (PP2Ac) is methylated at leucine 309 (Longin et al. The Journal of biological chemistry. 2007; 282(37):26971-80).

Methylation of PP2Ac is controlled by two enzymes, a methyl transferase (leucine carboxyl methyltransferase-1 (LCMT-1)), which adds the methyl group with S-adenosylmethionine (SAM) as the methyl donor; and an esterase (PP2A methyl esterase (PME-1)), which removes the methyl group.

Thus, there is a need in the art for improved compositions and methods for inhibiting LCMT-1 and treating diseases associated with its overactivity. This invention satisfies this unmet need.

### SUMMARY OF THE INVENTION

In one aspect, the present invention relates to a compound of Formula (I), or a derivative, prodrug, or pharmaceutically acceptable salt or solvate thereof:

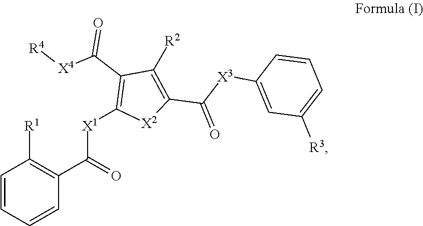

wherein: R¹, R², R³, and R⁴ are each independently selected from the group consisting of CH₃, CD₃, and CF₃; and X¹, X², X³, and X⁴ are each independently selected from the group consisting of O, NR^{X}, and CR^{X}₂, and each instance of R^{X} is independently selected from the group consisting of H, D, F, CH₃, CD₃, and CF₃.

In some embodiments, X¹ is NH. In some embodiments, X² is S. In some embodiments, X³ is NH. In some embodiments X⁴ is O. In some embodiments, at least one of R¹, R², R³, and R⁴ is CH₃. In some embodiments, R¹, R², R³, and R⁴ are all CH₃.

In some embodiments, the compound Formula (I) is a compound of Formula (II):

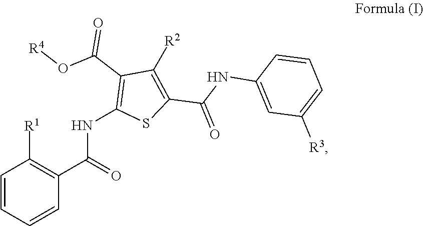

wherein R¹, R², R³, and R⁴ are each independently selected from the group consisting of CH₃, CD₃, and CF₃.

In some embodiments, at least one of R¹, R², R³, and R⁴ is CH₃. In some embodiments, R¹, R², R³, and R⁴ are all CH₃.

In another aspect, the present invention relates to a method of decreasing leucine carboxyl methyltransferase-1 (LCMT-1) activity in a subject comprising administering to the subject the claimed compounds.

In another aspect, the present invention relates to a method of treating a disease or disorder in a subject comprising administering to the subject the claimed compounds.

In some embodiments, the disease or disorder is cancer. In some embodiments, the cancer is selected from the group consisting of squamous cell carcinoma, melanoma, lung, leukemia, colorectal, breast, or pancreatic cancer.

In some embodiments, the compound of any one of claims 1-10 is administered in combination with one or more therapies selected from the group consisting of radiation therapy, surgery, chemotherapy, and immune checkpoint inhibitors. In some embodiments, the therapy is radiation therapy.

In some embodiments, the method comprises: a) administering to said subject the compound of any one of claims 1-10; and b) administering to said subject an effective amount of radiation therapy. In some embodiments, the radiation therapy is administered by way of a regimen selected from the group consisting of: 4-10 grays.

In one aspect, the present invention relates to a method of sensitizing a tumor in a subject to treatment comprising administering to the subject the claimed compound. In some embodiments, the method comprises sensitizing a tumor in a subject to radiation treatment.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1A through FIG. 1D depicts representative data demonstrating methylation of PP2A. FIG. 1A depicts a schematic diagram of PP2A assembly and activity. PP2A enzymes have a heterotrimeric structure consisting of one scaffolding (A) subunit (PR65), one catalytic (C) subunit and one regulatory (B) subunit. Owing to various isoforms of each of these subunits (two A, two C and 23 B isoforms), 92 different heterotrimeric complexes can be assembled, each with its own specific substrate. Methylation of the catalytic subunit of PP2A near its carboxyl terminus at leucine-309 facilitates docking of certain regulatory subunits, including the B55α subunit (PR55α). This methylation is catalyzed by LCMT-1, the primary cellular PP2A methyltransferase. In this reaction, SAM is the methyl donor. PP2Ac is demethylated by PME-1. FIG. 1B (left) depicts structure of SAM (generated with MolView software). FIG. 1B (center) depicts X-ray crystallographic model of LCMT-1 in complex with SAM. FIG. 1B (right) depicts X-ray crystallographic model of LCMT-1 (blue) in complex with SAM (pink) and PP2Ac (yellow). FIG. 1C (left) depicts the structure of Compound 1 (N-(2-Hydroxyphenyl)-2-(1-naphthal enyloxy)acetamide. FIG. 1C (center and right) depict X-ray crystallographic models of LCMT-1 (yellow) in complex with SAM (red) and Compound 1 (blue). Note that Compound 1 does not interact with LCMT-1 at the SAM binding site. FIG. 1D (left) depicts the structure of the Compound 2 (methyl 4-methyl-2-[(2-methylbenzoyl)amino]-5-[[(3-methylphenyl)amino]carbonyl]-3thiophenecarboxylate). FIG. 1D (right) depicts X-ray crystallographic models of LCMT-1 (yellow) in complex with Compound 2 (green) and SAM (red). Note that Compound 2 does not interact with LCMT-1 at the SAM binding site.

FIG. 2A through FIG. 2D depicts representative results of example experiments demonstrating that compound 2 inhibits LCMT-1-mediated methylation of PP2Ac. FIG. 2A depicts representative dot immunoblots demonstrating decreased methylation of rhPP2Ac by rhLCMT-1 in the presence of sinefungin and Compound 2. Bars represent means±standard errors of the mean (SEM). FIG. 2B depicts representative Western blot demonstrating demethylation of PP2Ac in MDA-MB-231 cells in the presence of Compound 2, but not in the presence of Compound 1 (means±SEM). FIG. 2C depicts representative Western blot demonstrating demethylation of PP2Ac in Rosi cells in the presence of Compound 2, but not in the presence of Compound 1 (means±SEM). FIG. 2D depicts the results of example experiments demonstrating neither Compound 1 nor Compound 2 significantly decreased PP2Ac methylation in MCF7 cells (means±SEM).

FIG. 3A through FIG. 3H depicts representative results of example experiments demonstrating compound 2 significantly inhibits survival of HEK-293, HS-5, MDA-MB-231, and Rosi cells, but not MCF7 cells and LCMT-1 overexpression confers resistance to compound 2-mediated inhibition of cell survival. FIG. 3A depicts the results of example experiments demonstrating compound 2 inhibited survival of HEK-293 cells at concentrations between 5 and 20 μM (p=0.0192 at 5 μM, p=0.0014 at 10 μM, and p=0.0003 at 20 μM), while LB-100 inhibited survival at 20 μM (p=0.0036). Data represent means±SEM. FIG. 3A depicts representative photomicrographs demonstrating cell death in cultures treated with LB-100 and necrotic figures in cultures treated with Compound 2. Compound 2 inhibited survival of Rosi cells at concentrations between 5 and 20 μM (p=0.0003 at 5 μM, p<0.0001 at 10 μM, and p<0.0001 at 20 μM), while LB-100 inhibited survival at 20 μM (p=0.0035). Note necrotic figures in cultures treated with Compound 2. Data represent means±SEM. FIG. 3C depicts the results of example experiments demonstrating that in HS-5 cells, Compound 2 cytotoxicity at 20 μM (p=0.0004) was comparable to that of LB-100 at 20 μM (p=0.0087). Data represent means±SEM. FIG. 3D depicts the results of example experiments demonstrating that in MDA-MB-231 cells, LB-100 inhibited survival in concentrations between 25 and 50 μM (p=0.0117 at 25 μM and p=0.0023 at 50 μM), while Compound 2 significantly inhibited survival at 25 μM (p=0.0425). Data represent means±SEM. FIG. 3E depicts the results of example experiments demonstrating neither LB-100 nor Compound 2 significantly inhibited survival in MCF7 cells at the concentrations tested (p=0.3204). Data represent means±SEM. FIG. 3F depicts baseline expression of LCMT-1 in cell lines. Relative overexpression of LCMT-1 is demonstrated in HEK-293 and MCF7 cells. FIG. 3g depicts the result of example experiments wherein HEK-293 cells were transfected with a LCMT-1 mammalian expression vector (left). After 48 hours, cells were treated with Compound 2. Overexpression of LCMT-1 in HEK-293 cells enhanced resistance to Compound 2-mediated cell death (right). FIG. 3H depicts the results of example experiments wherein MDA-MB-231 cells were transfected with a LCMT-1 mammalian expression vector (left). After 48 hours, cells were treated with Compound 2. Overexpression of LCMT-1 in MDA-MB-231 induced resistance to Compound 2-mediated cell death (right).

FIG. 4A through FIG. 4C depicts representative results of example experiments demonstrating that compound 2 inhibits clonogenic colony formation in cancer cells. FIG. 4A depicts the results of example experiments demonstrating LB-100 and Compound 2 inhibit colony formation in MDA-MB-231 cells in a concentration-dependent manner. FIG. 4B depicts the results of example experiments demonstrating LB-100 and Compound 2 inhibit colony formation in Rosi cells. FIG. 4C depicts the results of example experiments demonstrating LB-100 and Compound 2 inhibit colony formation in MCF7 cells. Data represent means±SEM.

FIG. 5A through FIG. 5C depicts representative results of example experiments demonstrating that compound 2 inhibits mitosis in Rosi cells. FIG. 5A depicts representative BrdU cell cycle analyses demonstrating that LB-100 induces G2/M-phase arrest in MDA-MB-231 cells. Compound 2 did not change cell cycle dynamics in this cell line. FIG. 5B depicts the results of example experiments demonstrating LB-100 induces G2/M-phase arrest in Rosi cells, while Compound 2 induced G0/G1 arrest. FIG. 5C depicts the results of example experiments demonstrating LB-100 induces G0/G1 arrest in MCF7 cells. Compound 2 did not change cell cycle dynamics in this cell line. Data represent means±SEM.

FIG. 6A through FIG. 6C depicts representative results of example experiments demonstrating that compound 2 induces apoptosis in MDA-MB-231 cells, but not MCF7 nor Rosi cells. FIG. 6A depicts a representative histogram demonstrating that Compound 2 decreases cell surface annexin V expression in MCF7 cells. FIG. 6B depicts the results of example experiments demonstrating Both LB-100 and Compound 2 induced apoptosis in MDA-MB-231 cells. FIG. 6C depicts the results of example experiments demonstrating LB-100, but not Compound 2, induces apoptosis in Rosi cells. Data represent means±SEM.

FIG. 7A through FIG. 7C depicts representative results of example experiments demonstrating that compound 2 induces RIPK1 phosphorylation in cancer cells. FIG. 7A depicts representative Western blots demonstrating that treatment with Compound 2 resulted in decreased cytosolic expression of PP2Ab in MCF7 cells beginning at 4 hours. Cytosolic PP2Ab expression recovered after 6 hours. Treatment with Compound 2 resulted in increased phosphorylation of RIPK1, MLKL, AMPKα and ACC1 in MCF7 cells. FIG. 7B depicts representative Western blots demonstrating that treatment with Compound 2 decreased cytosolic expression of PP2Ab at 6 hours, with recovery of cytosolic PP2Ab at 8 hours. Compound 2 increased phosphorylation of RIPK1 in Rosi cells (p=0.0179) and MLKL. Increased phosphorylation of AMPKα and ACC1 was not statistically significant. FIG. 7C depicts representative Western blots demonstrating that treatment with Compound 2 did not change cytosolic expression PP2Ab or phosphorylation of pAMK in MDA-MB-231 cells. Compound 2 increased phosphorylation of RIPK1 (p=0.0273) and MLKL. Data represent means±SEM.

FIG. 8 depicts a representative radioactive LCMT-1 assay. Recombinant human LCMT-1 (rhLCMT-1) was incubated with the rhPP2A-A (scaffolding unit)/rhPP2A-C(catalytic subunit) heterodimer and tritiated SAM for one hour at room temperature in 50 μL reactions at 20 μM concentrations of each component in 50 mM TrisHCl, pH 7.2 with 0.5 mM DTT. For reactions with LCMT-1 inhibitors, the inhibitors were added at a concentration of 1 mM. The reactions were quenched with 150 μL 10% trichloroacetic acid (TCA). The reactions were transferred to 96-well filter plates. The filters were dried with a vacuum, then washed thrice with 200 μL 10% TCA. The filters were dried again, and 50 μL BetaPlate Scint fluid (Perkin Elmer) was added to each well. Incorporated radioactivity was measured with a MicroBeta (Perkin Elmer) scintillation counter.

FIG. 9A through FIG. 9C depicts representative kinetics of PP2A methylation by LCMT-1 as measured by radioactive LCMT-1 assays. FIG. 9A depicts a Michaelis-Menten curve demonstrating titration of SAM in LCMT-1 activity assay. FIG. 9B depicts a Michaelis-Menten curve demonstrating titration of PP2AC heterodimer in LCMT-1 activity assay. FIG. 9C depicts the results of example experiments demonstrating titration of LCMT-1 reveals a linear relationship with respect to PP2AC. Error bars indicate standard deviations, n=3.

FIG. 10 depicts representative results of example experiments demonstrating that methylation of PP2AC by LCMT-1 reaches maximum by 20 minutes at room temperature. Error bars indicate standard deviations, n=3.

FIG. 11 depicts representative kinetics of sinefungin (SAM analogue) inhibition LCMT-1 methylation of the rhPP2A-A (scaffolding unit)/rhPP2A-C(catalytic subunit) heterodimer in the presence of SAM. Error bars indicate standard deviations, n=3.

FIG. 12 depicts a representative radioactive LCMT-1 assay. SF inhibition indicates inhibition by sinefungin.

FIG. 13 depicts representative kinetics of LCMT-1 inhibition by compound 1 (in red) and compound 2 (in green). Error bars indicate standard deviations, n=3.

FIG. 14 depicts representative results of example experiments wherein a panel of cell lines were screened for sensitivity to PR55a depletion using glucose starvation. SCC-15 and SCC-25 cells were seeded in 96-well plates at a density of 1.6×104 cells/well in Dulbecco's modified Eagle's medium with Ham's F-12 (DMEM/F-12, Corning®, Glendale, Arizona, catalog #90-090-PB) with 10% fetal bovine serum (FBS, Corning® catalog #35-015-CV). The remaining cells were plated at the same density in DMEM (Corning® catalog #50-003-PC) with 10% FBS. Cells were maintained in a 5% carbon dioxide atmosphere at 37° C. and tested free of mycoplasma contamination. After 24 hours, the medium was changed to glucose-free DMEM (Gibco Thermo-Fisher Scientific, Waltham, Massachusetts, catalog #A14430-01) with 10% FBS. After 24 hours cells were stained with propidium iodide (Cayman Chemical Company, Ann Arbor, Michigan, catalog #10008351). Photomicrographs of each well were taken with 4× magnification. Stained cells were counted with National Institutes of Health ImageJ software. N=4 for each condition.

FIG. 15 depicts (left) a representative model demonstrating binding of SAM (red) with ARMT1 α chain. (right) A model demonstrating possible interaction of compound 2 (green) with ARMT1 α chain. Compound 2 is binding in the same location as SAM according to the model.

FIG. 16 depicts (left) a representative model demonstrating binding of SAM (red) with ARMT1 β chain. (right) A model demonstrating possible interaction of compound 2 (green) with ARMT1β chain. Compound 2 does not bind in the same location as SAM according to the model.

FIG. 17 depicts (left) a representative model demonstrating binding of SAM (red) with MLL5 SET domain. (right) A model demonstrating possible interaction of compound 2 (green) with MLL5 SET domain. Compound 2 binds in the same location as SAM according to the model.

FIG. 18A through FIG. 18M depicts representative results of example experiments demonstrating that LCMT-li radiosensitizes cell lines. Cells were treated with various doses of LCMT-li or with the carrier (DMSO) for 3 hours and then irradiated. Control cells received a medium change. LCMT-li significantly radio-sensitized SCC-15 (FIG. 18A), SCC-25 (FIG. 18B), UM-SCC-12 (FIG. 18C), UM-SCC-14a (FIG. 18D), UM-SCC-49 (FIG. 18E) and UM-SCC-69 (FIG. 18F) cells to irradiation (top panel). IR indicates irradiation. Graphs indicate means±standard errors of the mean and so throughout. Bottom panel (FIG. 18G) has representative images of crystal violet-stained cells, 4× magnification. LCMT-li significantly radio-sensitized 501MEL (FIG. 18H), A549 (FIG. 18I), MCF7 (FIG. 18K) and THP-1 (FIG. 18L) cells, but not Jurkat cells (FIG. 18J, top panel). Bottom panel has representative images of MCF7, MDA-MB-231 and Rosi crystal violet-stained cells (FIG. 18M).

FIG. 19A through FIG. 19F depicts representative results of example experiments demonstrating LCMT-li effects on cell death alone and with irradiation. FIG. 19A depicts data showing LCMT-li alone and in combination with irradiation increased apoptosis in SCC-15 cells (p<0.001 for both comparisons). LCMT-li did not induce necroptosis or ferroptosis alone or with irradiation. FIG. 19B depicts data showing that SCC25 cells responded to irradiation primarily with necrosis. LCMT-li did not increase apoptosis or ferroptosis alone or in combination with irradiation. LCMT-li increased necroptosis alone and in combination with irradiation as evidenced by increased pMLKL expression. FIG. 19C depicts data showing LCMT-li alone increased apoptosis in UM-SCC-12 cells, but did not increase apoptosis in the presence of irradiation as compared to irradiation alone. LCMT-li alone increased necroptosis (p<0.0001) and ferroptosis (increased ACSL4) in these cells. FIG. 19D depicts data showing that LCMT-li combined with irradiation did not increase programmed cell death in UM-SCC-14a cells. FIG. 19E depicts data showing that like SCC-25 cells, UM-SCC-49 cells responded to irradiation primarily with necrosis. Apoptosis, necroptosis and ferroptosis did not differ between the experimental conditions. FIG. 19F depicts data showing that LCMT-li alone and with irradiation increased apoptosis in UM-SCC-69 cells.

FIG. 20A through FIG. 20F depicts representative results of example experiments demonstrating that LCMT-li has varied effects on the cell cycle with and without irradiation. FIG. 20A depicts data demonstrating that in the presence of irradiation, LCMT-li increased G0/G1 as well as G2/M phase arrest in SCC15 cells (p<0.0001 for both comparisons). SCC-25 (FIG. 20B) and UM-SCC-12 cells (FIG. 20C) demonstrated increased G2/M phase arrest with the LCMT-li/irradiation combination as compared to irradiation alone (p<0.0001 for SCC25, p=0.0204 for UM-SCC-12). In UM-SCC-14a, UM-SCC-49 and UM-SCC-69 cells (FIG. 20D-FIG. 20F) LCMT-li alone increased G0/G1 arrest as compared to control cells (p<0.0001 for UM-SCC-14a and UM-SCC-49 cells, p=0.0003 for UM-SCC-69 cells), but decreased G2/M phase arrest in the presence of irradiation as compared to irradiation alone (p=0.0038 for UM-SCC-14a, p<0.0001 for UM-SCC-49. and p=0.0047 for UM-SCC-69 cells).

FIG. 21A through FIG. 21F depicts representative results of example experiments demonstrating that LCMT-li impacts activated γ-H2AX expression with and without irradiation. LCMT-li alone increased activated γ-H2AX expression in SCC-25 as compared to control cells (FIG. 21B, p=0.0001); and decreased activated γ-H2AX expression in UM-SCC-12 (FIG. 21C), UM-SCC-14a (FIG. 21D), UM-SCC-49 (FIG. 21E) and UM-SCC-69 (FIG. 21F) cells (p<0.0001 for all comparisons). UM-SCC-14a (FIG. 21D) and UM-SCC-49 (FIG. 21E) cells expressed relatively high baseline activated γ-H2AX characteristic of cells with impaired DDR and high mutational burden (Ji, J. et al., 2017, PLoS One 12(2): e0171582). In the presence of irradiation, LCMT-li only inhibited DDR as evidenced by persistent activated γ-H2AX expression in UM-SCC-69 cells (p<0.0001).

### DETAILED DESCRIPTION OF THE INVENTION

In one aspect, the disclosure is based in part on the unexpected finding of novel compounds which inhibit leucine carboxyl methyltransferase-1 (LCMT-1). In some embodiments, these compounds are useful for treating diseases and/or disorders. For example, in some embodiments, the compounds of the disclosure may treat cancer.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, +10%, +5%, ±1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “therapeutic” treatment is a treatment administered to a subject who exhibits signs or symptoms of pathology disease or disorder, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “potency” refers to the dose needed to produce half the maximal response (ED₅₀).

As used herein, the term “efficacy” refers to the maximal effect (Eₘₐₓ) achieved within an assay.

As used herein, “activity” includes physiological activity, binding affinity, and/or the enzymatic activity of a molecule.

As used herein, “LCMT-1” refers to leucine carboxyl methyltransferase-1.

As used herein, “associated” refers to coincidence with the development or manifestation of a disease, condition, or phenotype. Association may be due to, but is not limited to, genes responsible for housekeeping functions, those that are part of a pathway that is involved in a specific disease, condition, or phenotype and those that indirectly contribute to the manifestation of a disease, condition or phenotype.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, colorectal cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds of the present invention.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of 0, N, and S, and wherein the nitrogen and sulfur atoms may be optionally oxidized, and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include —O—CH₂—CH₂—CH₃, —CH₂—CH₂—CH₂—OH, —CH₂—CH₂—NH—CH₃, —CH₂—S—CH₂—CH₃, —CH₂—CH₂—S(═O)—CH₃, and —CH₂—CH₂—S(═O)₂—CH₃. Up to two heteroatoms may be consecutive, such as, for example, —CH₂—NH—O—CH₃ or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e., skeletal atoms) is a carbon atom. In some embodiments, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties.

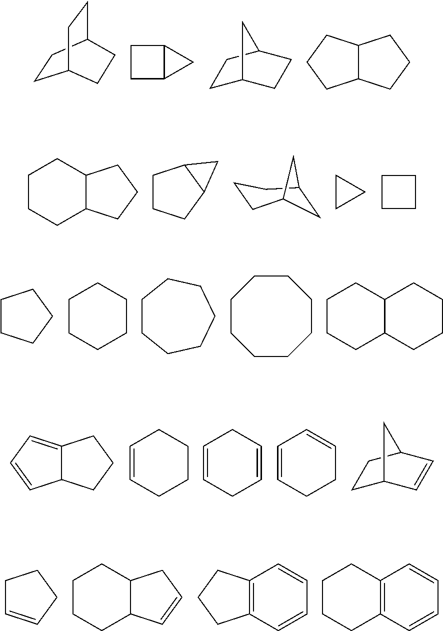

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In some embodiments, each heterocycloalkyl group has from 4 to 10 atoms in its ring system, with the proviso that the ring of said group does not contain two adjacent O or S atoms. In another embodiment, the heterocycloalkyl group is fused with an aromatic ring. In some embodiments, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In some embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

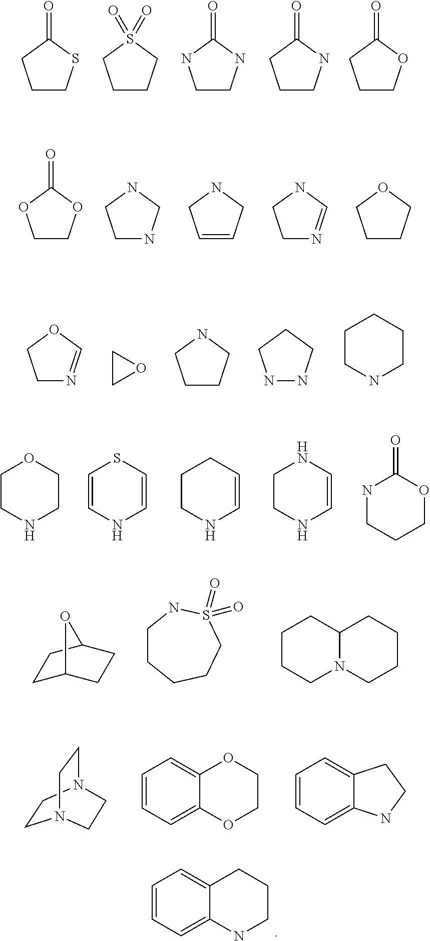

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. In some embodiments, aryl-(C₁-C₃)alkyl is aryl-CH₂— or aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

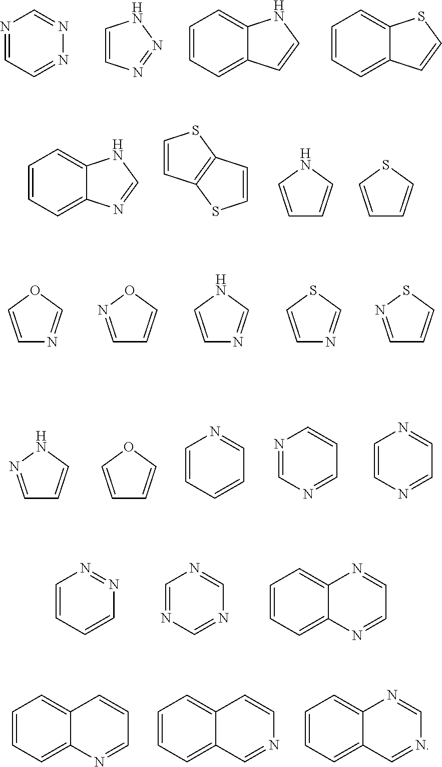

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In some embodiments, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In some embodiments, the referenced group is optionally substituted with zero substituents, i.e., the referenced group is unsubstituted. In another embodiment, the referenced group is optionally substituted with one or more additional group(s) individually and independently selected from groups described herein.

In some embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates generally to compositions and methods for inhibiting leucine carboxyl methyltransferase-1 (LCMT-1). In one embodiment, the present invention relates to a composition comprising one or more inhibitors of LCMT-1.

In some embodiments, the present invention relates to methods of treating cancer in a subject in need thereof.

### Compounds

The compounds of the present disclosure may be synthesized using techniques well-known in the art of organic synthesis. The starting materials and intermediates required for the synthesis may be obtained from commercial sources or synthesized according to methods known to those skilled in the art.

In one aspect, the disclosure provides compounds of Formula (I), or a derivative, prodrug, or pharmaceutically acceptable salt or solvate thereof:

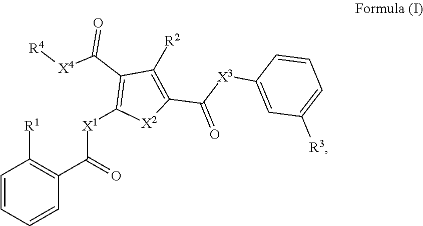

wherein:
   R¹, R², R³, and R⁴ are each independently selected from the group consisting of C₁-C₆ alkyl, C₃-C₆ branched alkyl, and C₃-C₆ cycloalkyl, wherein each of R¹, R², R³, and R⁴ is optionally substituted;
   X¹, X², X³, and X⁴ are each independently selected from the group consisting of O, NR^{X}, and CR^{X}₂,
   and each instance of R^{X} is independently selected from the group consisting of C₁-C₆ alkyl, C₃-C₆ branched alkyl, and C₃-C₆ cycloalkyl, wherein
each of R^{X} is optionally substituted.

In some embodiments, R¹, R², R³, and R⁴ are each independently selected from the group consisting of CH₃, CD₃, and CF₃.

In some embodiments, X¹, X², X³, and X⁴ are each independently selected from the group consisting of O, NR^{X}, and CR^{X}₂, wherein each instance of R^{X} is independently selected from the group consisting of C₁-C₆ alkyl, wherein each of R^{X} is optionally substituted.

In some embodiments, the disclosure provides compounds of Formula (I), or derivatives, prodrugs, or pharmaceutically acceptable salts or solvates thereof:

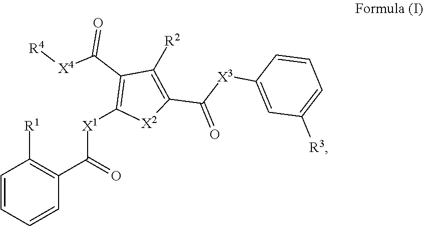

wherein:
R¹, R², R³, and R⁴ are each independently selected from the group consisting of CH₃, CD₃, and CF₃;
X¹, X², X³, and X⁴ are each independently selected from the group consisting of O, NR^{X}, and CR^{X}₂;
and each instance of R^{X} is independently selected from the group consisting of H, D, F, CH₃, CD₃, and CF₃.

In some embodiments, X¹ is NH. In some embodiments, X² is S. In some embodiments, X³ is NH. In some embodiments X⁴ is O. In some embodiments, at least one of R¹, R², R³, and R⁴ is CH₃. In some embodiments, R¹, R², R³, and R⁴ are all CH₃.

In some embodiments, the compound of Formula (I) is a compound of Formula (II):

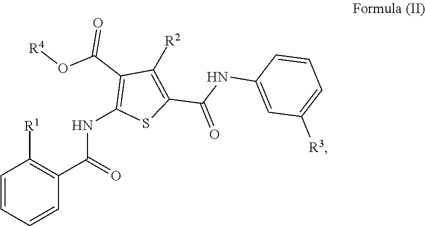

wherein R¹, R², R³, and R⁴ are each independently selected from the group consisting of CH₃, CD₃, and CF₃.

In some embodiments, at least one of R¹, R², R³, and R⁴ is CH₃. In some embodiments, R¹, R², R³, and R⁴ are CH₃.

The compounds of the invention may possess one or more stereocenters, and each stereocenter may exist independently in either the R or S configuration. In some embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In some embodiments, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The compounds described herein include the use of N oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In some embodiments, the compounds described herein exist in solvated forms with pharmaceutically acceptable solvents such as water, and ethanol. In another embodiment, the compounds described herein exist in unsolvated form.

In some embodiments, the compounds of the invention may exist as tautomers. All tautomers are included within the scope of the compounds presented herein.

In some embodiments, compounds described herein are prepared as prodrugs. A “prodrug” refers to an agent that is converted into the parent drug in vivo. In some embodiments, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In some embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In some embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to 2H, 3H, 11C, 13C, 14C, 36Cl, 18F, 123I, 125I, 13N, 15N, 15O, 17O, 18O, 32P, and 35S. In some embodiments, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as 11C, 18F, 15O and 13N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference in their entirety). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In some embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4 dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

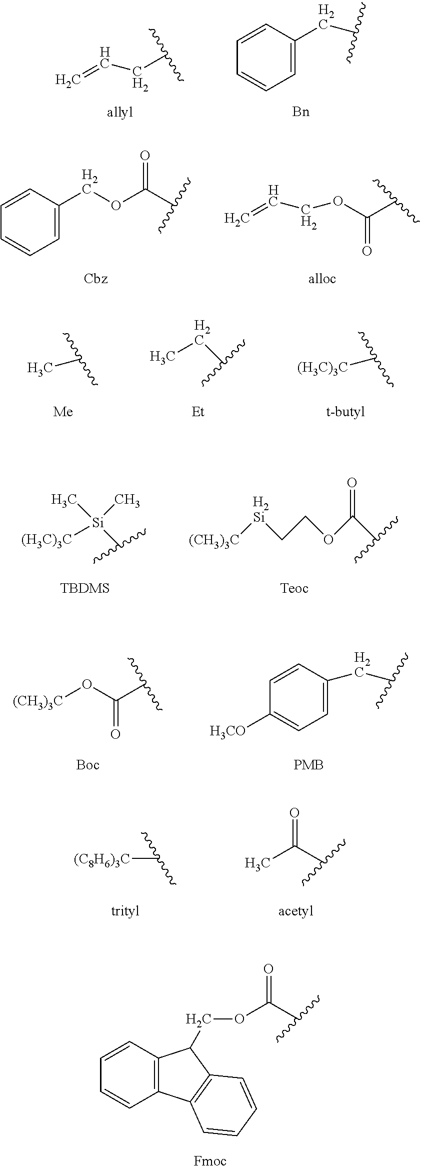

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods

In some embodiments, the disclosure provides methods of inhibiting LCMT-1 activity in a subject in need thereof. In some embodiments, the method comprises administering to the subject an effective amount of a composition comprising a compound of the disclosure. In some embodiments, the compound of the disclosure binds to LCMT-1 thereby inhibiting its activity.

In some embodiments, the disclosure provides methods comprising administering to a subject a compound of the disclosure. In some embodiments, the subject has a disease or disorder associated with LCMT-1 activity. In some embodiments, the disclosure provides a method of treating or preventing a disease or disorder associated with LCMT-1 activity. In some embodiments, the disease or disorder is cancer. In some embodiments, the cancer is selected from the group consisting of ovarian cancer, fallopian tube cancer, primary peritoneal cancer, endometrial cancer, uterine cancer, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma,hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, pancreatic, breast, colorectal, melanoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, carcinoma vilosum liposarcomas, leiomyosarcomas, rhabdomyosarcomas, neurofibrosarcomas, malignant peripheral nerve sheath tumors, Ewing's tumors, primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST), osteosarcoma (also known as osteogenic sarcoma) skeletal and extra-skeletal, chondrosarcoma, mantel cell lymphoma, follicular lymphoma, diffuse large B-cell lymphoma, T-cell acutelymphoblastic leukemia, Burkitt lymphoma, myeloma, immunocytoma, acute promyelocyte leukemia, chronic myeloid/acute lymphoblastic leukemia, acute leukemia, B-cell acute lymphoblastic leukemia, anaplastic large cell leukemia, myelodysplasia syndrome/acute myeloid leukemia, non-Hodgkin's lymphoma, chronic lymphocytic leukemia, acute myelogenous leukemia (AML), common (pre-B) acute lymphocytic leukemia, malignant melanoma, T-cell lymphoma, leukemia, B-cell lymphoma, epithelial malignancies, lymphoid malignancies, gynecologic carcinoma, pancreatic biliary adenocarcinoma, and pancreatic ductal adenocarcinomas. In some embodiments, the cancer is selected from the group consisting of squamous cell carcinoma, melanoma, lung, leukemia, colorectal, breast, or pancreatic cancer.

In some embodiments, the disclosure provides methods of treating cancer in a subject in need thereof. In some embodiments, the method comprises administering to the subject an effective amount of a compound of the disclosure.

In some embodiments, the method further comprises administering to the subject at least one additional therapy. In some embodiments, the therapy is selected from the group consisting of a selected from the group consisting of radiation therapy, surgery, chemotherapy, checkpoint inhibitors, and combinations thereof. In some embodiments, the therapy comprises administering the compound of any one of claims 1-4 in combination with radiation. In some embodiments, the therapeutic radiation dose can be delivered in fractions. Fractionation refers to spreading out the total dose of radiation over time, for example, over days, weeks or months. The dose delivered in each fraction can be about 1-15 grays per day. The treatment plan can include a fraction treatment one or more times per day, every other day, weekly, etc. depending on the treatment needs of each patient. In some embodiments, radiation is administered prior to, concurrently, or after administration of the compound of any one of claims 1-4. In some embodiments, radiation is administered at 1 gray, 2 grays, 3 grays, 4 grays, 5 grays, 6 grays, 7 grays, 8 grays, 9 grays, 10 grays, 11 grays, 12 grays, 13 grays, 14 grays, or 15 grays. In some embodiments, radiation is administered at 4-10 grays. In some embodiments, the radiation can be administered to the entire subject, especially if the tumor is dispersed or mobile. In some embodiments, the radiation can be administered locally to a tumor for example by hypofractionated tumor directed radiotherapy, external beam radiation therapy (EBRT), stereotactic body radiation therapy (SBRT), 3d conformal radiation therapy, intensity modulated radiation therapy, image-guided radiation therapy, or proton therapy.

In some embodiments, the therapy comprises one or more selected from the group consisting of alkylating agents, antimetabolites, anthracyclines, antitumor antibiotics, monoclonal antibodies, platinum agents, topoisomerase I inhibitors, topoisomerase II inhibitors, vinca alkaloids, taxanes, nucleoside analogs, angiogenesis inhibitors, antineoplastic agents, and chemotherapeutic agents.

Examples of alkylating agents include, but are not limited to, chlorambucil, cyclophosphamide, lomustine, melphalan, procarbazine, thiotepa, thiotepa, dacarbazine, procarbazine, carmustine, and busulfan.

Examples of antimetabolites include, but are not limited to, 6-mercaptopurine, 5-fluorouracil, cytarabine, methotrexate, hydroxyurea, fluoridine, 6-thioguanine, fludarabine, pentostatin, and chlorodeoxyadenosine.

Examples of anthracyclines include, but are not limited to, daunorubicin, doxorubicin, idarubicin, epirubicin, and mitoxantrone. Examples of antitumor antibiotics include, but are not limited to, bleomycin. Examples of monoclonal antibodies include, but are not limited to, alemtuzumab, bevacizumab, cetuximab, gemtuzumab, ibritumomab, panitumumab, rituximab, tositumomab, and trastuzumab.

Examples of platinum agents include, but are not limited to, cisplatin, oxaliplatin, and carboplatin.

Examples of topoisomerase I and topoisomerase II inhibitors include, but are not limited to, camptothecin, irinotecan, topotecan, amacrine, etoposide, etoposide phosphate, and teniposide.

Examples of vinca alkaloids include, but are not limited to, vincristine, vinblastine, vinorelbine, and vindesine.

Examples of taxanes include, but are not limited to, paclitaxel and docetaxel.

Examples of angiogenesis inhibitors include, but are not limited to, beracizumab, 2-methoxyestradiol, AG3340, angiostatin, antithrombin-III, anti-VEGF antibodies, batimastat, BMS-275291, CA1, canstatin, combretastatin, combretastatin-A4 phosphate, CC5013, captopril, celecoxib, dalteparin, EMD121974, endostatin, erlotininb, gefitinib, genistein, halofuginone, ID1, IDS3, IM862, imatinib mesylate, inducible protein-10, interferon-α, interleukin-12, lavendustin A, LY317615, AE-941, marimasat, mapsin, medroxyprogesterone acetate, METH-1, METH-2, neovastat, osteopontin cleavage product, PED, pigment epithelium growth factor, platelet growth factor 4, prolactin fragment, proliferin-related protein, PTK787/ZK222584, recombinant human platelet factor 4, restin, squalamine, SU5416, SU6668, suramin, taxol, tecogalan, thalidomide, tetrathiomolybdate, thrombospondin, TNP-470, troponin 1, vasostatin, VEDG1, VEGF-TPvAP, and ZD6474.

Chemotherapeutic agents that can be combined with the compounds disclosed herein include, but are not limited to, DNA damaging agents and these include topoisomerase inhibitors (e.g., etoposide, camptothecin, topotecan, irinotecan, teniposide, mitoxantrone), anti-microtubule agents (e.g., vincristine, vinblastine), antimetabolite agents (e.g., cytarabine, methotrexate, hydroxyurea, 5-fluorouracil, flouridine, 6-thioguanine, 6-mercaptompurine, fludarabine, pentostatin, chlorodeoxyadenosine), DNA alkylating agents (e.g., cisplatin, mecholorethamine, cyclophosphamide, ifosphamide, melphalan, chlorambucil, busulfan, thiotepa, carmustine, lomustine, carboplatin, dacarbazine, procarbazine) and DNA strand break inducing agents(e.g., bleomycin, doxorubicin, daunorubicin, idarubicin, mitomycin C).

Examples of chemotherapeutic agents include, but are not limited to, avicine, aclarubicin, acodazole, acronine, adozelesin, adriamycin, aldesleukin, alitretinoin, auopurinol sodium, altretamine, ambomycin, ametantrone acetate, aminoglutethimide, amsacrine, anastrazole, annonaceous acetogenins, anthramycin, asimicin, asparaginase, asperlin, azacitidine, azetepa, azotomycin, batimastat, benzodepa, bexarotene, bicalutamide, bisantrene, bisnafide, bizelesin, bleomycin, brequinar, bropirimine, bullatacin, busulfan, cabergoline, cactinomycin, calusterone, caracemide, carbetimer, carboplatin, carmustine, carubicin, carzelesin, cedefingol, chlorambucil, celecoxib, cirolemycin, cisplatin, cladribine, crisnatol, cyclophosphamide, cytarabine, dacarbazine, DACA, dactinomycin, daunorubicin, daunomycin, decitabine, denileukin, dexormaplatin, dezaguanine, diaziquone, docetaxel, doxorubicin, droloxifene, dromostalone, duazomycin, edatrexate, eflornithine, elsamitrucin, estramustine, etanidazole, etoposide, etoprine, fadrozole, fazarabine, fenretinide, floxuridine, fludarabine, fluorouracil, flurocitabine, 5-FdUMP, fosquidone, fosteuecine, FK-317, FK-973, FR-66979, FR-900482, gemcitabine, gemtuzumab, gold Au¹⁹⁸, goserelin, guanacone, hydroxyurea, idarubicin, ilmofosine, interferon-α and analogs, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, metoprine, maturedepa, mitindomide, mitocarcin, mitogillin, mitomalacin, mitomycin, mitomycin C, mitosper, mitotane, mitoxantrone, mycophenolic acid, nocodazole, nogalamycin, oprelvekin, ormaplatin, oxisuran, ozogamacin, paclitaxel, pamidronate, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rituximab, rogletimide, rolliniastatin, safingol, samarium, semustine, simtrazene, sparfosate, sparsomycin, spirogermanium, spiromustine, spiroplatin, squamocin, squamotacin, streptonigrin, streptozocin, SrC12, sulphofenur, talisomycin, taxane, toxoid, tecoglan, tegafur, teloxantrone, temoporfin, teniposide, teroxirone, testolactone, thiamiprine, thiotepa, thymitaq, tiazofurin, tirapazamine, tomudex, Top-53, topotecan, toremixifme, trastuzumab, trestolone, triciribine, triciribine, trimetrexate, trimetrexate glucuronate, triptorelin, tubulozole, uracil mustard, uredepa, valrubicin, vapreotide, vinblastine, vincristine, vindesine, vinepidine, vinglycinate, vinleurosine, vinorelbine, vinrosidine, vinzolidine, vorozole, zeniplatin, zinostatin, zorubicin, 2-cholrodeoxyrubicine, 2′-deoxyformycin, 9-aminocamptothecin, raltitrexed, N-propargyl-5,8-didezafolic acid, 2-cholo-2′arabinofluoro-2′ deoxyadenosine, 2-cholo-2′-deoxyadenosine, anisomycin, trichostatin, hPRL-G129R, CEP-751, linomide, sulfur mustard, nitrogen mustard, N-methyl-N-nitrosourea, fotemustine, streptozotocin, mitozolomide, temozolomide, AZQ, CI-973, DWA21 14R, JM216, JM335, bisplatinum, cytrabincine, 6-mercaptopurine, hypoxanthine, CPT-11, epirubicin, darubicin, pyrazoloacridine, all-trans-retinol, 14-hydroxy-retro-retinol, all-trans retinoic acid, N-(4-hydroxyphenyl) retinamide, 13-cis-retinoic acid, 3-methyl TTNEB, 9-cis-retenoic acid, 2-Cda, 20-epil,25-dihydroxyvitamin-D3, 5-ethynyl uracil, abiraterone, acylfulvene, adecylpenol, ALL-TK antagonists, ambumastine, amidox, amifostine, amino levulinic acid, anagrelide, andrographolide, antagonists D, antarelix, anti-dorsalizing morphogenetic protein-1, antiandrogen, antiestrogen, antineoplastone, antisense oligonucleotides, aphidicolin, apoptosis gene modulators, apoptosis regulators, apurinic acid, ara-cdp-dl-PTBA, arginine aminase, asulacrine, atamestine, atrimustine, axinamastine 1, axinamastine 2, axinamastine 3, azasetron, azatoxin, azatyrosine, baccatin III derivatives, balanol, BCR/ABL antagonist, benzochlorins, benzoylsaurosporine, beta lactam derivatives, beta-alethine, pentomone, perillyl alcohol, phenozenomyein, phenyl acetate, phosphatase inhibitors, picibanil, pilocarbine and salts and analogs thereof, pirarubucin, piritrexim, piritrexim isothiocyanate, placetin A, placetin B, plasminogen activator inhibitor, platinum complex, phenyl ethyl isothiocyanate and analogs thereof, platinum triamine complex, podophylotoxin, porfimer sodium, propyl bis acridones, prostaglandin J2, protease inhibitors, protein A based immune modulators, PKC inhibitors, microalgal, protein tyrosine phosphatase inhibitors, purine nucleoside phosphorylase inhibitors, purpurins, pyridoxylated hemoglobin polyoxyethylene conjugate, raf antagonists, raltitrexed, ramosetron, ras farnesyl protein transferase inhibitors, ras inhibitors, ras-GAP inhibitors, ratellitptine demethylated, RBX2258, Rhenium Re¹⁸⁶ etidronate, rhizoxine, ribozymes, RII retinide, rosagliatazone and analogs and derivatives thereof, rohitukine, romurtide, roquinimex, rubiginone Bl, ruboxyl, saintopin, SarCNU, sarcophytol A, sargrmostim, sdi 1 mimetics, senescence derived inhibitor 1, sense oligonucleotides, signal transduction inhibitors, signal transduction modulators, single chain antigen binding protein, sitogluside, sizofiran, sobuzoxane, sodium borocaptate, sodium phenyl acetate, solverol, somatomedin binding protein, sonermin, spicamycin D, splenopentine, spongistatin 1, squalamine, stem cell inhibitors, stem cell division inhibitors, stipiamide, stromelysin, sulfinosine, superactive vasoactive intestinal peptide antagonists, suradista, siramin, swainsonine, synthetic glycosaminoglycans, tallimustine, tamoxifen methiodide, tauromustine, tazarotene, tacogalan sodium, tellurapyrilium, telomerase inhibitors, tetrachlorodecaoxide, tetrazomine, thaliblastine, thalidomide, thiocoraline, thrombopoetin and mimetics thereof, thymalfasin, thymopoetin receptor agonist, thymotrinan, thyroid stimulating harmone, tin ethyl etiopurpin, titanocene and salts thereof, tomsulosine, topsentin, toremifene, totipotent stem cell factors, translation inhibitors, tretinoin, triacetyluridine, tropisetron, turosteride, tyrosine kinase inhibitors, tyrphostins, UBC inhibitors, ubenimex, urogenital sinus derived growth inhibitory factor, urokinase receptor antagonists, variolin B, vector system, erythrocyte gene therapy, velaresol, veramine, verdins, verteporfin, vinorelbine, vinxaltine, vitaxin, zanoterone, zilascorb zinostatin, ¹²⁵I fibrinogen, ¹⁸F fludeoxyglucose, ¹⁸F fluorodopa, ¹²⁵I insulin, ¹²³I iobenguane, ¹³¹I iodipamine sodium, ¹³¹I iodoantipyrine, ¹³¹I iodocholesterol, ¹²⁵I iodopyracet, ¹²³I iofetamine HCl, ¹³¹I iomethin, ¹²⁵I iothalamate sodium, ¹³¹I iothalamate, ¹³¹I iotyrosine, ¹²⁵I liothyronine, ¹⁹⁷Hg merosproprol, ¹³¹I methyl iodobenzoguanine, ⁷⁵Se selenomethionine, ^{99m}Tc technetium furifosmin, ^{99m}Tc technetium gluceptate, ^{99m}Tc technetium biscisate, ^{99m}Tc technetium disofenin, ^{99m}Tc technetium lidofenin, ^{99m}Tc technetium mebrofenin, ^{99m}Tc technetium medronate and sodium salts thereof, ^{99m}Tc technetium sestambi, ^{99m}Tc technetium siboroxime, ^{99m}Tc technetium succimer, ^{99m}Tc technetium sulfur colloid, ^{99m}Tc technetium teboroxime, ^{99m}Tc technetium tetrofosmin, ^{99m}Tc technetium tiatide, ¹²⁵I thyroxine, ¹³¹I thyroxine, ¹³¹I tolpovidone, ¹²⁵I triolein, and ¹³¹I triolein.

In some embodiments, the therapy comprises one or more immune checkpoint inhibitors. Immune checkpoint inhibitors include any agent that blocks or inhibits, in a statistically significant manner, the inhibitory pathways of the immune system. Immune checkpoint inhibitors include antibodies or antigen binding fragments thereof, other binding proteins, biologic therapeutics, and small molecules, that bind to and block or inhibit the activity of a target. Examples of immune checkpoint targets for blocking or inhibition include, but are not limited to, CTLA-4, PD-L1, PD-L2, PD-1, BTLA, HVEM, GAL9, LAG3, TIM3, VISTA, KIR, 2B4, CD160 (also referred to as BY55), CGEN-15049, CHK1 kinase, CHK2 kinase, A2aR, and various B-7 family ligands. Examples of B-7 family ligands include, but are not limited to, B7-1, B7-2, B7-DC, B7H1, B7-H2, B7-H3, B7-H4, B7-H5, B7-H6, and B7-H7. Examples of immune checkpoint inhibitors include, but are not limited to, tremelimumab, anti-OX40, anti-B7-H1, MEDI4736, MK-3475, nivolumab, CT-011, BY55, AMP224, BMS-936559, MPLDL3280A, MSB0010718C, ipilimumab, pembrolizumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and BMS-986189.

In some embodiments, the present invention provides methods of inhibiting angiogenesis in a cancer. In some embodiments, the method comprises administering to a subject a compound of the present invention. In some embodiments, the method comprises administering to a subject a compound of the present invention, wherein the subject has cancer.

In some embodiments, the present invention provides methods of sensitizing a cancer or tumor. In some embodiments, the cancer or tumor is sensitized to radiation therapy. In some embodiments, the cancer or tumor is resistant to radiation therapy. In some embodiments, the cancer or tumor is sensitized to one or more chemotherapeutic agents. In some embodiments, the method comprises administering to a subject a compound of the present invention. In some embodiments, the method comprises administering to a subject a compound of the present invention, wherein the subject has a cancer or tumor. In some embodiments, the cancer or tumor is resistant to radiation or one or more chemotherapeutic agents.

### Pharmaceutical Compositions

In some aspects, the present invention provides compositions useful for reducing LCMT-1. In some embodiments, the composition comprises one or more inhibitors of LCMT-1. In some embodiments, the one or more inhibitors of LCMT-1 reduce the expression or activity of one or more proteins involved in LCMT-1. These proteins can include, but are not limited to, protein phosphatase-2A (PP2A), B55α (PR55α) regulatory subunit, and PP2A methyl esterase (PME-1). In some embodiments, the one or more inhibitors of LCMT-1 reduce the interaction of LCMT-1 with S-adenosylmethionine (SAM).

In some embodiments, the one or more inhibitors of LCMT-1 are one or more inhibitors of protein phosphatase-2A (PP2A) methylation. In some embodiments, the one or more inhibitors disrupt the interaction of PP2A and B55α (PR55α) regulatory subunit.

In some embodiments, the composition of the present invention comprises a pharmaceutical composition. In some embodiments, the inhibitor of LCMT-1 to be delivered to the subject comprises a pharmaceutical composition. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents include growth factors, hormones, anti-inflammatories including corticosteroids, immunosuppressants, and the like.

Controlled- or sustained-release formulations of a pharmaceutical composition of the invention may be made using conventional technology.

For oral application, particularly suitable are tablets, dragees, liquids, drops, or capsules, caplets and gelcaps. Other formulations suitable for oral administration include, but are not limited to, a powdered or granular formulation, an aqueous or oily suspension, an aqueous or oily solution, a paste, a gel, a toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents, fillers, lubricants, disintegrates, or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose, or hydrogenated edible fats); emulsifying agent (e.g., lecithin or acacia); non-aqueous vehicles (e.g., almond oil, oily esters, or ethyl alcohol); and preservatives (e.g., methyl or propyl p-hydroxy benzoates or sorbic acid). Liquid formulations of a pharmaceutical composition of the invention that are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists of the use of materials that are solid or semi-solid at room temperature (i.e., having a relatively low softening or melting point range) to promote granulation of powdered or other materials, essentially in the absence of added water or other liquid solvents. The low-melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

Tablets may comprise multi-layer tablets comprising a layer providing for the delayed release of one or more compounds of the invention, and a further layer providing for the immediate release of a medication for treatment of a disease. Using a wax/pH-sensitive polymer mix, a gastric insoluble composition may be obtained in which the active ingredient is entrapped, ensuring its delayed release.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally administrable formulations that are useful include those that comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. By way of example, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Exemplary Embodiments

This invention provides the following non-limiting embodiments.

Embodiment 1 is a compound of Formula (I), or a derivative, prodrug, or pharmaceutically acceptable salt or solvate thereof:

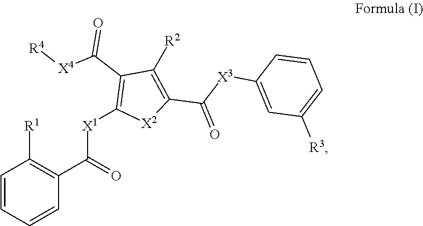

wherein:
R¹, R², R³, and R⁴ are each independently selected from the group consisting of CH₃, CD₃, and CF₃;
X¹, X², X³, and X⁴ are each independently selected from the group consisting of O, NR^{X}, and CR^{X}₂; and
each instance of R^{X} is independently selected from the group consisting of H, D, F, CH₃, CD₃, and CF₃.

Embodiment 2 is the compound of embodiment 1, wherein X¹ is NH.

Embodiment 3 is the compound of embodiment 1 or embodiment 2, wherein X² is S.

Embodiment 4 is the compound of any one of embodiments 1 through 3, wherein X³ is NH.

Embodiment 5 is the compound of any one of embodiments 1 through 4, wherein X⁴ is O.

Embodiment 6 is the compound of any one of embodiments 1 through 5, wherein at least one of R¹, R², R³, and R⁴ is CH₃.

Embodiment 7 is the compound of any one of embodiments 1 through 6, wherein R¹, R², R³, and R⁴ are all CH₃.

Embodiment 8 is the compound of any one of embodiments 1 through 7, wherein the compound Formula (I) is a compound of Formula (II):

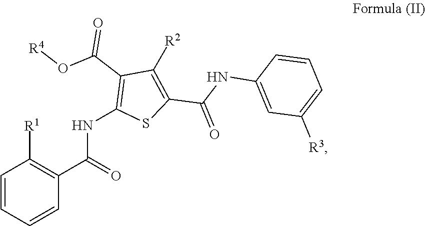

wherein R¹, R², R³, and R⁴ are each independently selected from the group consisting of CH₃, CD₃, and CF₃.

Embodiment 9 is the compound of any one of embodiments 1 through 8, wherein at least one of R¹, R², R³, and R⁴ is CH₃.

Embodiment 10 is the compound of any one of embodiments 1 through 9, wherein R¹, R², R³, and R⁴ are all CH₃.

Embodiment 11 is a method of decreasing leucine carboxyl methyltransferase-1 (LCMT-1) activity in a subject comprising administering to the subject the compound of any one of embodiments 1-10.

Embodiment 12 is a method of treating a disease or disorder in a subject comprising administering to the subject the compound of any one of embodiments 1-10.

Embodiment 13 is the method of embodiment 12, wherein the disease or disorder is cancer.

Embodiment 14 is the method of embodiments 12 or 13, wherein the cancer is selected from the group consisting of squamous cell carcinoma, melanoma, lung, leukemia, colorectal, breast, or pancreatic cancer.

Embodiment 15 is the method of any one of embodiments of 12 through 14, wherein the compound of Formula (I) is administered in combination with one or more therapies selected from the group consisting of radiation therapy, surgery, chemotherapy, and immune checkpoint inhibitors.

Embodiment 16 is the method of any one of embodiments 12 through 15, wherein the therapy is radiation therapy.

Embodiment 17 is the method of any one of embodiments 12 through 16, wherein the method comprises: a) administering to said subject the compound of any one of claims 1-10; and b) administering to said subject an effective amount of radiation therapy.

Embodiment 18 is the method of any one of embodiments 12 through 17, wherein the radiation therapy is administered by way of a regimen selected from the group consisting of: 4-10 grays.

Embodiment 19 is a method of sensitizing a tumor in a subject to treatment comprising administering to the subject the compound of any one of embodiments 1-10.

### Experimental Examples

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples, therefore, point out specific embodiments of the present invention, and are not to be construed as limiting in any way the remainder of the disclosure.

### Example 1: a Small Molecule Inhibitor of Leucine Carboxyl Methyltransferase-1 Inhibits Cancer Cell Survival

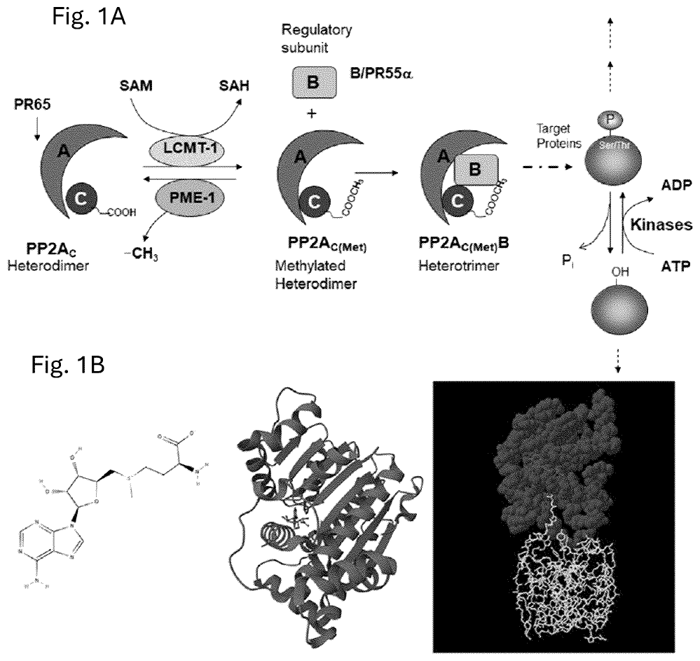

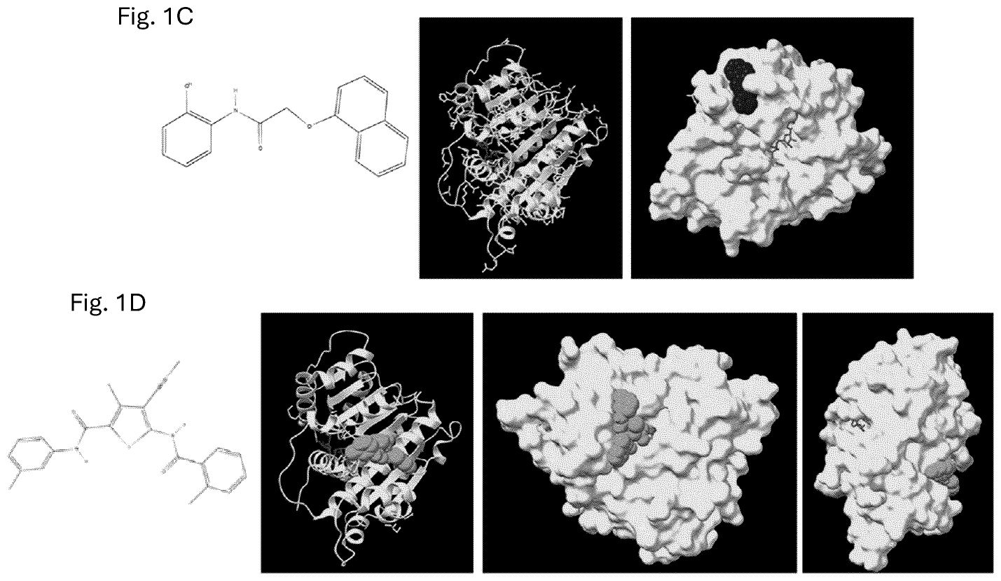

Methylation of PP2Ac is controlled by two enzymes, a methyl transferase (leucine carboxyl methyltransferase-1 (LCMT-1)), which adds the methyl group with S-adenosylmethionine (SAM) as the methyl donor; and an esterase (PP2A methyl esterase (PME-1)), which removes the methyl group (FIG. 1A-FIG. 1C) (Lee et al. The Journal of biological chemistry. 2007; 282(42):30974-84; Sangodkar et al. The FEBS journal. 2016; 283(6):1004-24; Stanevich et al. Mol Cell. 2011; 41(3):331-42; Lee et al. Science signaling. 2018; 11(512)). Methylation and demethylation of PP2Ac modulates PP2A activity. LCMT-1 is necessary for normal progression through mitosis and its overexpression has been associated with poor prognosis in hepatocellular carcinoma (Lee et al. The Journal of biological chemistry. 2007; 282(42):30974-84; Zhang et al. Translational oncology. 2023; 27:101572). The ability of a small molecule inhibitor of LCMT-1 to inhibit cancer cell survival in a manner similar to LB-100 was tested. While LB-100 inhibits the catalytic subunit of PP2A, the LCMT-1 inhibitor can indirectly inhibit PP2A heterotrimers containing the B55α regulatory subunit by preventing PP2Ac methylation.

The materials and methods are now described.

### Library Screening

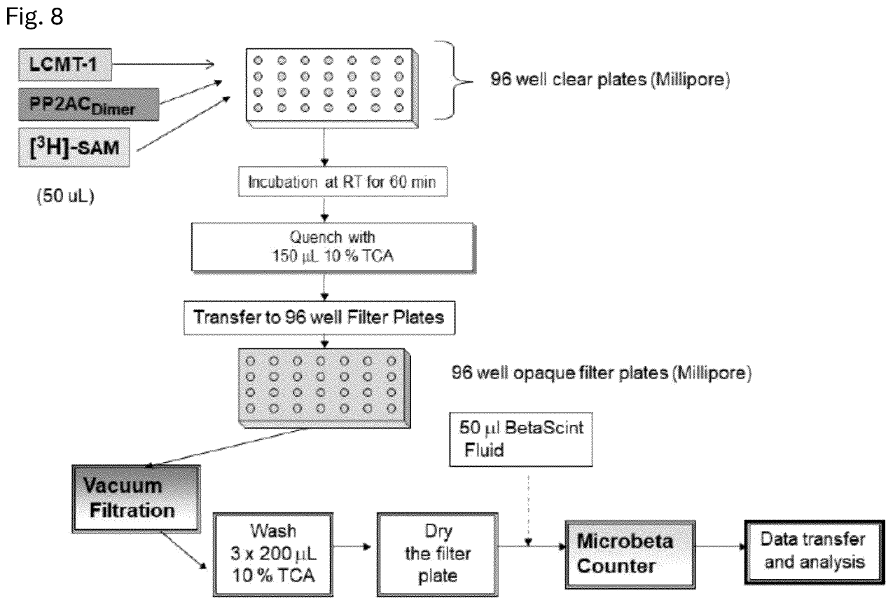

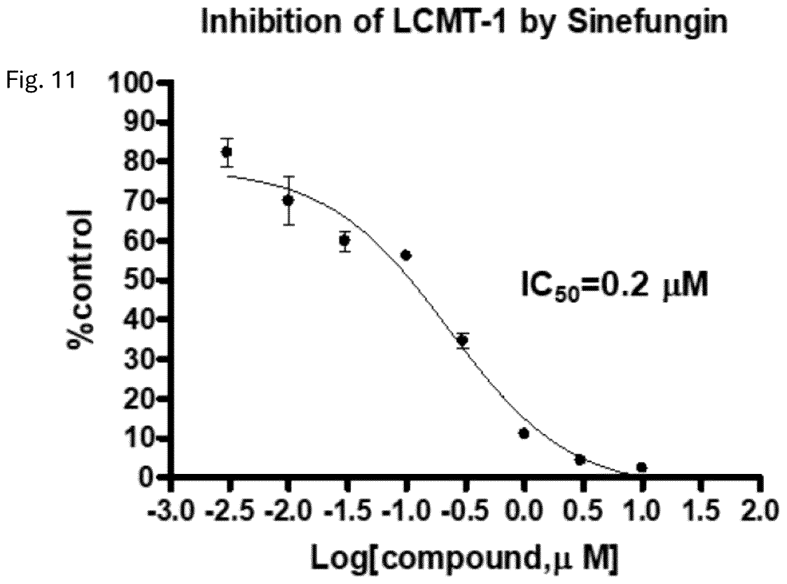

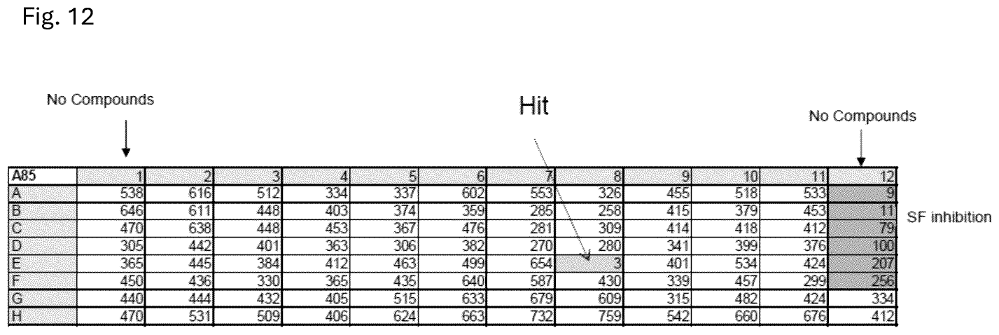

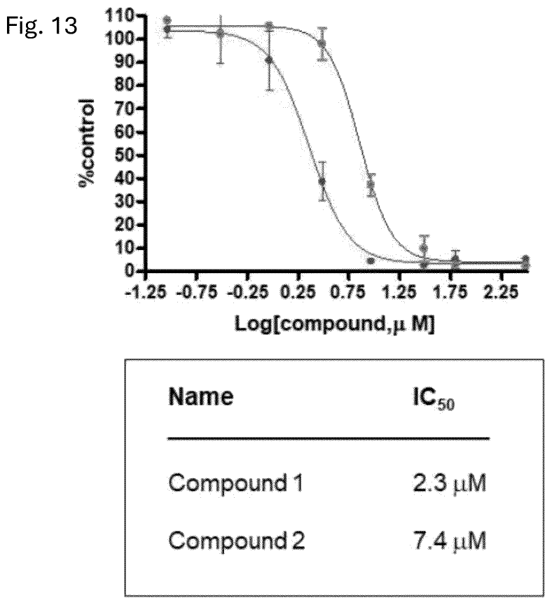

Small molecule libraries (ASINEX and CHEM DIV03) comprising a total of 36,550 compounds that were not SAM analogues were screened using a radioactive LCMT-1 activity assay (FIG. 8-10). This screen identified two potential LCMT-1 inhibitors, N-(2-Hydroxyphenyl)-2-(1-naphthal enyloxy)acetamide (henceforth referred to as Compound 1) and (3-Thiophenecarboxylic acid, 4-methyl-2-[(2-methylbenzoyl)amino]-5-[[(3-methylphenyl)amino]carbonyl]-, methyl ester (also known as Methyl 4-methyl-2-[(2-methylbenzoyl)amino]-5-[[(3-methylphenyl)amino]carbonyl]-3-thiophenecarboxylate), henceforth referred to as Compound 2, FIG. 1D and E, FIG. 11-13).

### Molecular Modeling

The x-ray crystallographic structures of human LCMT-1, the SET (catalytic) domain of human histone-lysine-N-methyltransferase 2E (MLL5 methyltransferase), and both chains of the heterodimeric acidic residue methyltransferase 1 (ARMT1) were downloaded from the RCSB Protein Data Bank (Berman et al. Nucleic acids research. 2000; 28(1):235-42). The SET domain is conserved in class II and III methyltransferases (Katz et al. MCP. 2003; 2(8):525-40). Like LCMT-1, both chains of ARMT1 contain a Rossman fold catalytic domain characteristic of class I methyltransferases (Katz et al. MCP. 2003; 2(8):525-40; Dennis et al. J Struct Biol. 2020; 212(1):107576). Protein-ligand interactions between the SET domain of MLL5 and Compound 2, the SET domain of MLL5 and SAM, both chains of ARMT1 and Compound 2, and both chains of ARMT1 and SAM were modelled with SwissDock software (Grosdidier et al. Nucleic acids research. 2011; 39:W270-7).

Because interactions between LCMT-1, and Compounds 1 and 2 could not be analyzed with SwissDock due to the size of LCMT-1, these interactions were analyzed with AutoDock Vina software (Eberhardt et al. J Chem Inf Model. 2021; 61(8):3891-8, Trott et al. J Comput Chem. 2010; 31(2):455-61). Outputs from SwissDock and AutoDock Vina were converted to Protein Data Bank file format with OpenBabel software (O'Boyle et al. J Cheminform. 2011; 3:33). Outputs were visualized with University of California San Francisco (UCSF) ChimeraX software (Meng et al. Protein Sci. 2023; 32(11):e4792; Pettersen et al. Protein Sci. 2021; 30(1):70-82; Goddard et al. Protein Sci. 2018; 27(1):14-25).

### Lcmt-1 Activity Assays

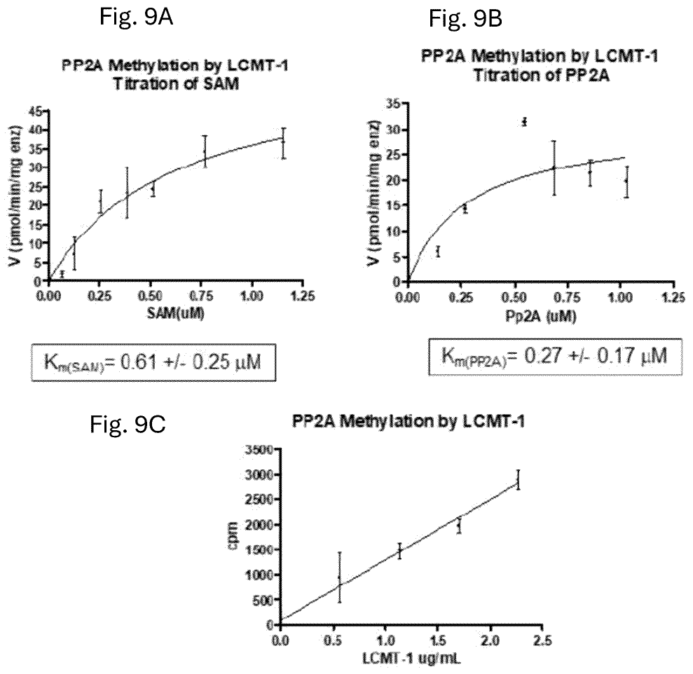

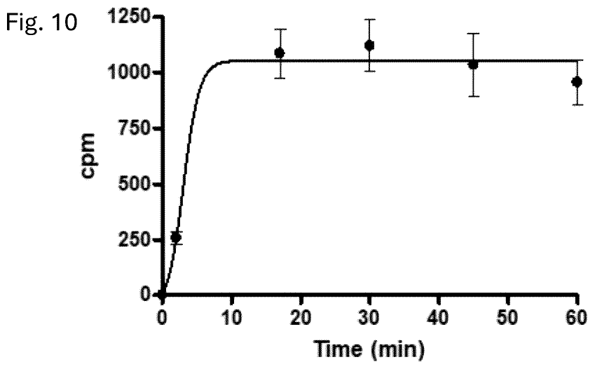

Recombinant human LCMT-1 (rhLCMT-1) and rhPP2Ac were obtained from SignalChem (Richmond, British Columbia, Canada). Methyltransferase assays were performed with 100 ng rhLCMT-1 and 91.62 ng rhPP2Ac (2:1 molar ratio) (28) in reaction buffer containing 20 mM Tris (pH 8.0), 50 mM NaCl, 1 mM EDTA. rhLCMT-1 was incubated with Compound 1 (Chemspace, San Jose, California), Compound 2 (Akos Consulting and Solutions, Amtsgericht, Freiburg, Germany) or sinefungin, a SAM analogue, (Active Motif, Carlsbad, California), each at 50 μM final concentration, for 10 minutes at room temperature. rhPP2Ac was added to the reactions with 20 μM (final concentration) SAM (New England Biolabs, Ipswich, Massachusetts, FIG. 9). The negative control reaction did not contain SAM. Reactions were incubated at 37° C. for 30 minutes (FIG. 10). Because the inhibitors gave false positive results with the secondary reactions used in colorimetric and bioluminescent methyltansferase assays, LCMT-1 activity was assessed using dot immunoblots. Reactions were applied to nitrocellulose membranes (Cytiva, Marlborough, Massachusetts). The membranes were blocked with tris-buffered saline (TBS) with 5% nonfat dry milk. Membranes were probed with an antibody directed to demethylated PP2Ac (Santa Cruz Biotechnology, Dallas, Texas, catalog #sc-13601, 1:1,000 dilution). Membranes were treated with infrared (IR)-dye anti-mouse antibody (Li-Cor, Lincoln, Nebraska, catalog #926-32210, 1:20,000 dilution) and densities were measured with Li-Cor Image Studio (version 5.2) software. rhLCMT-1 inhibition assays were performed three times, each time with five replicates for each condition.

### Cell Culture

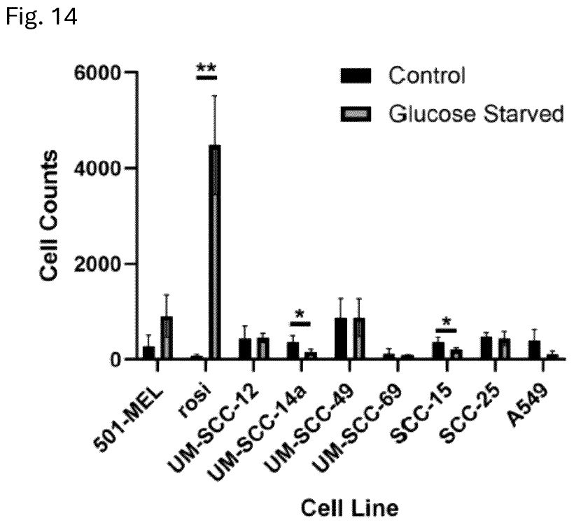

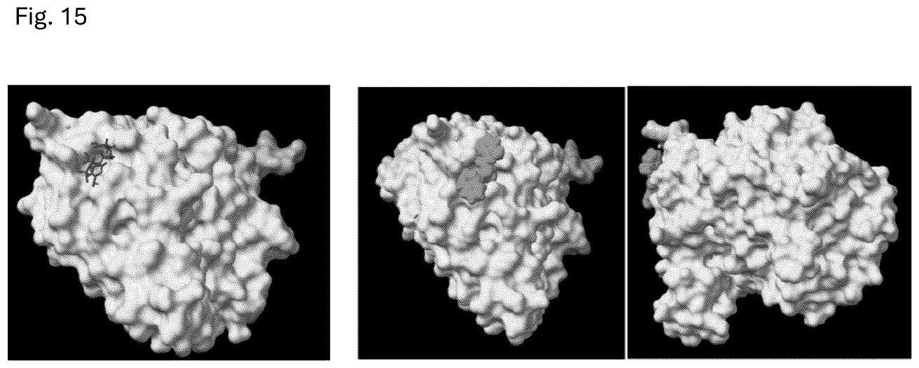

The human MDA-MB-231 and MCF7 breast carcinoma cell lines were obtained from the Fox Chase Cancer Center cell culture core facility. These cell lines have been previously shown to be sensitive to glucose starvation-mediated PR55a depletion (Di Conza et al. Cell Rep. 2017; 18(12):2836-44). Additional cell lines (501-MEL, Rosi, UM-SCC-12, UM-SCC-14a, UM-SCC-49, UM-SCC-69, SCC-15, SCC-25, A549) were screened for sensitivity to PR55a depletion with glucose starvation assays. Rosi melanoma cells were susceptible to glucose starvation (FIG. 14). SCC-15 and SCC-25 cells were grown in Dulbecco's modified Eagle's medium (DMEM) with Ham's F-12 (DMEM/F-12, Corning®, Glendale, Arizona, catalog #90-090-PB) with 10% fetal bovine serum (FBS, Corning® catalog #35-015-CV). The remaining cells were grown in DMEM (Corning® catalog #50-003-PC) with 10% FBS. Cells were maintained in a 5% carbon dioxide atmosphere at 37° C. and tested free of mycoplasma contamination. Cells were not maintained in culture for more than 6 months without cryopreservation.

### Western Blots

MCF7, MDA-MB-231 and Rosi cells were treated with Compound 1 or Compound 2 at a concentration of 20 μM in complete medium, and lysates were harvested after 2, 4, 6, 8 and 12 hours in radioimmunoprecipitation assay buffer containing protease inhibitors (Boster Bio, Pleasanton, California, catalog #AR1182-1) and phosphatase inhibitors (Santa Cruz Biotechnology catalog #sc-45044 and #sc-45045). Protein concentrations were measured with a bicinchoninic acid assay (G-Biosciences, Saint Louis, Missouri). Fifty micrograms of each lysate were used for bis-tris polyacrylamide gel electrophoresis. Resolved lysates were transferred to nitrocellulose membranes. Membranes were blocked with TBS with 5% bovine serum albumin before incubation with primary antibodies.

Antibodies obtained from Cell Signaling Technology (Danvers Massachusetts) were, anti-PP2Ac (catalog #2038, 1:1,000 dilution), anti-phospho-adenosine monophosphate-activated protein kinase (pAMPKα2 threonine 172, catalog #2535, 1:1,000 dilution). Antibodies obtained from Proteintech (Rosemont, Illinois) include anti-phospho-receptor-interacting serine/threonine protein kinase-1 antibodies (pRIPK1 serine 161 (catalog #66854-1-Ig, 1:1,000 dilution) and pRIPK1 serine 166 (catalog #28252-1-AP, 1:10,000 dilution), anti-mixed lineage kinase domain like pseudokinase (MLKL, catalog #66675-1-Ig, 1:5,000 dilution), anti-AMPKα2, anti-phospho-acetyl-coenzyme A carboxylase (pACC1 serine 79, catalog #29119-1-AP, 1:1,000 dilution), anti-ACC1 (catalog #21923-1-AP, 1:2,000 dilution) and anti-β-actin (catalog #66009-1-Ig, 1:50,000 dilution). The anti-RIPKI antibody was obtained from AbClonal (Woburn, Massachusetts, catalog #A7414, 1:750 dilution). The anti-phospho-MLKL antibody was obtained from R&D Systems (Minneapolis, Minnesota, 1:1,000 dilution). The LCMT-1 antibody was obtained from Origene (Rockville, Maryland, catalog #TA503127S, 1:2,000 dilution). Appropriate IR-dye secondary antibodies were used (Alexa Fluor® 790-conjugated anti-mouse (catalog #115-655-146, 1:50,000 dilution), Alexa Fluor® 680-conjugated anti-mouse (catalog #115-625-146, 1:50,000 dilution), and Alexa Fluor® 680-conjugated anti-rabbit (catalog #115-625-144, 1:50,000 dilution) were obtained from Jackson ImmunoResearch Laboratories (West Grove, Pennsylvania). Band densities were normalized to β-actin, the endogenous control. At least five replicates were used for statistical analyses.

### Cytotoxicity Assays

HS-5, HEK-293, MCF7, MDA-MB-231 and Rosi cells were seeded in 96-well optical bottom tissue culture plates (Corning, New York) at a density of 2.5×10³ cells/well. After 24 hours, the media were changed to media containing LB-100 (Cayman Chemical Company, Ann Arbor, Michigan) or Compound 2 at various concentrations (50-0.31 μM). Control cells were given fresh media. After 48 hours the media were aspirated and plates were washed with warmed Dulbecco's phosphate-buffered saline (DPBS) with calcium and magnesium (Sigma-Aldrich, catalog #D1283). Cells were stained with 3 μM calcein-AM (AAT Bioquest, Pleasanton, California, catalog #22002) in DPBS for 30 minutes in the dark at 37° C. Plates were immediately read with a Biotek fluorescent plate reader (excitation 485 nm, emission 520 nm). The cytotoxicity assays were performed at least three times, each time with six replicates per condition.

### Transfections

A mammalian LCMT-1 plasmid expression vector was obtained from Origene (pCMV6-LCMT1, catalog #RC200018). HEK-293 cells were transfected with the plasmid using PolyJet™ transfection reagent (SignaGen Laboratories, Frederick, Maryland) according to manufacturer's directions. After 5 hours, the medium was changed. The next day, the transfected cells were seeded in 96-well optical bottom tissue culture plates for cytotoxicity assays as previously described. After 24 hours, the medium was changed to medium containing Compound 2 at various concentrations (0.78-50 μM), or fresh medium with the carrier (dimethyl sulfoxide) in the case of the control. After 48 hours, cells were stained with calcein-AM and fluorescence was measured.

MDA-MB-231 cells were also transfected with the LCMT-1 expression vector using Lipofectamine 3000 transfection reagent (Thermo Fisher Scientific, Waltham, Massachusetts) according to the manufacturer's transfection protocol for MDA-MB-231 cells. After 5 hours the medium was changed. The cells were split 1:100 the following day, and 2 days after transfection, selection with G418 sulfate (Mediatech, Herndon, Virginia) at 700 ng/mL for 2 weeks commenced to develop stably transfected cells. After the selection process, cells were used in Compound 2 cytotoxicity assays as previously described.

### Colony Forming Assays

MCF7, MDA-MB-231 and Rosi cells were seeded in 96-well tissue culture plates (Celltreat, Pepperell, Massachusetts) at a density of 90 cells per well (Mayr et al. Int J Mol Sci. 2018; 19(3)). After 24 hours, the media were changed to media containing LB-100 or Compound 2 at various concentrations (50-3.25 μM). Control cells were given fresh media. After one week the media were aspirated and the wells were washed with PBS. Cells were stained with crystal violet solution (0.25 g crystal violet in 20% methanol) for 10 minutes. The wells were washed 6 times with PBS to remove excess crystal violet. Colonies were counted with an ECHO Revolve hybrid microscope at 1.5× magnification. There were ten replicates for each condition.

### Cell Cycle Analyses

MCF7, MDA-MB-231 and Rosi cells were seeded in 6-well tissue culture plates (Celltreat) at a density of 5×10⁵ cells per well. After 24 hours, the DMEM was changed to media containing 20 μM LB-100 or Compound 2. Control cells were given fresh media. After 24 hours, cells were labeled with 50 μM bromodeoxyuridine (BrdU, BioGems, Westlake Village, California, catalog #5911439). Cells were lifted with 0.25% trypsin, 2.21 mM ethylenediaminetetraacetic acid (Corning catalog #25-053-CI) after 4 hours and fixed with 70% ethanol overnight at 4° C. Cells were pelleted and cellular DNA was denatured with HCl. After neutralization with 1 M Tris, pH 8.5, cells were labeled with FITC-anti-BrdU (Biolegend, San Diego, California, catalog #364104, 5 μL/100 μL cell suspension) and 7-aminoactinomycin-D (7-AAD, Biolegend, catalog #420404, 1 μL/100 μL cell suspension). Cells were analyzed with a Becton-Dickinson FACSymphony™ flow cytometer. Cell cycle analyses were performed three times.

### Annexin V Assays

MCF7, MDA-MB-231 and Rosi cells were treated with LB-100 and Compound 2 at 5-20 μM concentrations. After 48 hours, cells were lifted with Accutase and washed twice with PBS (pH 7.4) with 10% FBS. Cells were resuspended in annexin V binding buffer (10 mM HEPES (pH 7.4), 140 mM NaCl and 2.5 mM CaCl₂)) and labeled with FITC-anti-Annexin V and 7-AAD (Biolegend, FITC Annexin V Apoptosis Detection Kit with 7-AAD, catalog #640922). Cell surface annexin V expression was assessed with flow cytometry.

Statistical analyses were performed with Prism Graphpad version 10.0.1.218 software. All data were subjected to normality and lognormality tests (Shapiro-Wilk and Kolmogorov-Smirnov tests) prior to comparison of means. One-way analyses of variance (ANOVA) with Dunnett's multiple comparisons test were used for normally distributed data, while Brown-Forsythe and Welch ANOVAs with Dunnett's T3 multiple comparisons tests were used for data that were not normally distributed. A two-way ANOVA was used to compare cytotoxicity between Compound 2 and LB-100 treated cells, and between cells overexpressing LCMT-1 and controls.

The results of example experiments are now described.

Compounds 1 and 2 do not interact with LCMT-1 at the binding site of SAM nor the PP2Ac carboxyl terminus. Molecular modeling of Compound 1 and 2 interactions with LCMT-1 using AutoDock Vina yielded one binding site for each compound, with 9 different conformations at the binding site for the ligands. The conformations with the lowest binding free energies were visualized with UCSF ChimeraX software (FIGS. 1C and 1D). Molecular modeling revealed that neither Compound 1 nor Compound 2 occupied the SAM binding site of LCMT-1, or the binding site of the PP2Ac carboxyl terminus. Compound 1 appeared to occupy a cleft in the LCMT-1 protein near the SAM binding site, while Compound 2 occupied a cleft near the PP2Ac carboxyl terminus binding site.

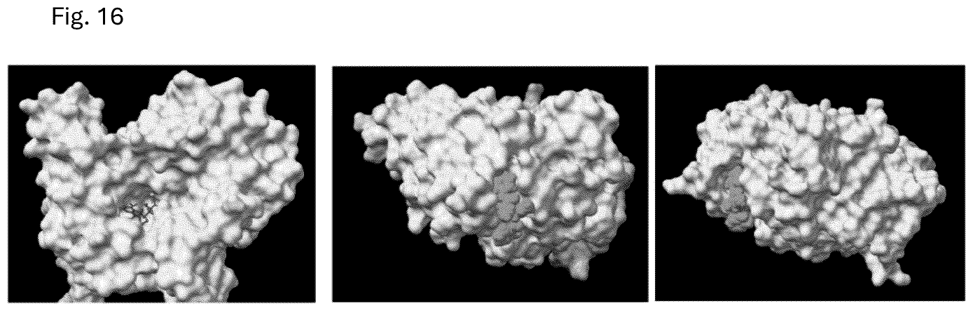

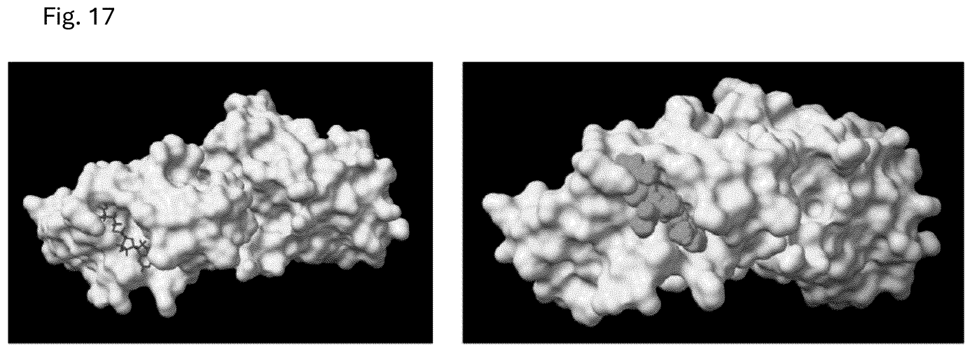

Molecular modeling of Compound 2 binding with the SET domain of MLL5 methyltransferase using SwissDock yielded 3 possible binding sites with 10 ligand conformations for each binding site. Like the modeling results for MLL5 methyltransferase, SwissDock yielded 3 possible binding sites for each chain of ARMT1 with 10 ligand conformations for each binding site. The conformation/binding site with the lowest free energy and highest SwissDock Full Fitness rank was visualized with UCSF ChimeraX software. The modelling revealed that Compound 2 may dock at the SAM binding site in the SET domain of MLL5, and partially so in the a chain of ARMT1. Compound 2 may also partially dock in the R chain of ARMT1 (FIGS. 15-17).

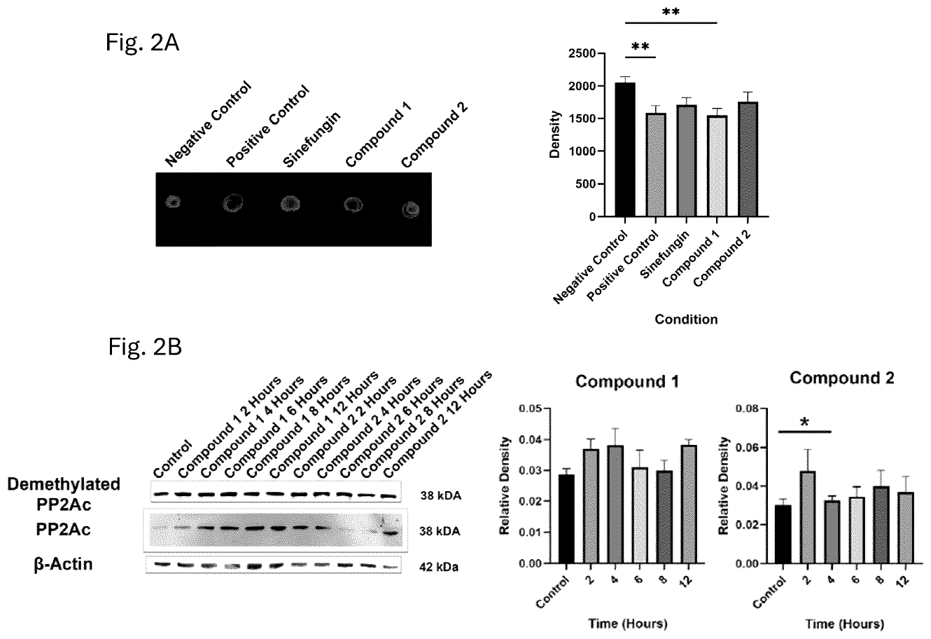

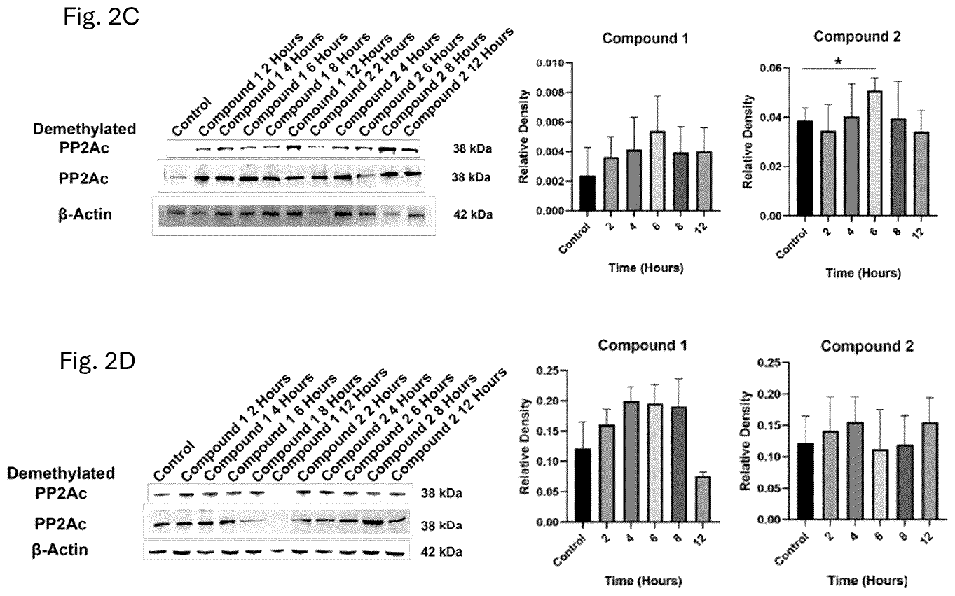

Compound 2 inhibits LCMT-1-mediated methylation of PP2Ac. rhLCMT-1 activity assays revealed that Compound 2, but not Compound 1, inhibited methylation of rhPP2Ac to the same degree as the SAM analogue sinefungin, a natural compound used as a global inhibitor of all methyltransferases (p=0.0024, FIG. 2A). rhLCMT-1 activity in Compound 2- and sinefungin-containing reactions did not differ from the negative control.

Compound 1 did not increase PP2Ac methylation in MCF7 (p=0.0716), MDA-MB-231 (p=0.0730) or Rosi cells (p=0.5517) in a statistically significant manner. Treatment with Compound 2 resulted in decreased PP2Ac methylation as compared to control in MDA-MB-231 cells which was significant at 4 hours (p=0.0431, FIG. 2B), and in Rosi cells at 6 hours (p=0.0335, FIG. 2C). Compound 2 did not significantly decrease methylation of PP2Ac in MCF7 cells (p=0.7768, FIG. 2D). Because Compound 1 did not significantly inhibit PP2Ac methylation in the LCMT-1 activity assays nor in the cell lines, Compound 2 was used for further analyses.

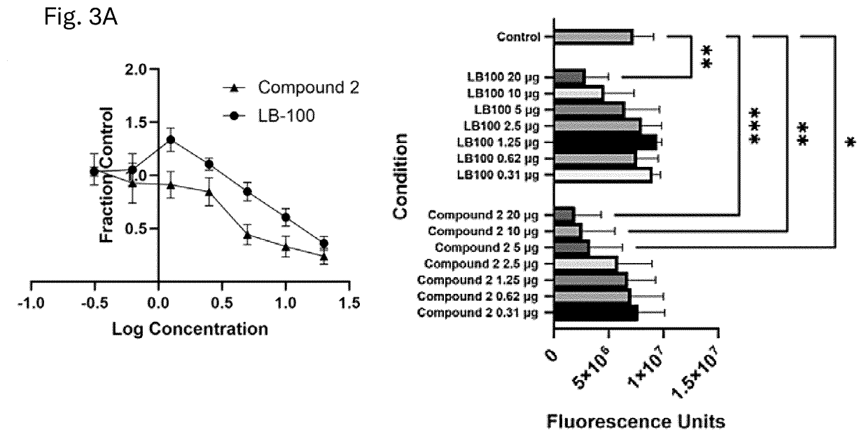

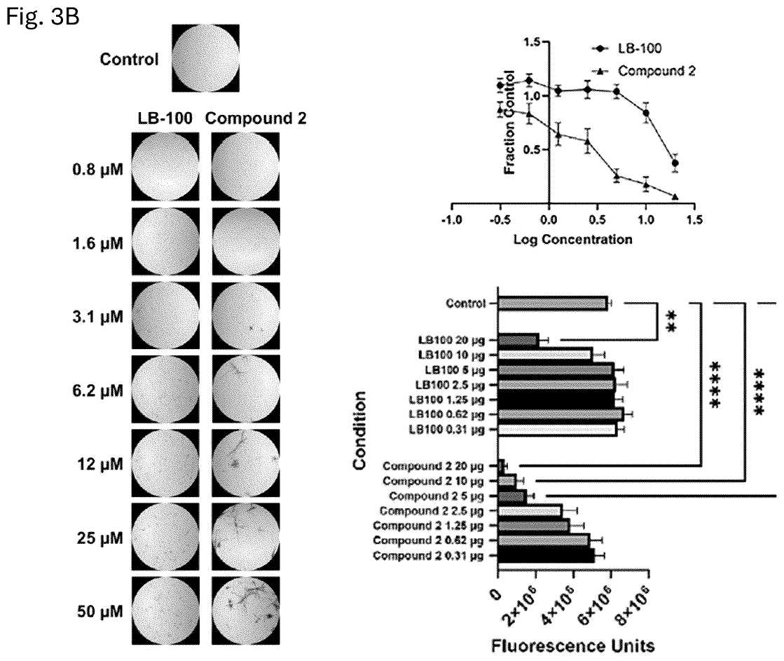

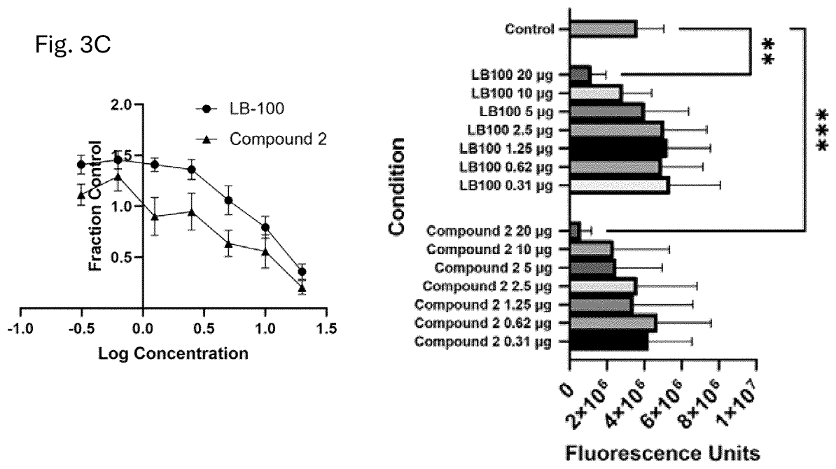

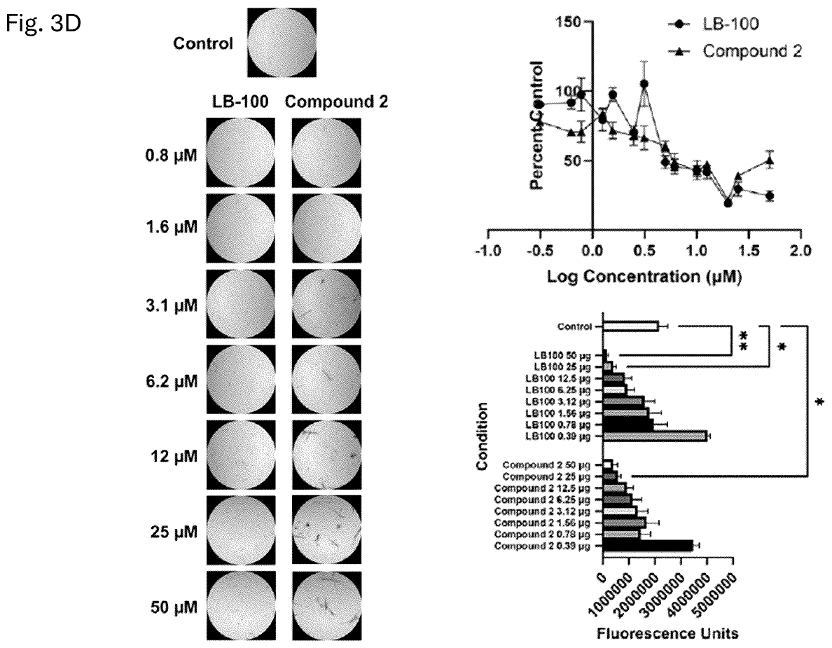

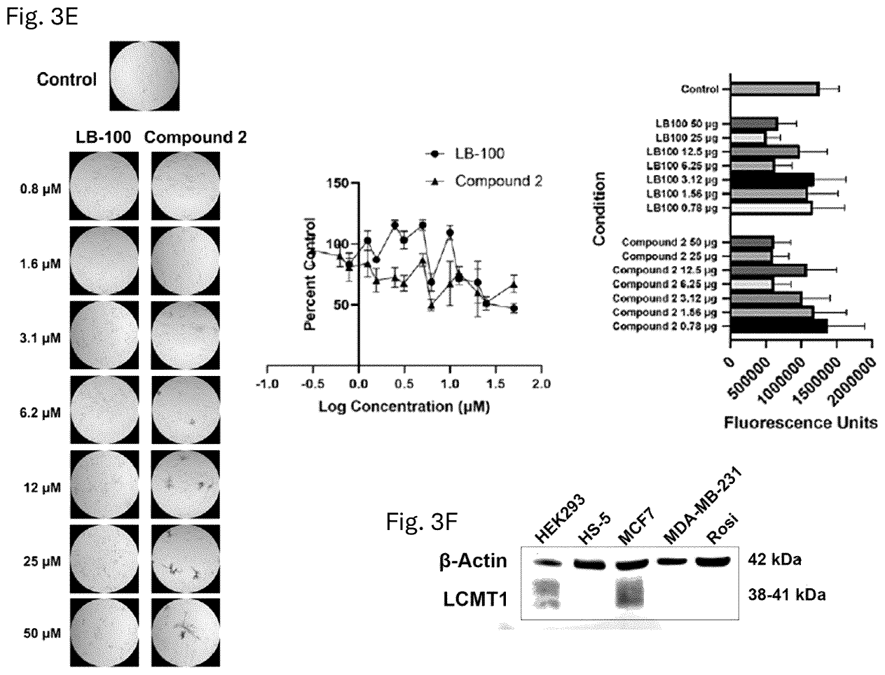

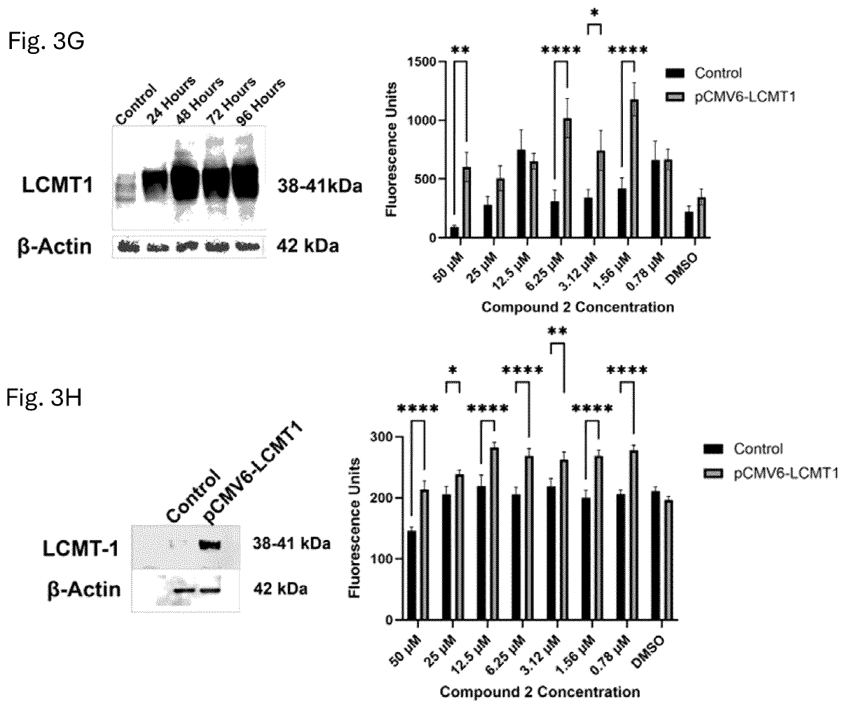

Compound 2 significantly inhibits survival of HEK-293, HS-5, MDA-MB-231, and Rosi cells, but not MCF7 cells. Because LCMT-1 knock-out gene editing is associated with lethality in embryos (MacKay et al. PloS one. 2013; 8(6):e65967), the effect of LCMT-1 inhibition with Compound 2 on survival was tested. Cytotoxicity assays revealed that Compound 2 inhibited survival of HEK-293 and Rosi cells at concentrations between 5 and 20 μM (p=0.0192 at 5 μM, p=0.0014 at 10 μM, and p=0.0003 at 20 μM for HEK-293 cells; p=0.0003 at 5 μM, p<0.0001 at 10 μM and 20 μM for Rosi cells), while LB-100 inhibited survival at 20 μM (p=0.0036 for HEK-293 cells, p=0.0035 for Rosi cells, FIGS. 3A and 3B). In HS-5 cells, Compound 2 cytotoxicity at 20 μM (p=0.0004) was comparable to that of LB-100 at 20 μM (p=0.0087, FIG. 3C). In MDA-MB-231 cells, LB-100 inhibited survival in concentrations between 25 and 50 μM (p=0.0117 at 25 μM and p=0.0023 at 50 μM), while Compound 2 significantly inhibited survival at 25 μM (p=0.0425, FIG. 3D). Neither LB-100 nor Compound 2 significantly inhibited survival in MCF7 cells at the concentrations tested (p=0.3204, FIG. 3E). However, all cultures treated with Compound 2 contained necrotic figures not seen in cultures treated with LB-100. Half maximal effective concentrations of Compound 2 were lower than that of LB-100 for all cell lines tested:

TABLE 1
Half maximal effective concentrations of Compound 2 were
lower than that of LB-100 for all cell lines tested.

Log [μM] EC₅₀

| Cell Line | LB-100 | Compound 2 |
|---|---|---|
| HEK-293 | 1.33 | 0.67 |
| HS-5 | 1.56 | 0.94 |
| MCF7 | 1.66 | 0.35 |
| MDA-MB-231 | 0.83 | 0.59 |
| Rosi | 4.34 | 0.50 |

LCMT-1 overexpression confers resistance to Compound 2-mediated inhibition of cell survival. Baseline expression of LCMT-1 was assessed in the HEK-293, HS-5, MCF7, MDA-MB-231 and Rosi cell lines. This revealed relative LCMT-1 overexpression in HEK-293 and MCF7 cells (FIG. 3F). LCMT-1 was overexpressed in HEK-293 and MDA-MB-231 cells with the pCMV6-LCMT1 mammalian plasmid vector (FIGS. 3F and 3H). For HEK-293 cells, only the cells treated with 50 μM Compound 2 had decreased survival as compared to cells treated with other concentrations of Compound 2 (p=0.0001), while there was decreased survival in the HEK-293^{pCMV6-LCMT1} cells at 50 and 25 μM concentrations. LCMT-1 overexpression significantly increased survival of HEK-293 cells in the presence of Compound 2 (p=0.0006).

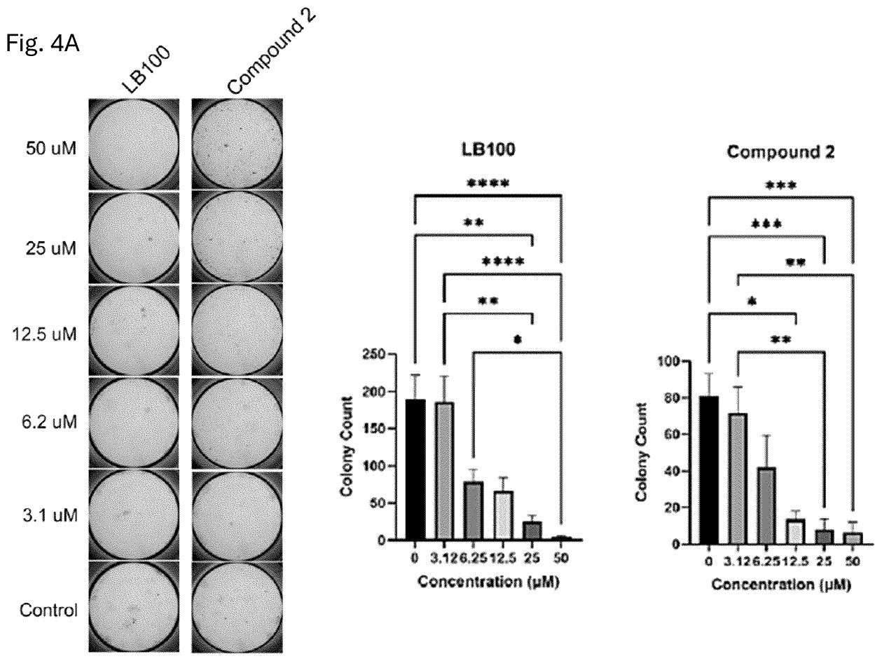

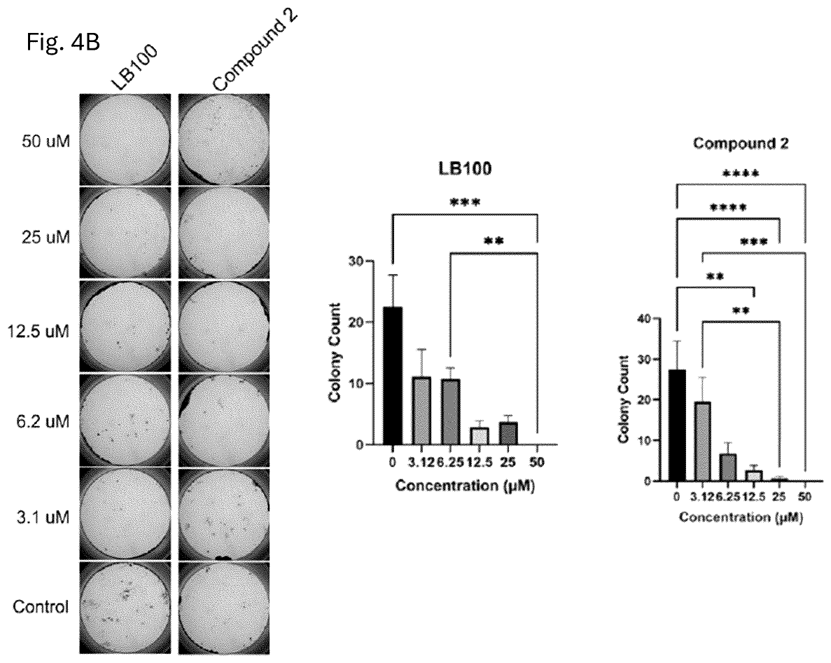

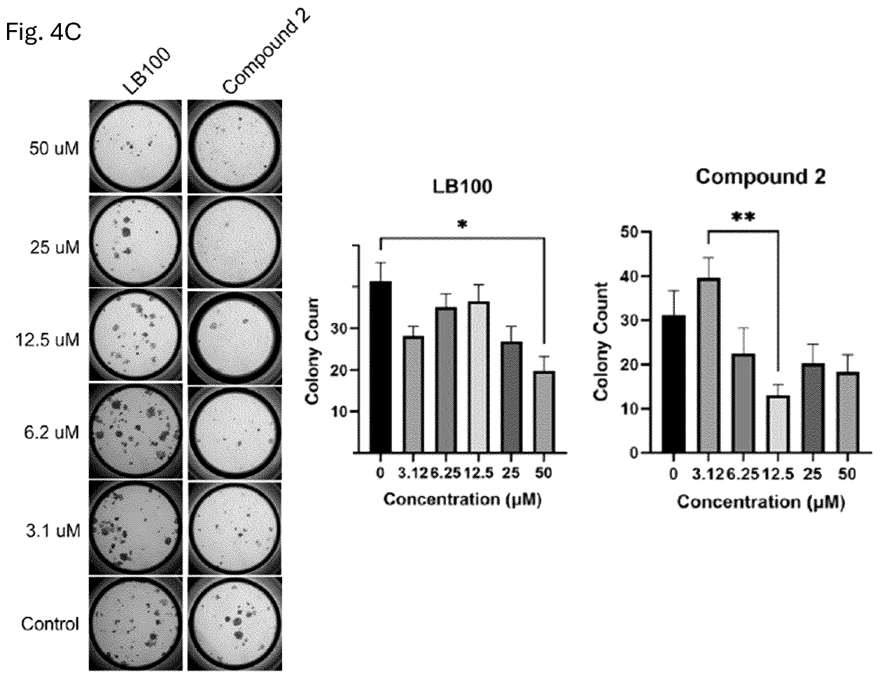

Compound 2 inhibits clonogenic colony formation in cancer cells. Silencing of PR55α inhibits clonogenic colony formation in pancreatic cancer cells (Hein et al. Oncogenesis. 2019; 8(11):63; Hein et al. Cancer research. 2016; 76(8):2243-53) and breast cancer cells (Di Conza et al. Cell Rep. 2017; 18(12):2836-44). The effect of prevention of PR55α binding to the PP2A holoenzyme through LCMT-1 inhibition was determined. Both LB-100 and Compound 2 inhibited colony formation in MDA-MB-231 (p<0.0001 for both compounds) and Rosi (p<0.0001 for both compounds) cells in a concentration-dependent manner (FIGS. 4A and 4B). Colony formation in MCF7 cells was inhibited to a lesser extent (p=0.0012 for LB-100, and p=0.0035 for Compound 2, FIG. 4C).

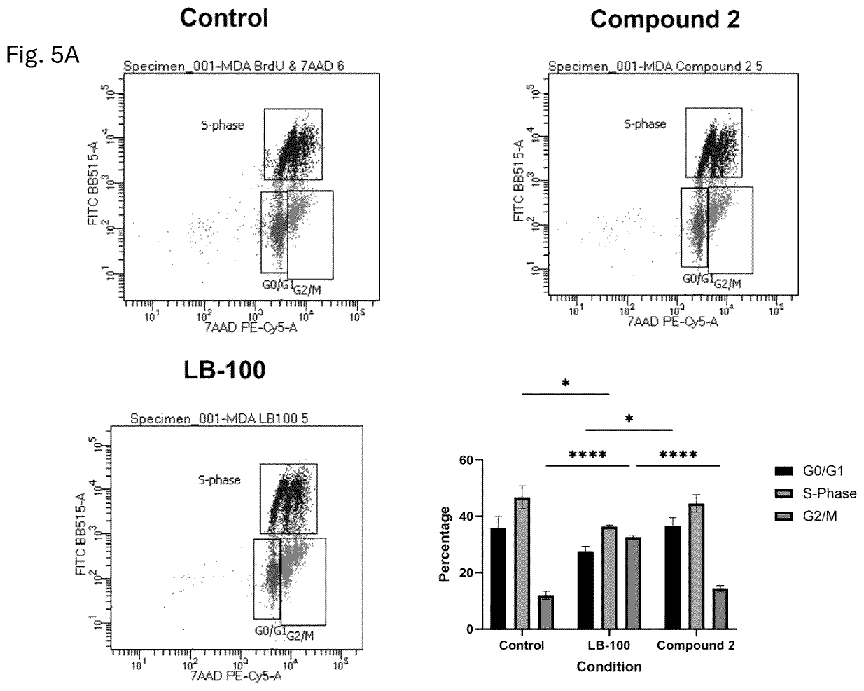

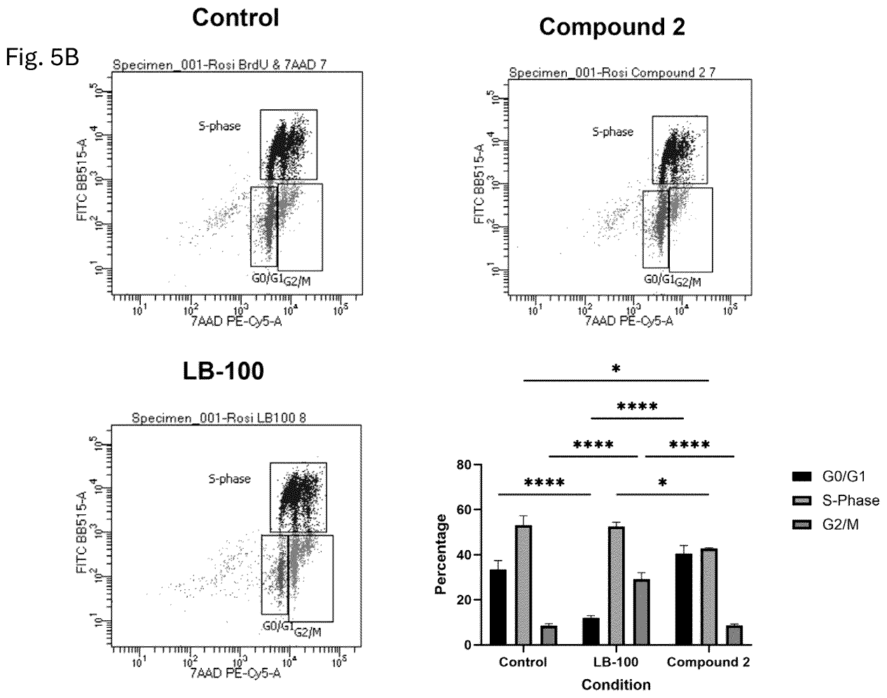

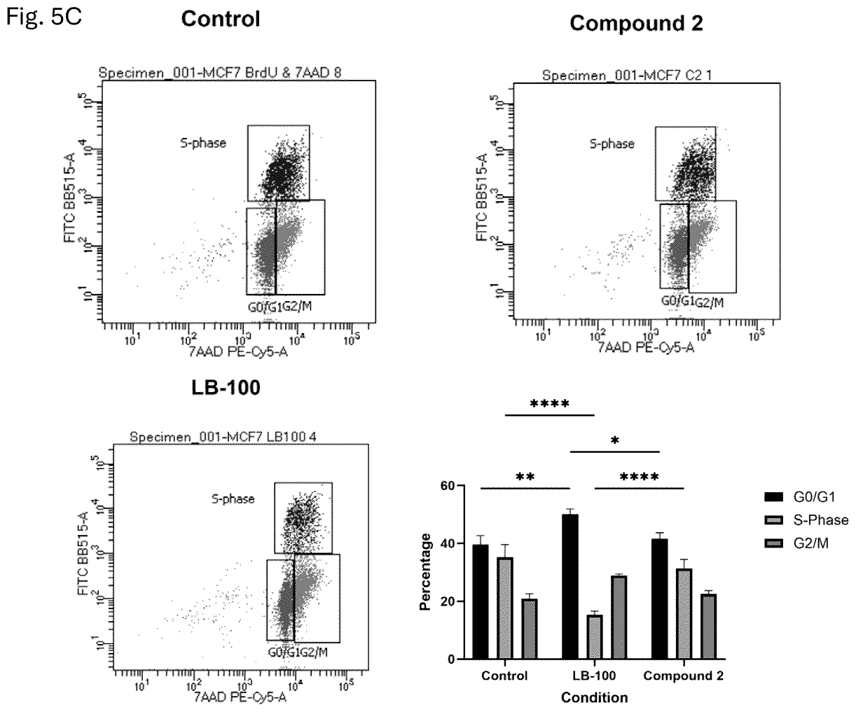

Compound 2 inhibits mitosis in Rosi cells. LCMT-1 plays an essential role in normal progression through mitosis (Lee JA. Et al., 2007, 282(42):30974-84; Stanevich V. et al., 2011, Mol Cell., 41(3):331-42; Zhang N. et al., 2023, 27:101572). LB-100 has been shown to induce cell cycle arrest in colorectal cancer cells (Dai C. et al., 2017, Oncotarget, 8(56):95810-23). To determine whether Compound 2 effects on cell survival and colony formation might be due to mitotic interference, cell cycle analysis was performed with BrdU flow cytometry. While LB-100 induced G2/M-phase arrest in MDA-MB-231 and Rosi cells (p<0.0001 for both comparisons), Compound 2 induced G0/G1 arrest only in Rosi cells (p=0.0193, FIGS. 5A and 5B). LB-100 induced G0/G1 arrest MCF7 cells (p=0.0063 for G0/G1 and p<0.0001 for S-phase).

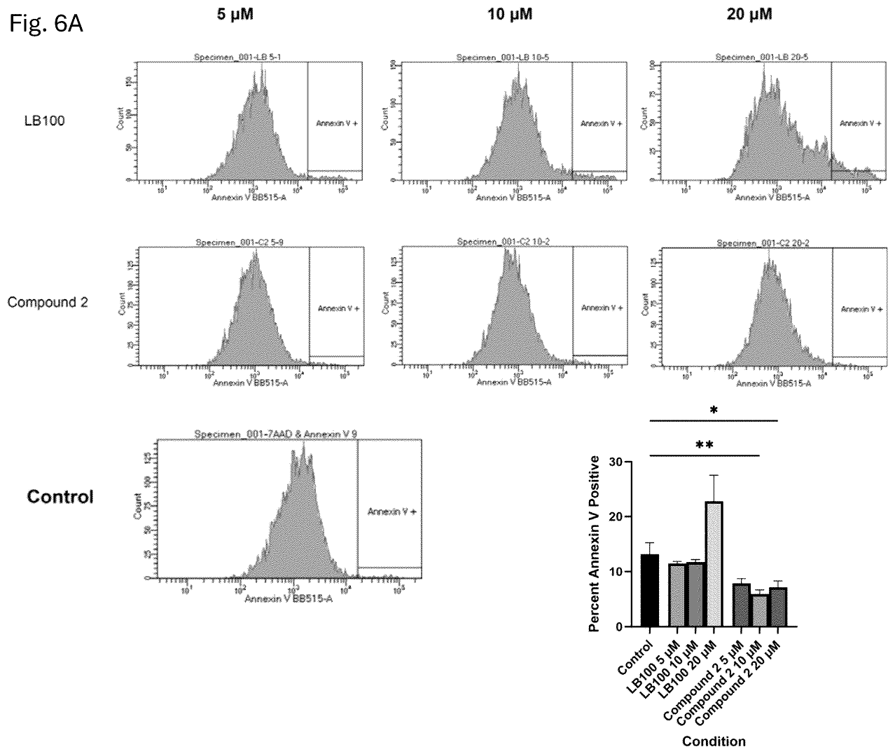

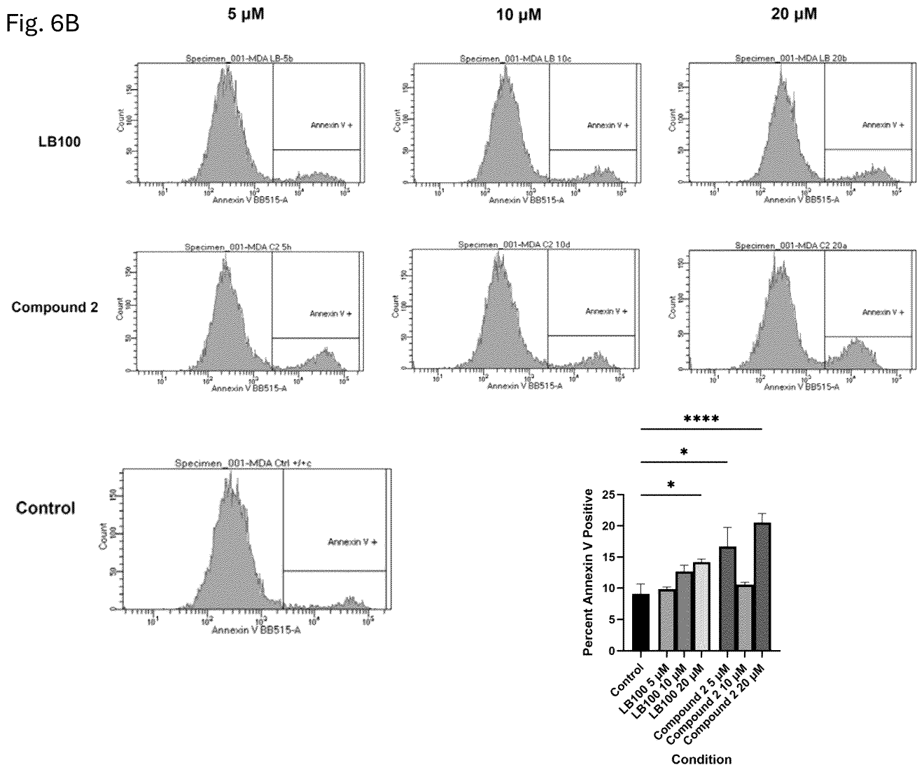

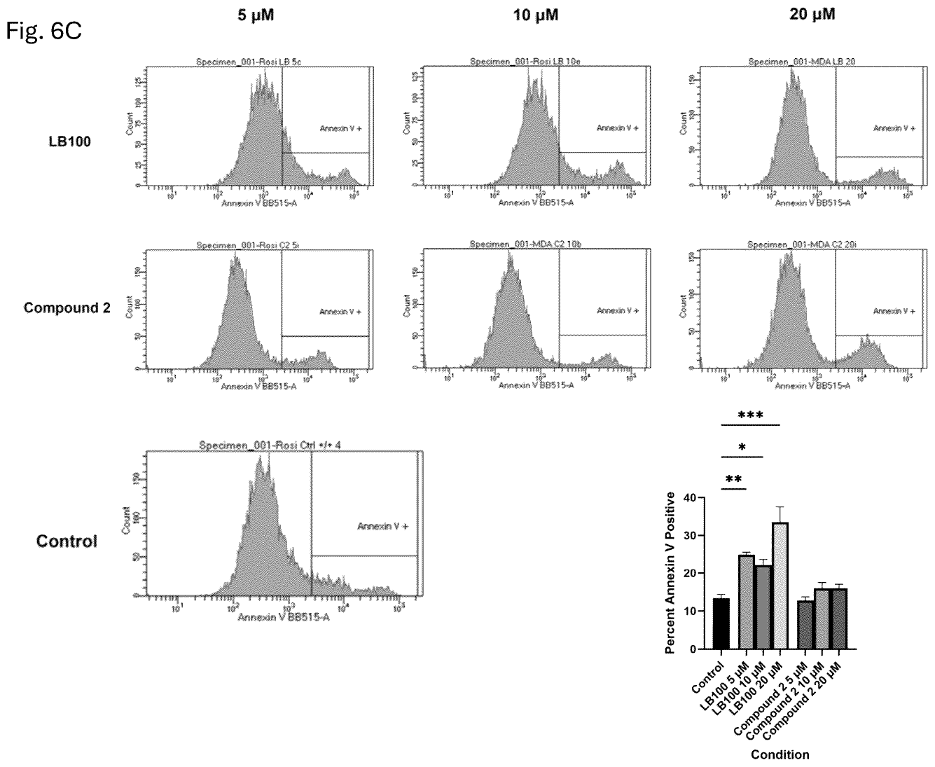

Compound 2 induces apoptosis in MDA-MB-231 cells, but not in MCF7 nor Rosi cells. Silencing of LCMT-1 (Longin et al. The Journal of biological chemistry. 2007; 282(37):26971-80; Lee et al. The Journal of biological chemistry. 2007; 282(42):30974-84) and PR55α (Di Conza et al. Cell Rep. 2017; 18(12):2836-44; Lee et al. The Journal of biological chemistry. 2007; 282(42):30974-84) result in apoptosis, and LB-100 has been shown to induce apoptosis in colorectal cancer cells (Dai et al. Oncotarget. 2017; 8(56):95810-23). To determine whether the results of the cell survival and clonogenic assays represented apoptosis induced by LB-100 and Compound 2, annexin V assays were performed. Treatment with Compound 2 resulted in increased cell surface annexin V expression in MDA-MB-231 cells (p<0.0001 at 20 μM), and in decreased cell surface annexin V in MCF7 cells (p=0.0013 at 10 μM and p=0.0397 at 20 μM). Compound 2 did not induce apoptosis in Rosi cells (p>0.9999 at 5 μM, p=0.6764 at 10 μM and p=0.3002 at 20 μM). LB-100 induced apoptosis in MDA-MB-231 (p=0.0258 at 20 μM) and Rosi cells (p=0.0019 at 5 μM, p=0.0126 at 10 μM, and p=0.0007 at 20 μM, FIG. 6).

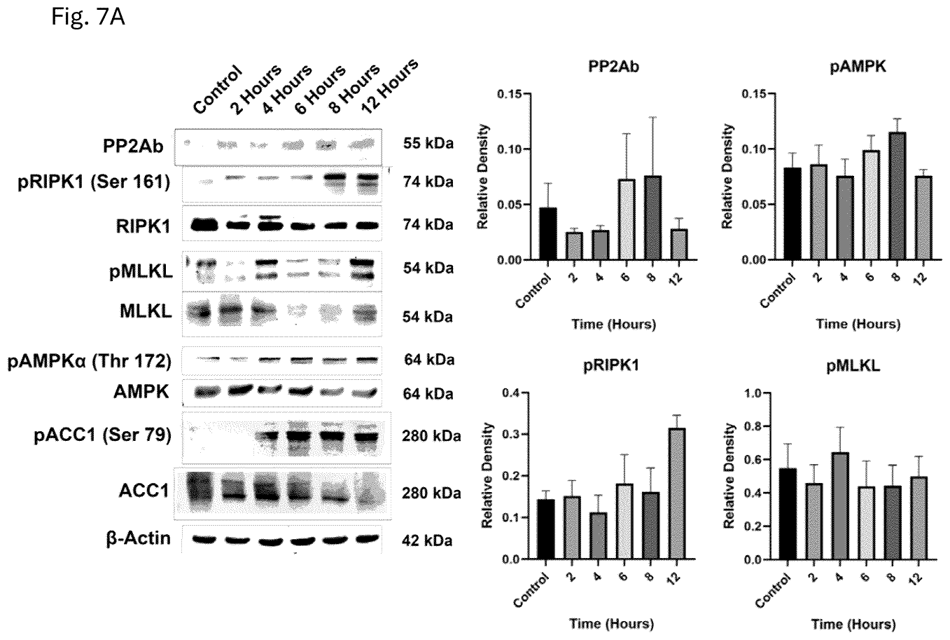

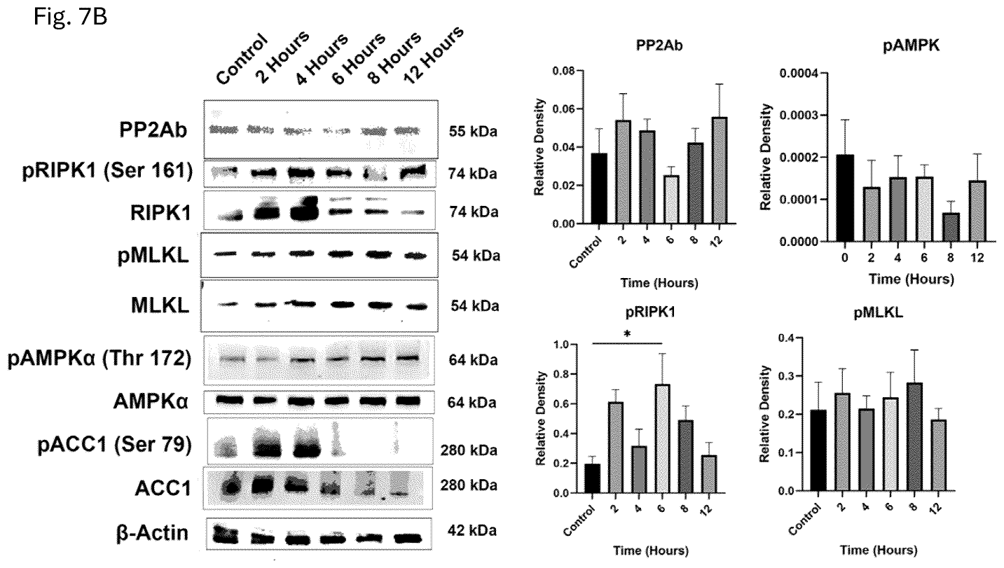

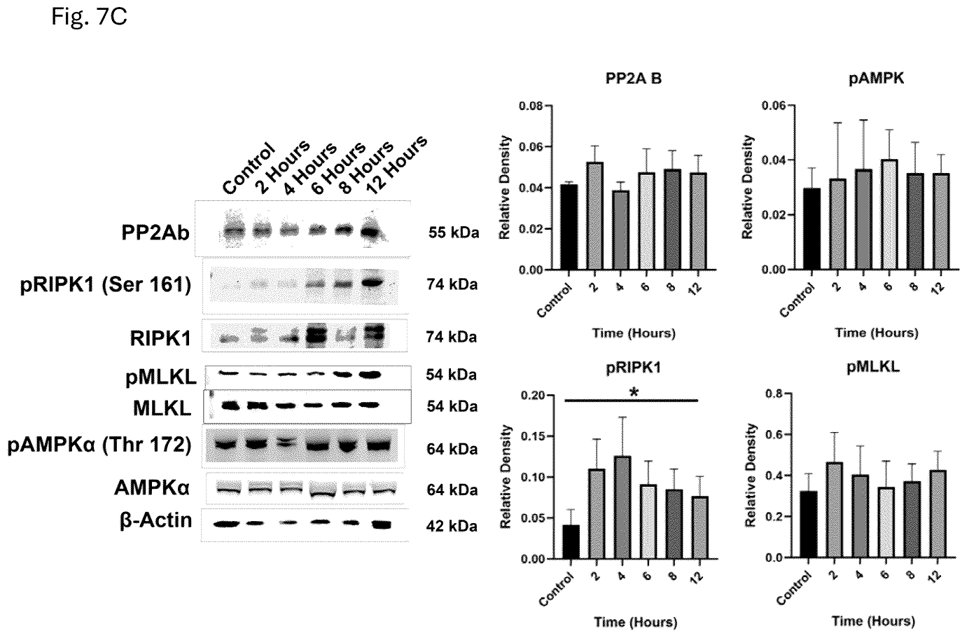

Compound 2 induces RIPK1 phosphorylation in cancer cells. Glucose deprivation has been shown to induce cell death in some cell lines through calcium-dependent PP2Ac demethylation, and this form of cell death is mediated by RIPK1 phosphorylation (Lee et al. Science signaling. 2018; 11(512)). It was determined that treatment with compound 2 resulted in increased RIPK1 phosphorylation at serine 161 in the cancer cell lines, but this was statistically significant only for MDA-MB-231 (p=0.0273) and Rosi cells (p=0.0179, FIG. 7). Phosphorylation of RIPK1 in the presence of Compound 2 did not statistically increase in MCF7 cells (p=0.0598). None of the cell lines demonstrated RIPK1 phosphorylation at serine 166. Dai et al reported that treatment of colorectal cancer cells with LB-100 induced phosphorylation of AMPK (Dai et al. Oncotarget. 2017; 8(56):95810-23). Increased phosphorylation of AMPK was not found in tested cell lines in response to treatment with Compound 2 (p=0.0906 for MCF7 cells, p=0.0660 for MDA-MB-231 cells, and p=0.6775 for Rosi cells). However, treatment with Compound 2 increased phosphorylation of ACC1 in MCF7 and Rosi cells.

Dysregulation of the delicate balance between kinases and phosphatases characteristic of most cancers is complex, and can be mediated by gain of function mutations in kinases, loss of function mutations in phosphatases, overexpression of microRNAs that target phosphatases, deletions or mutations in genes that activate phosphatases, or overexpression of oncogenes that inactivate phosphatases (Remmerie et al. Frontiers in oncology. 2019; 9:462; Ivovic et al. Neoplasma. 2023; 70(4):485-99; Johnson et al. Cancer Cell Int. 2024; 24(1):11). Most therapies aimed at restoring this balance have targeted kinases, in part because they were discovered before the phosphatases, and because the importance of the phosphatases has been discovered relatively recently (Turdo et al. Front Cell Dev Biol. 2021; 9:690306). PP2A is a major serine/threonine phosphatase, and several direct and indirect PP2A activators have shown therapeutic efficacy for a variety of malignancies.

Direct activators of PP2A include phenothiazines and their analogues, metformin, sodium selenate, forskolin and NSC49L (Johnson et al. Cancer Cell Int. 2024; 24(1):11). These molecules are thought to exert their effects by stabilizing the PP2A heterotrimer (Leonard et al. Cell. 2020; 181(3):688-701 e16; Farrington et al. The Journal of biological chemistry. 2020; 295(3):757-70). Indirect activators of PP2A target endogenous PP2A inhibitors such as PME-1, Suvar/Enhancer of zeste/Trithorax (SET), two A inhibitory protein (TIPRL1), α-endosulfine, ARPP-16, ARPP-19 and cancerous inhibitor of PP2A (CIP2A) (10, 44). These include FTY720, CM-1231, OSU-25, OP449, Bortezomib, ethoxysanguarine, TD52, actigenin, celastrol, niclosamide, polyphyllin I and Lapatinib (Johnson et al. Cancer Cell Int. 2024; 24(1):11).

However, few PP2A activators have advanced to clinical trials due to their side effect profiles and also because PP2A plays a complicated role in oncogenic signaling, behaving as both a tumor suppressor and promoter. PP2A downstream effects are mediated by substrate specificity, which is in turn dependent upon the heterotrimeric composition. While deletions of the gene for the B55α subunit have been detected in breast (Beca et al. BMC cancer. 2015; 15:285) and prostate (Zhao et al. Oncogenesis. 2019; 8(12):72; Cheng et al. Cancer Genet. 2011; 204(7):375-81) cancers, PP2A heterotrimers with the B55α subunit have been shown to promote the transcription of Wnt-responsive genes such as cyclin D1 and c-Myc through dephosphorylation of β-catenin (Wlodarchak et al. Crit Rev Biochem Mol Biol. 2016; 51(3):162-84; Pippa et al. Cells. 2020; 9(3)). PP2A-B55α also activates mitogen activated protein kinase signaling through dephosphorylation of the kinase suppressor of Ras 1 and Raf-1 (Ory et al. Curr Biol. 2003; 13(16):1356-64). The B55α subunit activates the HPV16 long control region in cells with a deletion in the short arm of chromosome 11 (Smits et al. The EMBO journal. 1992; 11(12):4601-6), sustains oncogenic signaling in pancreatic cancer cells (Hein et al. Oncogenesis. 2019; 8(11):63; Hein et al. Cancer research. 2016; 76(8):2243-53), and mediates resistance to glucose-starvation-mediated cell death in breast cancer cells (Di Conza et al. Cell Rep. 2017; 18(12):2836-44). Thus, different mechanisms of PP2A dysregulation are dependent upon the cellular genetic, epigenetic and metabolic environment.

The rationale for PP2A inhibition is based on the role that PP2A, including PP2A-B55α, plays in the DNA damage response (Kalev et al. Cancer research. 2012; 72(24):6414-24; Mazhar et al. Biochim Biophys Acta Mol Cell Res. 2019; 1866(1):51-63; Dohoney et al. Oncogene. 2004; 23(1):49-57; Li et al. The EMBO journal. 2007; 26(2):402-11; Li et al. Toxicology. 2015; 331:57-65; Chowdhury et al. Mol Cell. 2005; 20(5):801-9). PP2A inhibitors allow cells with damaged DNA to enter the cell cycle, resulting in cytotoxicity due to unstable chromatin (Johnson et al. Cancer Cell Int. 2024; 24(1):11; Mazhar et al. Biochim Biophys Acta Mol Cell Res. 2019; 1866(1):51-63; Li et al. Toxicology. 2015; 331:57-65). Several PP2A inhibitors (okadaic acid, calyculin A, tautomycin, tautomycetin, fostriecin, cantharidin, noncantharidein, and LB-100) have shown efficacy against a variety of cancer cells, but only LB-100, a cantharidin derivative, has advanced to clinical trials (Chung et al. Clinical cancer research: an official journal of the American Association for Cancer Research. 2017; 23(13):3277-84) due to toxicities with other PP2A inhibitors (Johnson et al. Cancer Cell Int. 2024; 24(1):11). LB-100 restricts tumor growth in melanoma (Hu et al. Pathology oncology research: POR. 2022; 28:1610572), colorectal (Dai et al. Oncotarget. 2017; 8(56):95810-23), hepatocellular (Sun et al. Cell Death Dis. 2021; 12(9):849), and breast (Uddin et al. MH, Cell Cycle. 2020; 19(5):592-600) cancer cells. LB-100 has shown promise as a sensitizer for DNA-damaging therapies (chemotherapy and radiotherapy) (Ronk et al. Cancer Biol Med. 2022; 19(10):1428-39; Hong et al. Cancer Biol Ther. 2015; 16(6):821-33; Lv et al. Oncotarget. 2014; 5(17):7512-24; Wei et al. Clinical cancer research: an official journal of the American Association for Cancer Research. 2013; 19(16):4422-32; Ho et al. Cancer letters. 2018; 415:217-26; Song et al. Cancers (Basel). 2021; 13(19); Bai et al. Cancer letters. 2014; 355(2):281-7; Bai et al. Molecular cancer therapeutics. 2014; 13(8):2062-72; Gao et al. Cell Oncol (Dordr). 2022; 45(6):1203-15; Hao et al. Neuro Oncol. 2018; 20(6):799-809; Hu et al. Sci Rep. 2017; 7(1):2894; Chang et al. Molecular cancer therapeutics. 2015; 14(1):90-100; Ho et al. Oncotarget. 2016; 7(11):12447-63; Gordon et al. Molecular cancer therapeutics. 2015; 14(7):1540-7; Zhang et al. Cell Cycle. 2015; 14(13):2100-8), as well as an enhancer of chimeric antigen receptor T-cell therapy (Cui et al. Cancers (Basel). 2020; 12(1)), and immune checkpoint inhibition (Maggio et al. J Neurooncol. 2020; 148(2):231-44; Ho et al. Nat Commun. 2018; 9(1):2126; Mirzapoiazova et al. Molecular cancer therapeutics. 2021; 20(10):1820-35).

Methylation of the PP2Ac catalytic subunit at leucine 309 near it carboxyl terminus by LCMT-1 stabilizes binding of the B55α subunit to the PP2A heterotrimer (Longin et al. The Journal of biological chemistry. 2007; 282(37):26971-80). While LCMT-1 silencing promotes castration-resistant prostate cancer growth through increasing androgen receptor activity (Rasool et al. Nat Commun. 2023; 14(1):5253), LCMT-1 overexpression carries a poor prognosis in hepatocellular carcinoma (Zhang et al. Translational oncology. 2023; 27:101572). Because LCMT-1 is necessary for progression through mitosis, it has been identified as an oncologic target (Longin et al. The Journal of biological chemistry. 2007; 282(37):26971-80; Lee et al. The Journal of biological chemistry. 2007; 282(42):30974-84; Zhang et al. Translational oncology. 2023; 27:101572).

Neither of the compounds studied as potential LCMT-1 inhibitors were analogues of SAM, nor were they PP2Ac peptidomimetics. While there are limitations of molecular modelling (Limongelli et al. Wiley Interdisciplinary Reviews-Computational Molecular Science. 2020; 10(4)), the models described herein indicate that these compounds did not interact with LCMT-1 at the SAM or PP2Ac carboxyl terminus binding sites (FIGS. 1B and 1C). Previous attempts at LCMT-1 inhibition using synthetic peptides based on the carboxyl terminus of PP2Ac revealed that these peptides were neither substrates nor inhibitors of LCMT-1, suggesting that LCMT-1 recognizes aspects of the tertiary and/or quaternary structure of PP2Ac (Xie et al. The Journal of biological chemistry. 1994; 269(3):1981-4). Binding of Compound 2 to LCMT-1 may induce or prevent conformational changes in LCMT-1 that restrict binding or methylation of the PP2Ac carboxyl terminus, and this will have to be confirmed with x-ray crystallography. This could be consistent with the work of Stanevich et al, who determined that the conformation of the LCMT-1 active site pocket changes to allow binding of the PP2Ac carboxyl terminus (Stanevich et al. Mol Cell. 2011; 41(3):331-42).

Inhibition of LCMT-1 by Compound 2 had differential effects on the cell cycle (FIG. 5), survival (FIG. 3A-E) and induction of apoptosis (FIG. 6) in the cell lines tested, but Compound 2 inhibited clonogenic colony formation in all three of the cancer cell lines (FIG. 4). The observed variability in response to Compound 2 among the cell lines may in part be explained by differences in baseline expression of LCMT-1, which was increased in HEK-293 and MCF7 cells (FIG. 3f), and could explain their relative resistance to Compound 2. However, HS-5 cells demonstrated a similar response to Compound 2 as did HEK-293 cells without overexpression of LCMT-1. Though overexpression of LCMT-1 in HEK-293 and MDA-MB-231 cells increased resistance to Compound 2-mediated toxicity (FIGS. 3G and 3H), Compound 2 did not induce statistically significant PP2Ac demethylation in MCF7 cells (FIG. 2d). This may be indicative of off-target effects of Compound 2 since molecular modelling indicated that Compound 2 may dock at the SAM binding site of the SET domain of MLL5, and partially dock at the SAM binding site of the ARMT1 α chain. Compound 2 also partially docked with the ARMT1β chain (FIGS. 11-13 Relatively high concentrations of Compound 2 were needed to obtain cellular effects, which could also be indicative of off-target effects. However, this was also true of LB-100, and Compound 2 was more effective at inhibiting cell survival than was LB-100 in the cell lines tested (FIG. 3A-D).

Compound 2 decreased cytosolic PP2Ab expression in MCF7 and Rosi cells after 4-6 hours of exposure (FIG. 7). This is consistent with a previous study demonstrating that PP2Ac demethylation induced by oxidative stress results in increased B55α subunit binding to PME-1 and translocation to the nucleus in glioblastoma cells after 4 hours. PP2Ac demethylation resulted in increased phosphorylation of RIPK1 (Guffens et al. Cell Death Discov. 2023; 9(1):265). Data indicate that cytosolic localization of the B55α subunit rebounded after 8 hours.

Phosphorylation of RIPK1 is increased at serine 161 in response to treatment with Compound 2 in the tested cell lines. While activation of both RIPK1 and AMPK pathways are linked to depletion of cellular adenosine triphosphate, activation of the AMPK pathway has been shown to prevent formation of the RIPK1-RIPK3 necroptosis complex (Lee et al. Nat Cell Biol. 2019; 21(8):940-51; Zhang et al. Science. 2023; 380(6652):1372-80). Nevertheless, RIPK1 has been shown to communicate energetic stress to AMPK through the mechanistic target of rapamycin complex 1 (Najafov et al. Mol Cell. 2021; 81(2):370-85 e7). Though phosphorylation of AMPKα by Compound 2 is not statistically significant in the tested cell lines, Compound 2 increased phosphorylation of AMPKα and ACC1 in MCF7 and Rosi cells. AMPK is the main kinase regulator of ACC1, inactivating ACC1 when cellular energy stores are low. Increased phosphorylation of MLKL was noted in Rosi and MCF7 cells, but not in MDA-MB-231 cells. This suggests that while necroptosis may have been the mechanism of cell death in Rosi and MCF7 cells, a different mechanism occurred in MDA-MB-231 cells. This is consistent with FIG. 6, which indicates that Compound 2 only induced apoptosis in MDA-MB-231 cells. Though increased RIPK1 phosphorylation was evident in MDA-MB-231 cells in response to Compound 2, RIPK1 can also contribute to apoptosis (Kaiser et al. Proceedings of the National Academy of Sciences of the United States of America. 2014; 111(21):7753-8).

PP2A inhibition has been shown to impact other disease processes including nonalcoholic fatty liver disease (Chen et al. World J Gastroenterol. 2019; 25(45):6607-18), ultraviolet radiation-induced damage in retinal pigment epithelium (Li et al. Biochemical and biophysical research communications. 2018; 506(1):73-80), depression (Lecca et al. Nature medicine. 2016; 22(3):254-61), renal and hepatic fibrosis (Nyamsuren et al. Sci Rep. 2021; 11(1):24075), and cardiovascular disease (Zhang et al. Eur J Pharmacol. 2019; 852:99-106). The role that LCMT-1 may play in these disease processes has not been studied.

In conclusion, methyl 4-methyl-2-[(2-methylbenzoyl)amino]-5-[[(3-methylphenyl)amino]carbonyl]-3-thiophenecarboxylate (Compound 2) is an LCMT-1 inhibitor that may have anti-tumor effects including inhibition of survival, induction of cell cycle arrest and apoptosis in some cells. Compound 2 inhibits clonogenic colony formation, and these effects seem to be mediated by necroptosis through demethylation of PP2Ac in some cells, and apoptosis in others. Further studies are needed to confirm Compound 2 binding to LCMT-1 and potential off-target effects.

### Example 2: a Small Molecule Leucine Carboxyl Methyltransferase Inhibitor Sensitizes Cancer Cells to Irradiation

Addressing Signaling Dysregulation with PP2A Activation.

Approximately two thirds of proteins undergo phosphorylation (Ardito, F. et al., 2017, Int J Mol Med 40(2): 271-280). Serine phosphorylation is the most common event, followed by threonine phosphorylation, with tyrosine phosphorylation being the rarest (Sacco, F. et al., 2012, FEBS Lett 586(17): 2732-2739). Several direct and indirect PP2A activators have shown preclinical therapeutic efficacy for a variety of malignancies (Johnson, H., S. Narayan and A. K. Sharma, 2024, Cancer Cell Int 24(1): 11). Direct PP2A activators may stabilize the PP2A heterotrimer (Farrington, C. et al., 2020, J Biol Chem 295(3): 757-770). Indirect activators target endogenous PP2A inhibitors such as PME-1, Suvar/Enhancer of zeste/Trithorax (SET), two A inhibitory protein (TIPRL1), α-endosulfine, ARPP-16, ARPP-19 and cancerous inhibitor of PP2A (CIP2A) (Haesen, D. et al., 2014, Front Oncol 4: 347; Johnson, H., S. Narayan and A. K. Sharma, 2024, Cancer Cell Int 24(1): 11). However, few PP2A activators have advanced to clinical trials due to side effect profiles, and because PP2A behaves as both a tumor suppressor and promoter.

### Role of PR55α in Oncogenesis.

While the B55α subunit gene is deleted in some breast (Beca, F. et al., 2015, BMC Cancer 15: 285) and prostate (Cheng, Y. et al., 2011, Cancer Genet 204(7): 375-381; Zhao, Z. et al., 2019, Oncogenesis 8(12): 72) cancers, PP2A enzymes with the B55α subunit promote transcription of Wnt-responsive genes such as cyclin D1 and c-Myc through dephosphorylation of β-catenin (Wlodarchak, N. and Y. Xing, 2016, Crit Rev Biochem Mol Biol 51(3): 162-184; Pippa, R. and M. D. Odero, 2020, Cells 9(3)). PP2A-B55α activates mitogen activated protein kinase signaling through dephosphorylation of the kinase suppressor of Ras 1 and Raf-1 (Ory, S. et al., 2003, Curr Biol 13(16): 1356-1364). PP2A-B55α activates the human papilloma virus-16 long control region in cells with deletion of the short arm of chromosome 11 (Smits, P. et al., 1992, EMBO J 11(12): 4601-4606.), sustains oncogenic signaling in pancreatic cancer (Hein, A. et al., 2019, Oncogenesis 8(11): 63; Hein, A. et al., 2016, Cancer Res 76(8): 2243-2253), and facilitates resistance to glucose-starvation-mediated cell death in breast cancer (Di Conza, G. et al., 2017, Cell Rep 18(12): 2836-2844). Thus, differential effects of PP2A dysregulation are dependent upon the cellular genetic, epigenetic and metabolic environments.

### PP2A and the DNA Damage Response.

Resistance to DNA-damaging therapies (chemotherapy and radiation therapy) is common in cancer patients (Jurkovicova, D. et al., 2022, Int J Mol Sci 23(23)). This resistance is mediated by an augmented deoxyribonucleic acid (DNA) damage response (DDR). The rationale for PP2A inhibition is based on the role that PP2A, including PP2A-B55α, plays in the DDR (Dohoney, K. et al., 2004, Oncogene 23(1): 49-57; Chowdhury, D. et al., 2005, Mol Cell 20(5): 801-809; Li, H. H. et al., 2007, EMBO J 26(2): 402-411; Kalev, P. et al., 2012, Cancer Res 72(24): 6414-6424; Li, X. et al., 2015, Toxicology 331: 57-65; Mazhar, S. et al., 2019, Biochim Biophys Acta Mol Cell Res 1866(1): 51-63). PP2A inhibitors allow cells with damaged DNA to enter the cell cycle, resulting in cytotoxicity due to unstable chromatin (Li, X. et al., 2015, Toxicology 331: 57-65; Mazhar, S. et al., 2019, Biochim Biophys Acta Mol Cell Res 1866(1): 51-63; Johnson, H. et al., 2024, Cancer Cell Int 24(1): 11). Several PP2A inhibitors (okadaic acid, calyculin A, tautomycin, tautomycetin, fostriecin, cantharidin, noncantharidein, and LB-100) have shown efficacy against a variety of cancer cells, but only LB-100, a cantharidin derivative, has advanced to clinical trials (Chung, V. et al., 2017, Clin Cancer Res 23(13): 3277-3284.) due to toxicities with other PP2A inhibitors (Johnson, H. et al., 2024, Cancer Cell Int 24(1): 11). LB-100 restricts tumor growth in melanoma (Hu, B. et al., 2022, Pathol Oncol Res 28: 1610572.), colorectal (Dai, C. et al., 2017, Oncotarget 8(56): 95810-95823), hepatocellular (Sun, B. et al., 2021, Cell Death Dis 12(9): 849.) and breast (Uddin, M. H. et al., 2020, Cell Cycle 19(5): 592-600) cancer cells. LB-100 has shown promise as a sensitizer for DNA-damaging therapies (Wei, D. et al., 2013, Clin Cancer Res 19(16): 4422-4432; Bai, X. et al., 2014, Cancer Lett 355(2): 281-287; Bai, X. et al., 2014, Mol Cancer Ther 13(8): 2062-2072; Lv, P. et al., 2014, Oncotarget 5(17): 7512-7524; Chang, K. E. et al., 2015, Mol Cancer Ther 14(1): 90-100; Gordon, I. K. et al., 2015, Mol Cancer Ther 14(7): 1540-1547; Hong, C. S., et al., 2015, Cancer Biol Ther 16(6): 821-833; Zhang, C. et al., 2015, Cell Cycle 14(13): 2100-2108; Ho, W. S. et al., 2016, Oncotarget 7(11): 12447-12463; Hu, C. et al., 2017, Sci Rep 7(1): 2894; Hao, S. et al., 2018, Neuro Oncol 20(6): 799-809; Ho, W. S. et al., 2018, Cancer Lett 415: 217-226; Song, Q. et al., 2021, Cancers (Basel) 13(19); Gao, S. et al., 2022, Cell Oncol (Dordr) 45(6): 1203-1215; Ronk, H. et al., 2022, Cancer Biol Med 19(10): 1428-1439), as well as an enhancer of chimeric antigen receptor T-cell therapy (Cui, J. et al., 2020, Cancers (Basel) 12(1)) and immune checkpoint inhibition (Ho, W. S. et al., 2018, Nat Commun 9(1): 2126; Maggio, D. et al., 2020, J Neurooncol 148(2): 231-244; Mirzapoiazova, T. et al., 2021, Mol Cancer Ther 20(10): 1820-1835).

### LCMT-1 as an Oncologic Target for Limiting PR55α Activity.

Identified as an oncotarget, LCMT-1 not only facilitates docking of the PP2A B55α subunit, but also drives glycolysis and is necessary for progression through mitosis (Lee, J. A. and D. C. Pallas, 2007, J Biol Chem 282(42): 30974-30984; Longin, S. et al., 2007, J Biol Chem 282(37): 26971-26980; Zhang, N. et al., 2023, Transl Oncol 27: 101572). LCMT-1 overexpression carries a poor prognosis in hepatocellular carcinoma (Zhang, N. et al., 2023, Transl Oncol 27: 101572). While LCMT-1 silencing promotes castration-resistant prostate cancer growth through increasing androgen receptor activity (Rasool, R. U. et al., 2023, Nat Commun 14(1): 5253), in other malignant cells LCMT-1 targeting results in apoptosis (Lee, J. A. and D. C. Pallas, 2007, J Biol Chem 282(42): 30974-30984; Longin, S. et al., 2007, J Biol Chem 282(37): 26971-26980).

This study identified a LCMT-1 inhibitor (3-Thiophenecarboxylic acid, 4-methyl-2-[(2-methylbenzoyl)amino]-5-[[(3-methylphenyl)amino]carbonyl]-, methyl ester (henceforth referred to as LCMT-li) through small molecule library screening. This study demonstrated that LCMT-li decreased PP2Ac methylation, decreased cytoplasmic expression of PP2Ab, increased phosphorylation of acetyl-coenzyme A carboxylase, induced cell cycle arrest in some cell lines, and prevented cell survival through necroptosis and apoptosis (cell line-dependent). This study also demonstrated that LCMT-1 overexpression mediated resistance to cell death (Arosarena, O. A. et al., 2024, Frontiers in Drug Discovery 4). This study sought to determine whether combining radiotherapy with LCMT-li promoted cell death and cell cycle arrest, and limited the DDR.

The materials and methods are now described.

### Cell Culture.

The human MDA-MB-231 and MCF7 breast carcinoma cell lines were obtained from the Fox Chase Cancer Center cell culture core facility. These cell lines have been previously shown to be sensitive to glucose starvation-mediated PR55α depletion (Di Conza, G. et al., 2017, Cell Rep 18(12): 2836-2844). SCC-25 human oral cancer cells were obtained from the American Type Culture Collection. UM-SCC-12, UM-SCC-14a, UM-SCC-49 and UM-SCC-69 human head and neck cancer cells were obtained from the Otolaryngology Department at the University of Michigan. SCC-15 and SCC-25 cells were grown in Dulbecco's modified Eagle's medium (DMEM) with Ham's F-12 (DMEM/F-12, Corning®, Glendale, Arizona, catalog #90-090-PB) with 10% fetal bovine serum (FBS, Corning® catalog #35-015-CV) and 400 ng/mL hydrocortisone (Sigma Chemical Company, Saint Louis, Missouri, catalog #H0135). The MDA-MB-231, MCF7, 501-MEL, Rosi, A549, UM-SCC-12, UM-SCC-14A, UM-SCC-49 and UM-SCC-69 cells were grown in DMEM (Corning® catalog #50-003-PC) with 10% FBS. Jurkat and THP-1 cells were grown in Roswell Park Memorial Institute 1640 medium (Corning® catalog #50-020-PC) with 10% FBS. Cells were maintained in a 5% carbon dioxide atmosphere at 37° C. and tested free of mycoplasma contamination. Cells were not maintained in culture for more than 6 months without cryopreservation.

### Cytotoxicity Assays.

MCF7, MDA-MB-231, 501-MEL, Rosi, A549, SCC-15, SCC-25, UM-SCC-12, UM-SCC-14a, UM-SCC-49 and UM-SCC69 cells were seeded in 96-well tissue culture plates (Corning, New York) at a density of 2.5×103 cells/well. After 24 hours, the media were changed to media with LCMT-li (ChemSpace, Monmouth Junction, New Jersey) at various concentrations (0.78-200 μM). Control cells were given fresh media. After 48 hours cell viability was assessed using CellTiter 96® AQeous Non-Radioactive Cell Proliferation Assays (Promega Corporation, Madison Wisconsin). Plates were read after 4 hours with a GloMax® Discover plate reader (Promega Corporation, Madison Wisconsin). The cytotoxicity assays were performed at least twice, each time with eight replicates per condition.

Jurkat and THP-1 cells were seeded in 6-well plates at a density of 2.5×105 cells/mL. After 24 hours LCMT-li was added at various concentrations. After 48 hours, cells were pelleted and washed twice with ice-cold phosphate-buffered saline (PBS). Cells were stained with Ghost Dye™ 710 (Cytek®, Bethesda, Maryland) viability dye, 0.1 μL/mL in PBS on ice in the dark for 30 minutes. Cells were pelleted and washed twice with ice-cold PBS with 10% FBS. Cells were pelleted and fixed with 4% formaldehyde in PBS on ice for 15 minutes. Cells were pelleted and resuspended in PBS with 5% bovine serum albumin (BSA). Cells were analyzed with a Becton-Dickinson FACSymphony™ flow cytometer with at least 3 replicates; 3×104 cells were analyzed with each replicate for statistical significance.

### Radiosensitization Assays.

MCF7, MDA-MB-231, 501-MEL, Rosi, A549, SCC-15, SCC-25, UM-SCC-12, UM-SCC-14a, UM-SCC-49 and UM-SCC69 cells were seeded in 96-well tissue culture plates as previously described. After 24 hours, the media were changed to media with LCMT-li at various concentrations as described. After 3 hours, cells were irradiated at doses listed in Table 2 with an RS-2000 X-Ray Irradiator (Rad Source Technologies, Buford, Georgia). Radiation doses were based on published radiosensitization studies. After 24 hours cell viability was assessed using crystal violet staining. Briefly, cells were washed with PBS, then stained with crystal violet solution (0.25 g crystal violet in 20% methanol) for 20 minutes with agitation. The wells were washed 6 times with PBS to remove excess crystal violet. Plates were allowed to dry for 48 hours, and then 200 μL methanol was added to each well. The plates were agitated for 20 minutes to dissolve the crystal violet, and absorbance was read at 560 nm. The

TABLE 2
Radiation Doses

| Cell Line | Radiation Dose (Grays) |
|---|---|
| 501-MEL | 8 (Otani, Naito et al., 2016, Sci Rep 6: |
| | 30689) |
| A549 | 4 (Kobayashi, Hiroyama et al., 2023, |
| | Biology (Basel) 12(11)) |
| Jurkat | 10 (Ardail, Maalouf et al., 2009, Int J |
| | Radiat Oncol Biol Phys 73(4): 1211- |
| | 1218) |
| MCF7 | 4 (Griffin and Marignol, 2018, Int J |
| | Radiat Biol 94(5): 472-477) |
| MDA-MB-231 | 10 (Shahrokh, Mansouri et al., 2019, J |
| | Lasers Med Sci 10(Suppl 1): S18-S22) |
| Rosi | 8 (Otani, Naito et al., 2016, Sci Rep 6: |
| | 30689) |
| SCC-15 | 8 |
| SCC-25 | 8 (Caney, Singh et al., 2004, Int J |
| | Radiat Biol 80(4): 291-299; Jank, |
| | Lenz et al., 2022, Invest New Drugs |
| | 40(3): 478-486) |
| THP-1 | 10 (Yoshino, Konno et al., 2018, Int J |
| | Mol Sci 19(10)) |
| UM-SCC-12 | 8 |
| UM-SCC-14a | 8 |
| UM-SCC-49 | 8 |
| UM-SCC-69 | 8 |

radiosensitization assays were performed twice, each time with eight replicates per condition.

Jurkat and THP-1 cells were seeded in 6-well plates as described. After 24 hours LCMT-li was added at various concentrations as described. After 3 hours, cells were irradiated at 10 Grays. After 24 hours, cell viability was assessed with Ghost Dye™ 710 staining and flow cytometry as described.

### Annexin V Assays.

Head and neck cancer (HNC) cell lines were used for further analyses. Cells were seeded in 60 mm dishes at a density of 8×105 cells/dish. After 24 hours, cells were treated with LCMT-li, radiotherapy or both. Control cells were given fresh media. Because radiosensitization was seen between 2.5-10 μM in SCC-15, SCC-25, UM-SCC-49, and UM-SCC-69, irradiated cells were treated with or without 10 μM LCMT-li as described. Twenty-four hours after treatment, cells were lifted with Accutase and washed twice with PBS (pH 7.4) with 10% FBS. Cells were resuspended in annexin V binding buffer (10 mM HEPES (pH 7.4), 140 mM NaCl and 2.5 mM CaCl₂)) and labeled with FITC-anti-Annexin V and 7-AAD (Biolegend, San Diego, California, FITC Annexin V Apoptosis Detection Kit with 7-AAD, catalog #640922). Cell surface annexin V expression was assessed immediately with flow cytometry. Annexin V assays were performed with 12 replicates, 104 cells/replicate.

### Western Blots.

To better understand cellular outcomes associated with combining LCMT-li with radiotherapy (Adjemian, S. et al., 2020, Cell Death Dis 11(11): 1003), SCC-15, SCC-25, UM-SCC-12, UM-SCC-14a and UM-SCC-49 cells were treated with LCMT-li, radiotherapy or both as described for Annexin V assays. UM-SCC-69 cells were not used for immunoblots because insufficient numbers of cells survived irradiation to obtain sufficient lysates. After 24 hours, cells were lysed in radioimmunoprecipitation assay buffer containing protease inhibitors (Boster Bio, Pleasanton, California, catalog #AR1182-1) and phosphatase inhibitors (Santa Cruz Biotechnology catalog #sc-45044 and #sc-45045). Protein concentrations were measured with a bicinchoninic acid assay (G-Biosciences, Saint Louis, Missouri). Fifty micrograms of each lysate were used for bis-tris polyacrylamide gel electrophoresis. Resolved lysates were transferred to nitrocellulose membranes. Membranes were blocked with TBS with 5% bovine serum albumin before incubation with primary antibodies.

The anti-phospho-mixed lineage kinase domain like pseudokinase (pMLKL) antibody was obtained from R&D Systems (Minneapolis, Minnesota, catalog #MAB9187-100, 1:1,000 dilution). Antibodies obtained from Proteintech (Rosemont, Illinois) included anti-MLKL (catalog #66675-1-Ig, 1:5,000 dilution) and anti-β-actin (catalog #66009-1-Ig, 1:50,000 dilution). The anti-long-chain-fatty-acid-Coenzyme A ligase 4 (ACSL4) antibody was obtained from Santa Cruz Biotechnology (Dallas, Texas, catalog #sc-365230, 1:1,000 dilution). Appropriate IR-dye secondary antibodies obtained from Jackson ImmunoResearch Laboratories (West Grove, Pennsylvania) were used (Alexa Fluor® 790-conjugated anti-mouse (catalog #115-655-146, 1:50,000 dilution), Alexa Fluor® 680-conjugated anti-mouse (catalog #115-625-146, 1:50,000 dilution), and Alexa Fluor® 680-conjugated anti-rabbit (catalog #115-625-144, 1:50,000 dilution). Band densities were normalized to β-actin, the endogenous control. Twelve replicates were used for statistical analyses.

### Cell Cycle Analyses.

Cells were treated with LCMT-li, radiotherapy or both as described for Annexin V assays. After 24 hours, cells were labeled with 50 μM bromodeoxyuridine (BrdU, BioGems, Westlake Village, California, catalog #5911439) for 4 hours. Cells were lifted with 0.25% trypsin, 2.21 mM ethylenediaminetetraacetic acid (EDTA) (Corning catalog #25-053-CI) after 4 hours and fixed with 70% ethanol overnight at −20° C. Cells were pelleted and permeabilized with 1 M HCl with 0.1% Triton X-100 for one minute. Cells were pelleted and cellular DNA was denatured with 2 M HCl. After neutralization with 1 M Tris, pH 8.5, cells were labeled with FITC-anti-BrdU (Biolegend, catalog #364104, 5 μL/100 μL cell suspension) and 7-aminoactinomycin-D (7-AAD, Biolegend, catalog #420404, 1 μL/100 μL cell suspension). Cells were washed with PBS with 10% FBS, and resuspended in PBS with 0.1% Triton X-100. Cells were analyzed with flow cytometry. Cell cycle analyses were performed twice with 12 replicates, 104 cells/replicate.

### DDR Analyses.

Cells were treated with LCMT-li, radiotherapy or both as described for Annexin V assays. After 24 hours, cells were lifted with 0.25% trypsin, 2.21 mM EDTA, stained with Ghost Dye™ 710 and fixed with formaldehyde as described. Cells were washed with PBS with 1% BSA, and permeabilized with PBS with 0.1% Triton X-100 on ice for 10 minutes. Cells were pelleted and washed twice with PBS with 0.02% Triton X-100. Cells were resuspended in PBS with 1% BSA and 0.3% Tween-20, and stained with FITC-anti-activated-γ-H2AX (Biolegend, catalog #613404). Cells were pelleted and washed with PBS with 1% BSA and 0.1% sodium azide. DDR analyses were performed twice with 12 replicates, 104 cells/replicate.

Statistical analyses were performed with GraphPad Prism software (version 10.4.1). Effective concentrations that inhibited 50% of cells (EC50, log agonist versus response) were estimated with nonlinear fit curves. Absorbance values for cytotoxity assays and relative densities for Western blot assays were compared using Kruskal-Wallis analyses of variance with Dunn's correction for multiple comparisons. For flow cytometric analyses, two-way analyses of variance with Tukey's correction for multiple comparisons were used.

The results of example experiments are now described.

Lcmt-1 Inhibitors have Drug-Like Properties.

This study identified two potential LCMT-1 inhibitors through a small molecule library screening (Table 2). Both compounds were assessed for absorption, distribution, metabolism and excretion (ADME) properties both in silico and in vitro by the laboratory of Dr. Wayne Childers in the Moulder Center for Drug Discovery Research at the Temple University School of Pharmacy. Both compounds were screened using the free online tool SwissADME (Daina, A. et al., 2017, Sci Rep 7: 42717). Both compounds possess many drug-like properties such as molecular weights <500 Daltons, log (partition coefficient (log P)) values <5 and topological surface area (TPSA) values <90. SwissADME predicted that both compounds should demonstrate good cellular permeability and reasonable bioavailability when given either orally or intraperitoneally. No Lipinski (Lipinski, C. A. et al., 2001, Adv Drug Deliv Rev 46(1-3): 3-26); Veber, D. F. et al., 2002, J Med Chem 45(12): 2615-2623) or Brenk (Brenk, R. et al., 2008, ChemMedChem 3(3): 435-444) violations were identified. Neither compound is predicted to be a substrate for the efflux transporter p-glycoprotein or identified as a pan-interference assay structure (Baell, J. B. and G. A. Holloway, 2010, J Med Chem 53(7): 2719-2740).

In vitro data confirmed both compounds to possess reasonable physicochemical/ADME properties for tool molecules. Kinetic solubility in 2% dimethylsulfoxide/PBS was found to be 18.5 μM for LCMT-li and 27.1 μM for N-(2-Hydroxyphenyl)-2-(1-naphthal enyloxy)acetamide, which falls into the “moderately soluble” category. There is no cause for concern at this stage since there are multiple strategies and reagents for enhancing aqueous solubility for animal in vivo studies (Savjani, K. T., A. K. Gajjar and J. K. Savjani, 2012, ISRN Pharm 2012: 195727.). Mouse and human liver microsomal stability studies (predictors of hepatic P450-mediated oxidative metabolism) showed both compounds to have half-life values in the 17-20 minute range in mouse liver microsomes, which is again considered moderate. As expected, both compounds were more stable to oxidative metabolism in human liver microsomes. Surprisingly, both compounds appeared to be stable in the presence of mouse plasma despite possessing groups (amides, esters) that can be susceptible to hydrolytic metabolism. Due to the presence of multiple aryl rings, both compounds possessed somewhat higher binding to plasma proteins. While target tissue exposure is dependent on free, unbound fractions in the plasma, plasma protein binding can protect compounds from hepatic metabolism. Thus, in vivo metabolism and excretion are often determined by a balance between these two properties. In vivo half-life is a complicated property impacted upon by multiple factors and is difficult to predict. However, it is known that compounds with the microsomal and plasma stability seen for these compounds combined with the plasma protein binding data can demonstrate in vivo half lives in the range of 3-5 hours. While these numbers are somewhat low for a drug candidate, they are acceptable for proof of concept tool molecules which can be given multiple times per day and at higher doses to saturate metabolism.

TABLE 3
ADME Properties of LCMT-1 Inhibitors

| | | |
|---|---|---|
| Chemical Symbols | 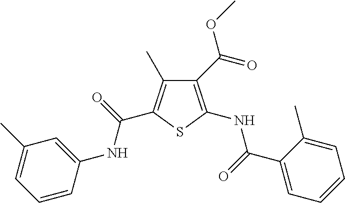 [SMILES: COC(=O)c1c(NC(=O)c2ccccc2C)sc(C(=O)Nc2cccc(C)c2)c1C] | 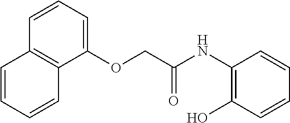 [SMILES: O=C(COc1cccc2ccccc12)Nc1ccccc1O] |
| Chemical Name | 3-Thiophenecarboxylic acid, 4- | N-(2-Hydroxyphenyl)-2- |
| | methyl-2-1(2- | (1-naphthal |
| | methylbenzoyl)amino]-5-[[(3- | enyloxy)acetamide |
| | methylphenyl)amino]carbonyl]-, | |
| | methyl ester (referred to as | |
| | LCMT-1i in this document) | |
| Molecular Weight | 422.50 | 293.32 |
| Max. Kinetic | 18.5 μM | 27.1 μM |
| Solubility | | |
| logP | 3.83 | 2.95 |
| TPSA* | 84.5 | 58.6 |
| Liver Microsomal | | |
| Stability (t_{1/2}) | | |
| Mouse | 17.3 min | 20.0 min |
| Human | 31.1 min | 45.7 min |
| Plasma Protein | 97.1% | 96.7% |
| Binding | | |
| (bound fraction) | | |
| Plasma Stability | >180 min | >180 min |
| (t_{1/2}) | | |

### LCMT-li Sensitized Most Cancer Cell Lines to Irradiation.

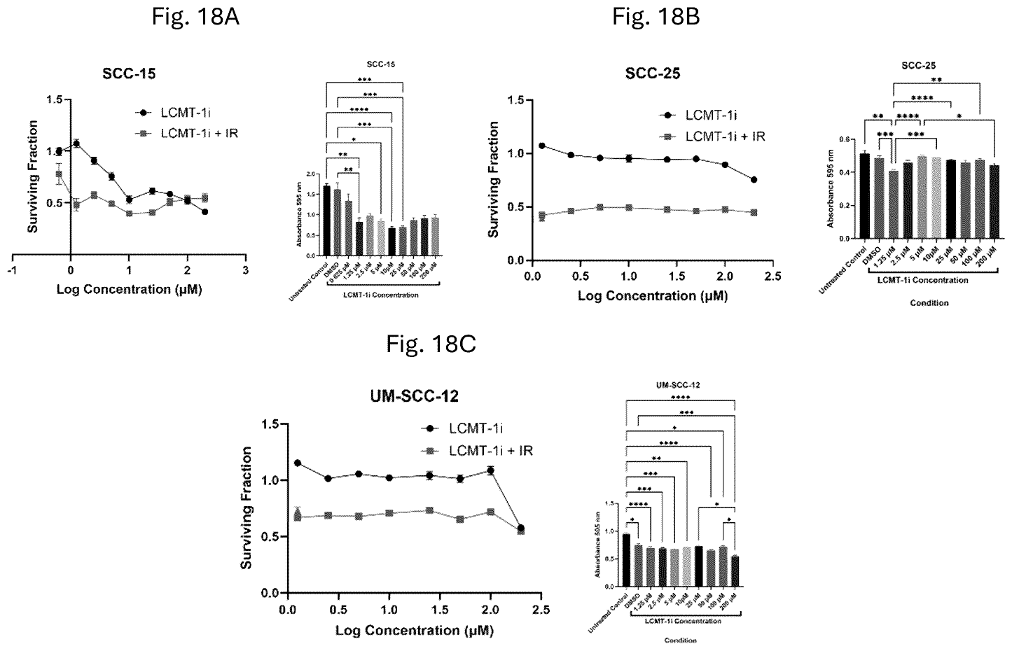

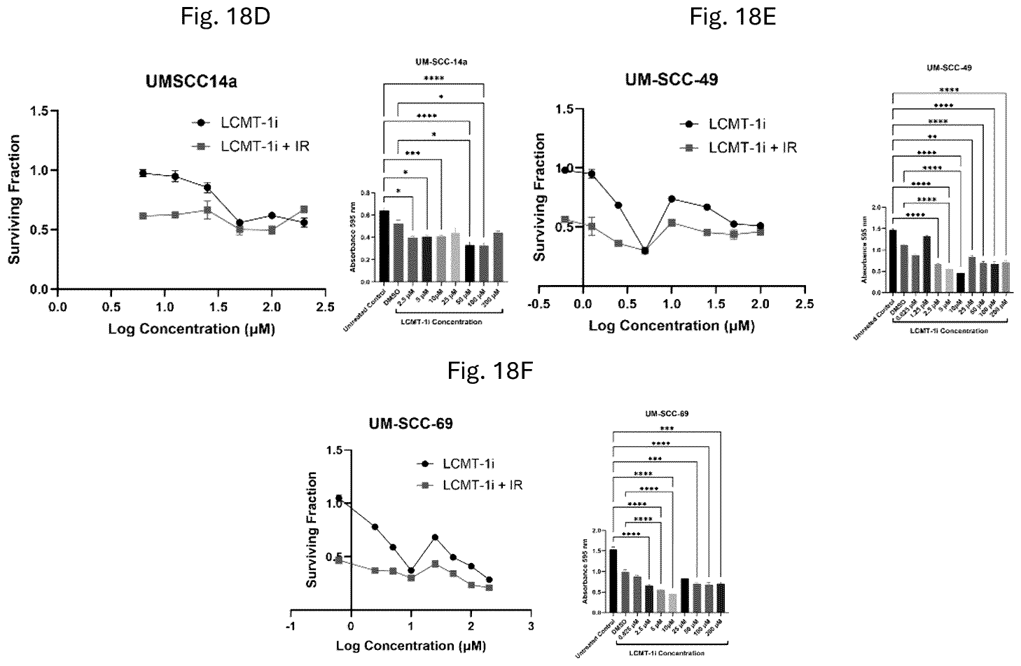

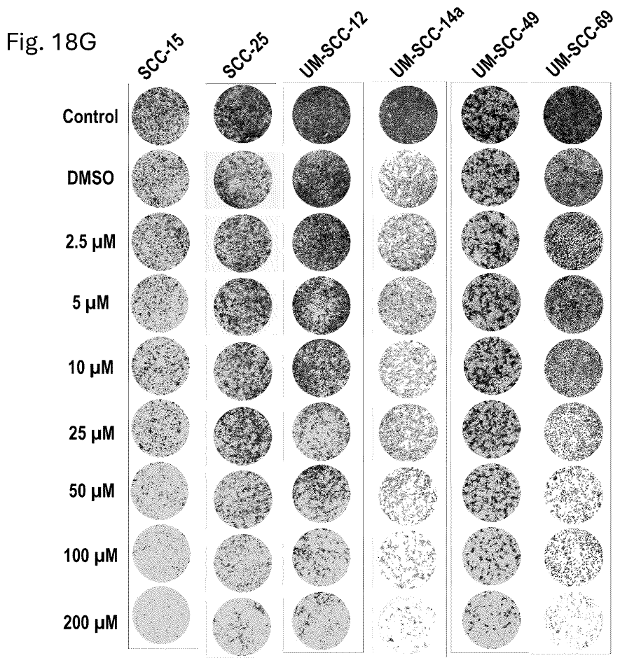

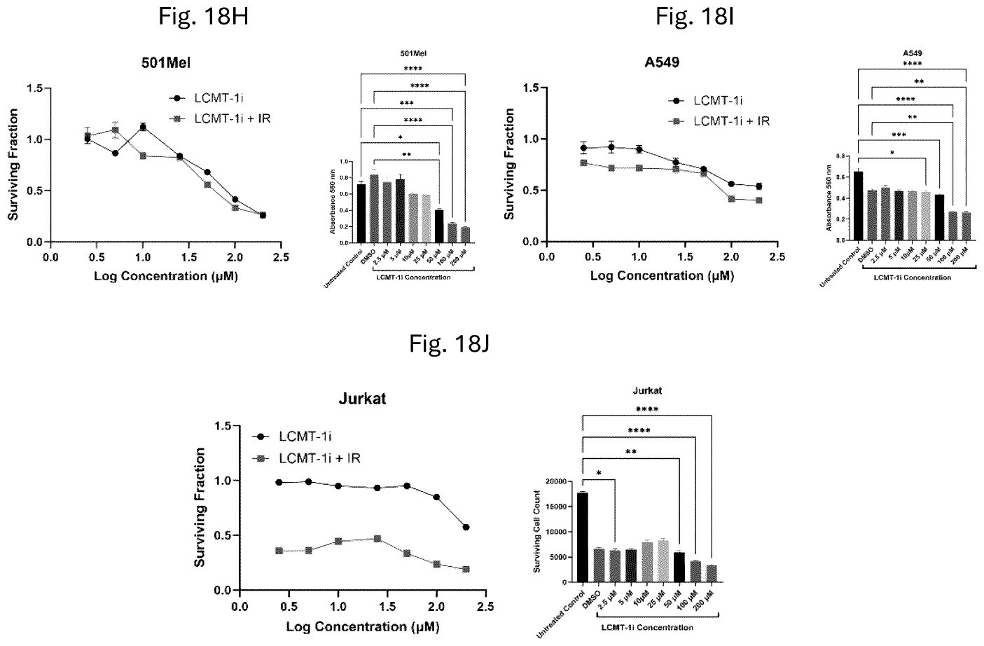

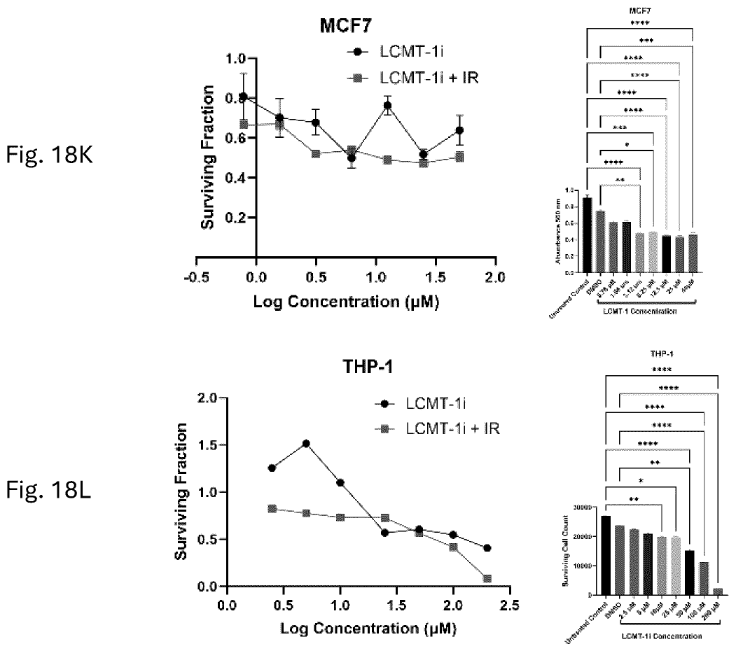

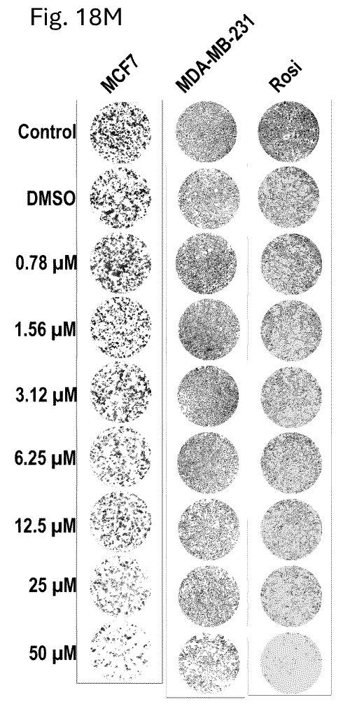

In no cell lines was the radiation dose alone sufficient to significantly inhibit cell survival (FIG. 18). In the HNC cell lines, LCMT-li significantly sensitized all the HNC cell lines to irradiation (FIG. 18A-G, Table 5). While 501MEL human melanoma cells demonstrated radio-resistance, LCMT-li did radiosensitize these cells (FIG. 18H). Of the human leukemia cell lines, Jurkat cells were radiosensitive and did not demonstrate radiosensitization with LCMT-li (FIG. 18J), while THP-1 cells were less radiosensitive and demonstrated LCMT-li radiosensitization (FIG. 18L). Both MCF7 and MDA-MB-231 are triple negative human breast carcinoma cell lines that

TABLE 4
LCMT-1i EC₅₀s

| Cell Line | EC₅₀ (μM) |
|---|---|
| 501-MEL | 60.61 |
| A549 | 41.39 |
| Jurkat | 224.40 |
| MCF7 | 2.24 |
| MDA-MB-231 | 3.89 |
| Rosi | 3.16 |
| SCC-15 | 4.26 |
| SCC-25 | 300,650 |
| THP-1 | 10.42 |
| UM-SCC-12 | 208.9 |
| UM-SCC-14a | 26.36 |
| UM-SCC-49 | 4.95 |
| UM-SCC-69 | 2.49 |

demonstrated inhibition with LCMT-li (Arosarena O. A. et al., 2024, Frontiers in Drug Discovery 4). However, LCMT-li did not radiosensitize MDA-MB-231 cells (data not shown), but did sensitize MCF7 cells to irradiation (FIGS. 18K and 18M). Rosi human melanoma cells (FIG. 18M and Table 5) and A549 human lung carcinoma cells (FIG. 18I) also demonstrated radiosensitization with LCMT-li. Radiosensitization doses did not seem to correlate with EC₅₀ doses.

TABLE 5
LCMT-1i radiosensitizing concentrations by cell line.

| | LCMT-1i Concentration | |
|---|---|---|
| Cell Line | (μM) | p Value |
| 501MEL | 50 | 0.0012 |
| | 100 | <0.0001 |
| | 200 | <0.0001 |
| A549 | 100 | 0.0031 |
| | 200 | 0.0014 |
| MCF7 | 3.12 | 0.0021 |
| | 6.25 | 0.0148 |
| | 12.5 | <0.0001 |
| | 25 | <0.0001 |
| | 50 | 0.0001 |
| Rosi | 50 | 0.0008 |
| SCC-15 | 1.25 | 0.0083 |
| | 10 | 0.0003 |
| | 25 | 0.0007 |
| SCC-25 | 1.25 | <0.0001 |
| THP-1 | 50 | 0.0026 |
| | 100 | <0.0001 |
| | 200 | <0.0001 |
| UM-SCC-12 | 200 | 0.0001 |
| UM-SCC-14a | 50 | 0.0183 |
| | 100 | 0.0115 |
| UM-SCC-49 | 5 | <0.0001 |
| | 10 | <0.0001 |
| UM-SCC-69 | 5 | <0.0001 |
| | 10 | <0.0001 |

### LCMT-li Potentiates Programmed Cell Death in Some HNC Cell Lines.

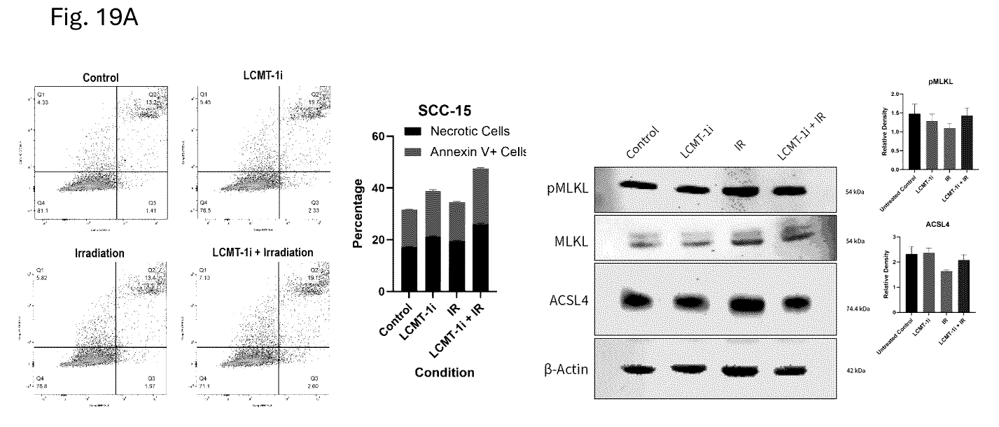

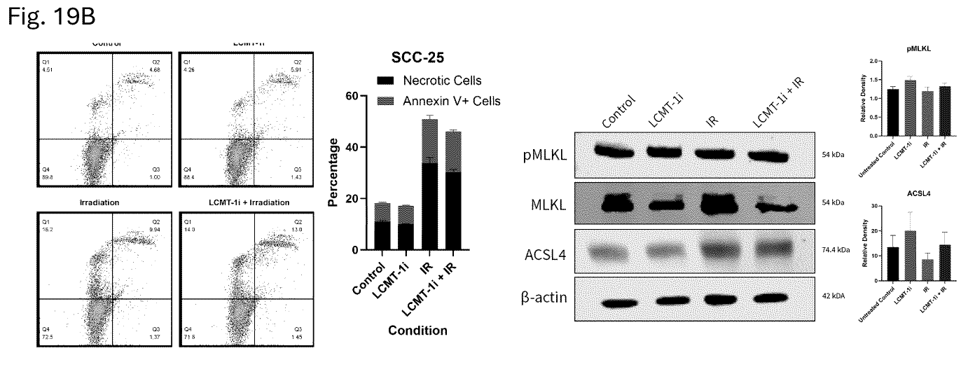

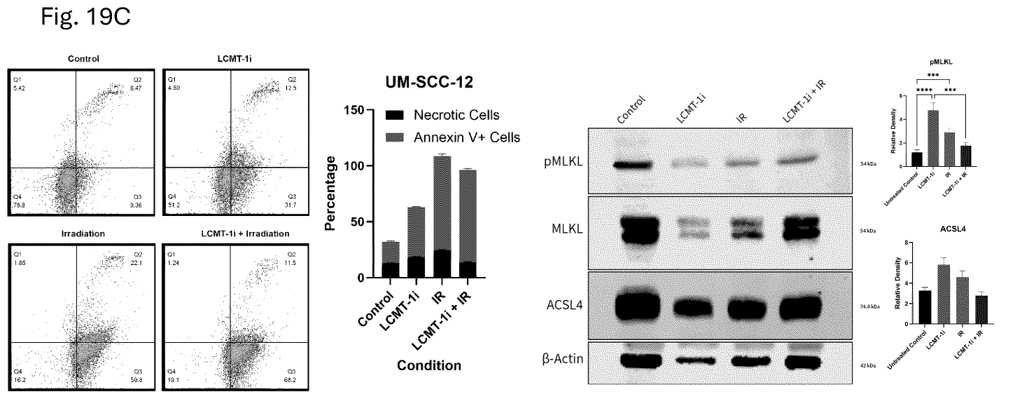

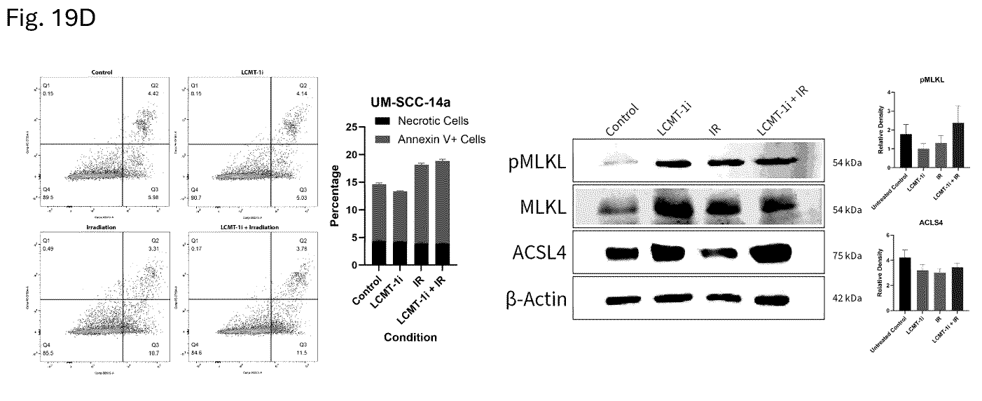

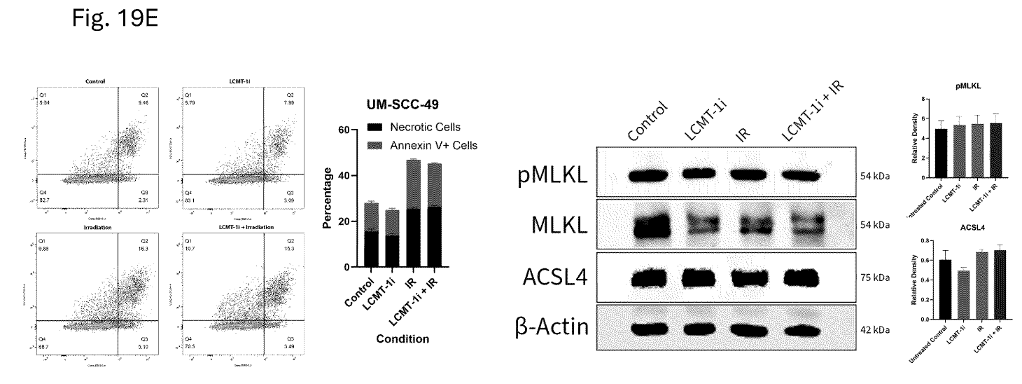

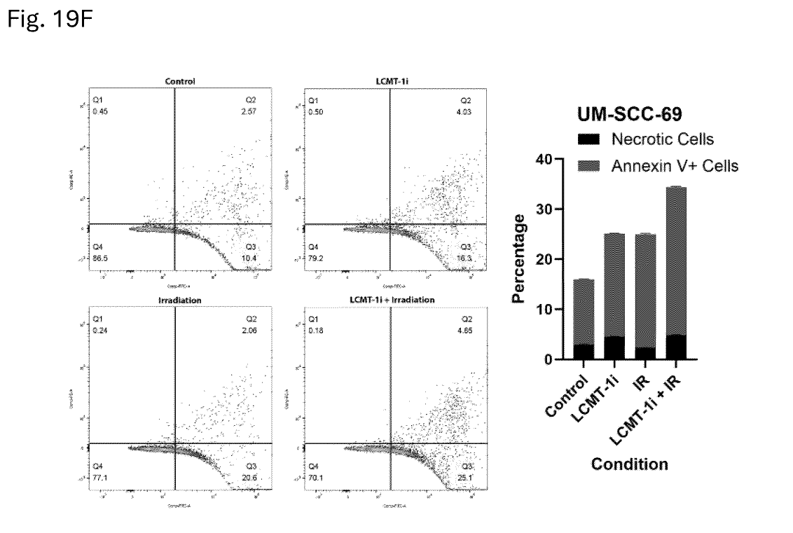

To determine if LCMT-li radiosensitization induced programmed cell death in HNC cell lines, cells were tested for markers of apoptosis, necroptosis and ferroptosis. LCMT-li monotherapy increased apoptosis in SCC-15, UM-SCC-12, and UM-SCC-69 cells (p<0.0001 for all comparisons, FIG. 19) in comparison to control cells. LCMT-li with irradiation increased apoptosis in comparison to irradiation alone in SCC-15 and UM-SCC-69 cells (p<0.0001 for both comparisons). SCC-25 and UM-SCC-49 cells responded to irradiation primarily with necrosis. LCMT-li increased necroptosis in SCC-25 cells as evidenced by increased pMLKL expression. LCMT-li alone increased necroptosis and ferroptosis in UM-SCC-12 cells.

LCMT-li Combined with Irradiation has Variable Effects on the Cell Cycle.

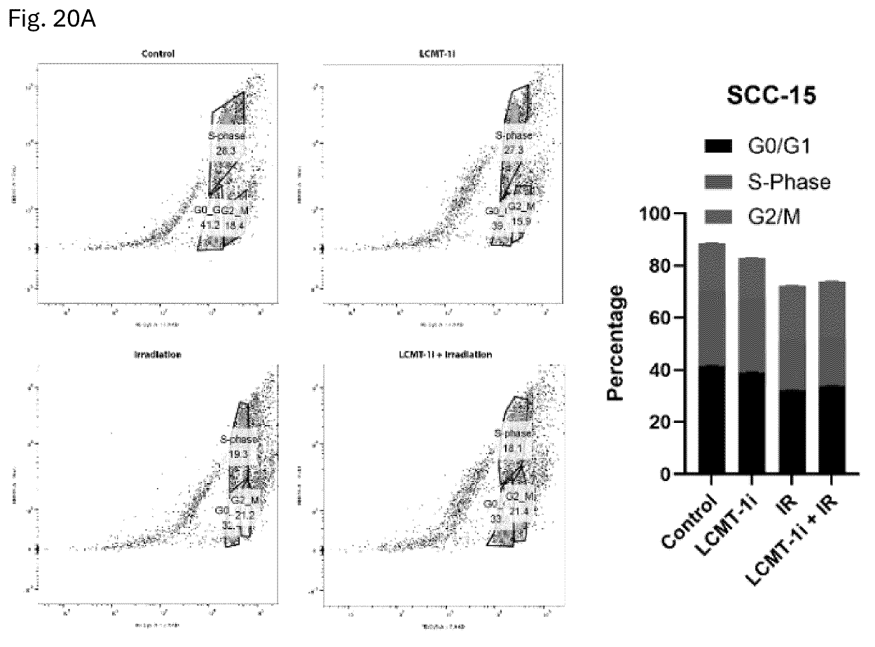

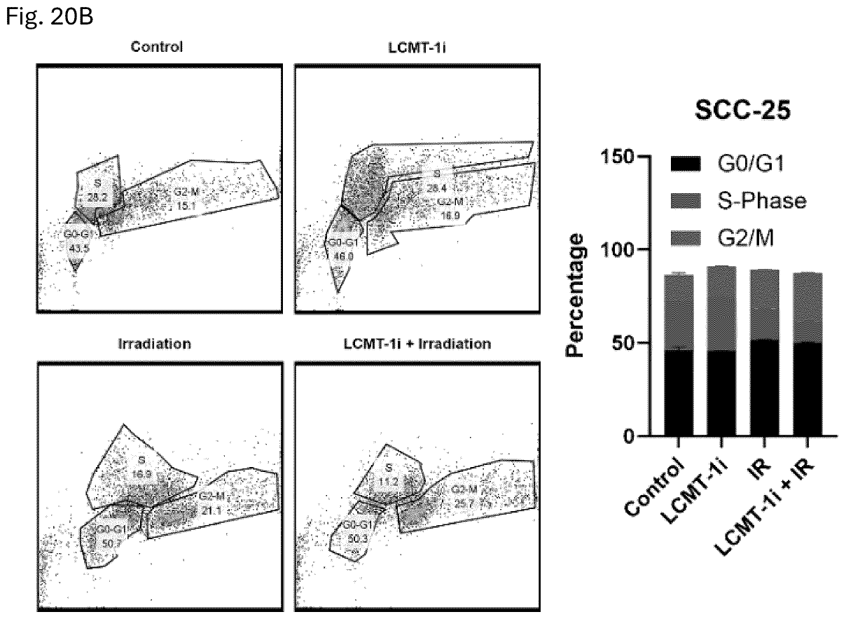

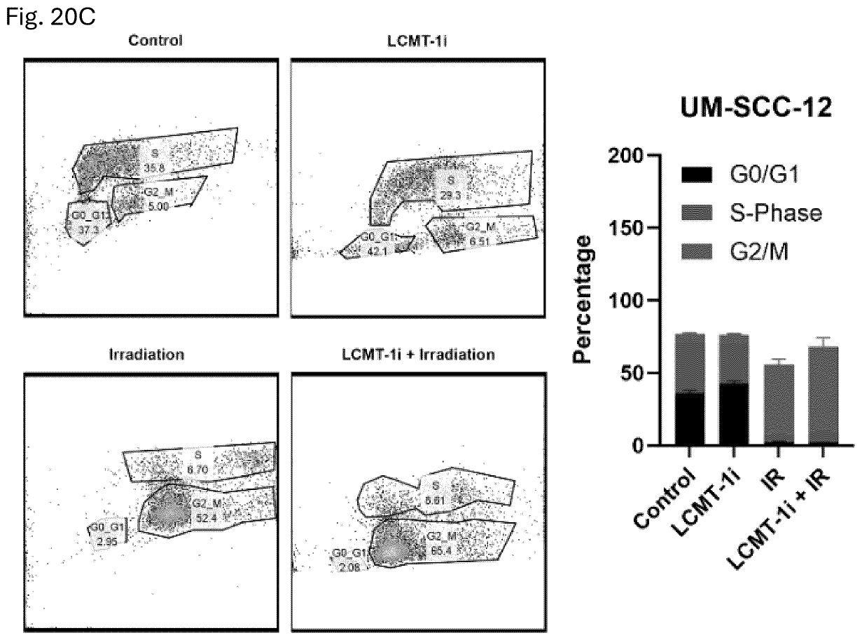

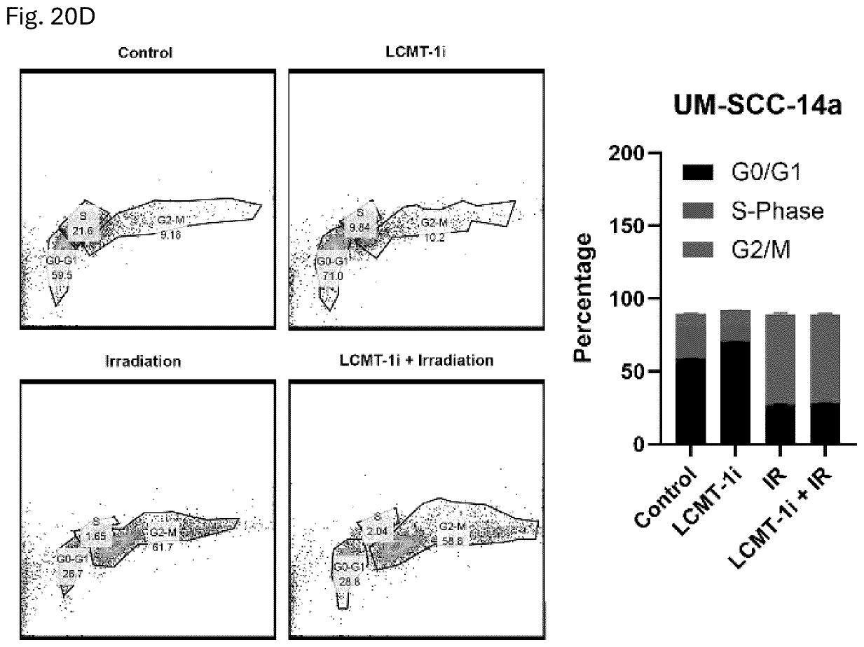

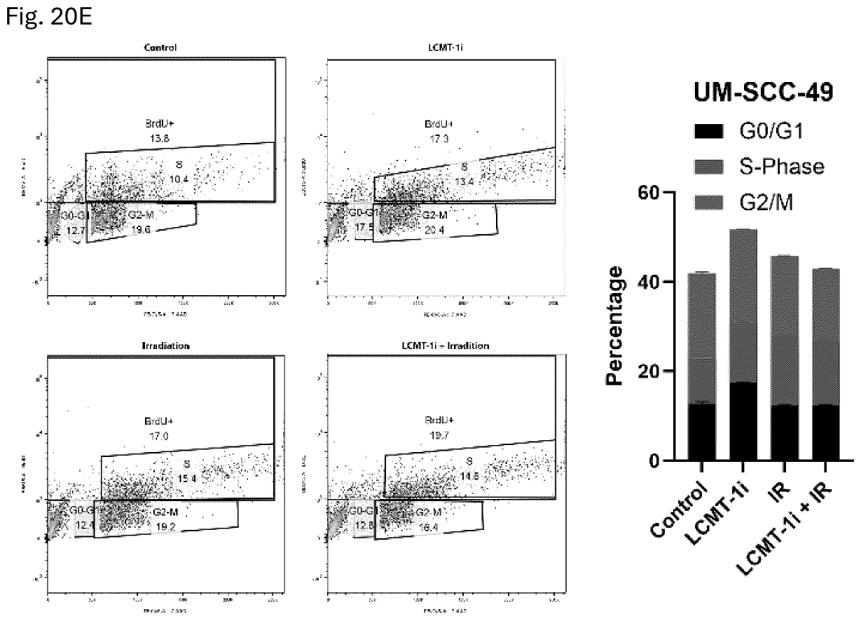

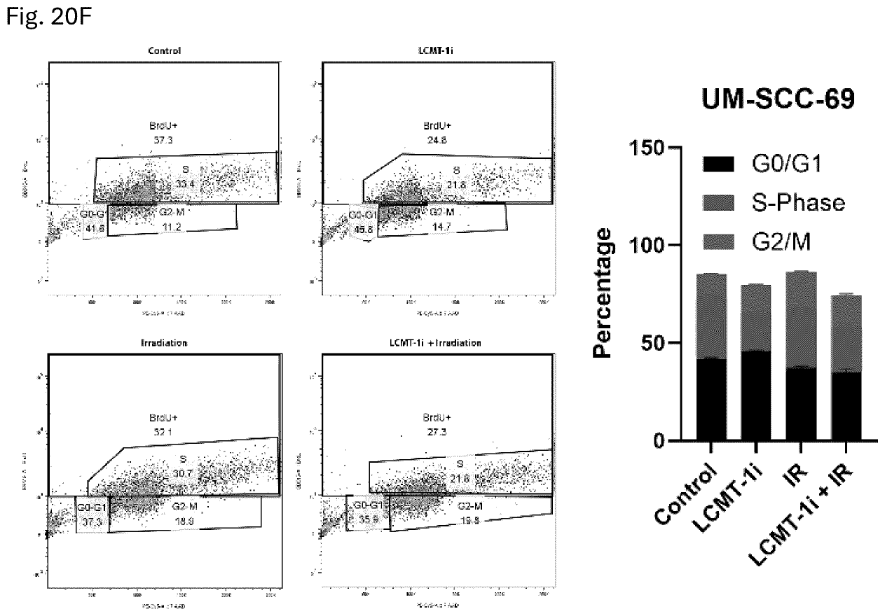

To determine LCMT-li potentiation of irradiation was due to inhibition of mitosis, cell cycle changes were assessed. LCMT-li alone induced G0/G1 arrest in UM-SCC-14a, UM-SCC-49 and UM-SCC-69 (p<0.0001 for UM-SCC-14a and UM-SCC-49 cells, p=0.0003 for UM-SCC-69 cells, FIG. 20) as compared to control cells. LCMT-li alone induced G2/M phase arrest in SCC-25, UM-SCC-14a, and UM-SCC-49 cells (p<0.0001 for SCC-25, p=0.016 for UM-SCC-14a, p=0.0001 for UM-SCC-49). In the presence of irradiation, LCMT-li induced G0/G1 as well as G2/M phase arrest in SCC-15 cells (p<0.0001 for both comparisons). SCC-25 and UM-SCC-12 cells demonstrated increased G2/M phase arrest with the LCMT-li/irradiation combination as compared to irradiation alone (p<0.0001 for SCC25, p=0.0204 for UM-SCC-12). Combination treatment increased G0/G1 arrest in UM-SCC-14a cells as compared to irradiation alone (p=0.0233).

### LCMT-li Inhibits DDR in HNC Cell Lines.

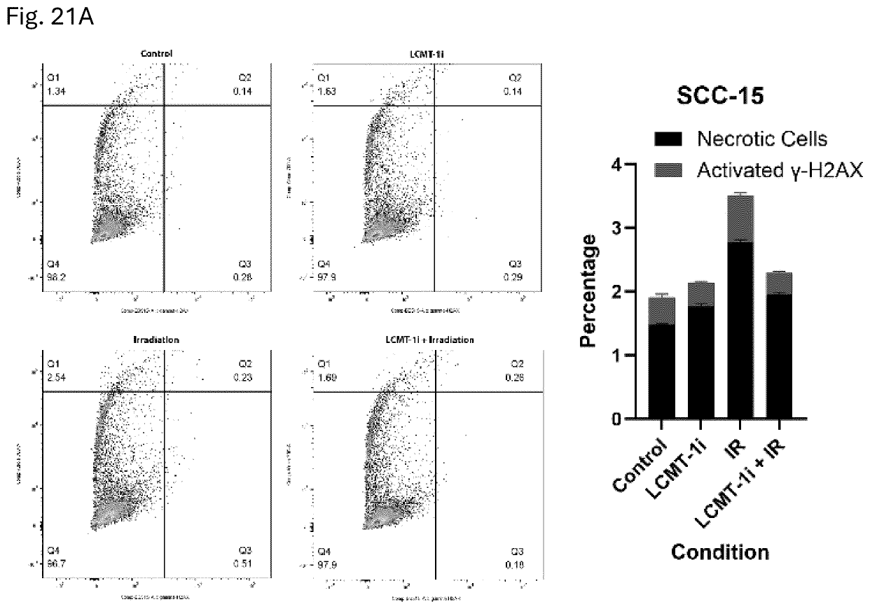

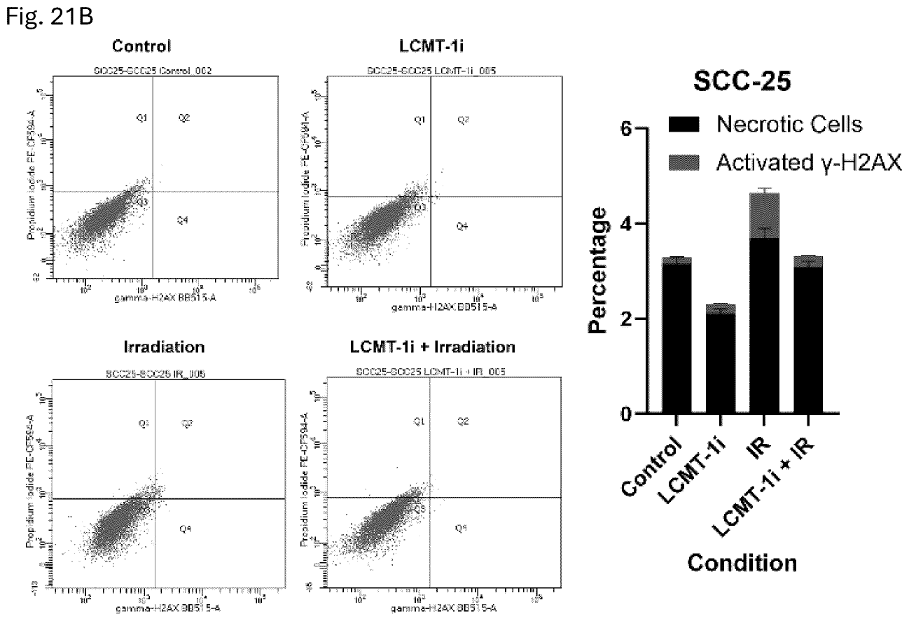

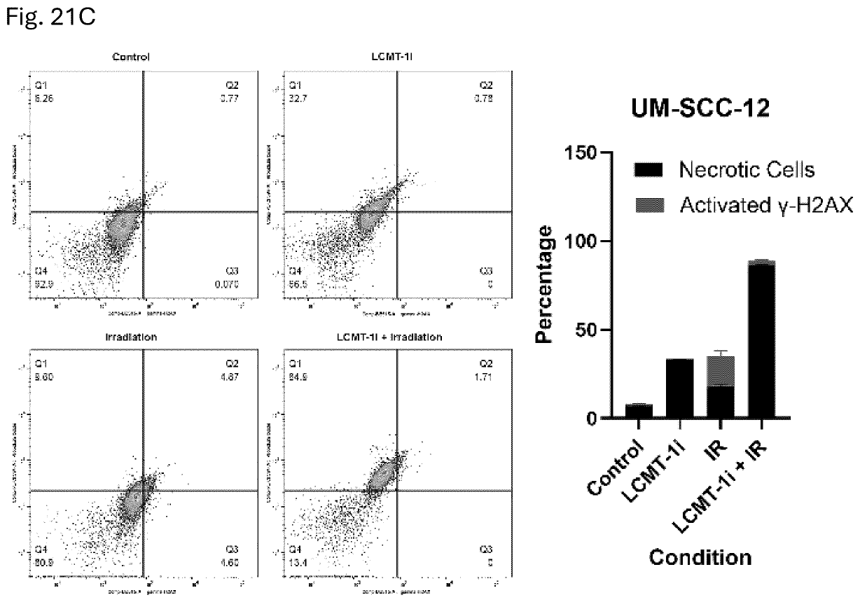

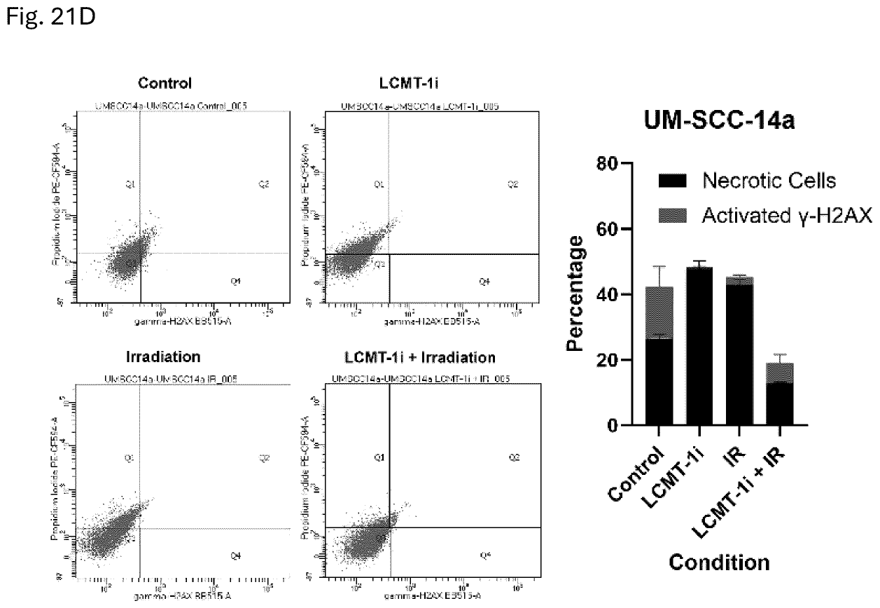

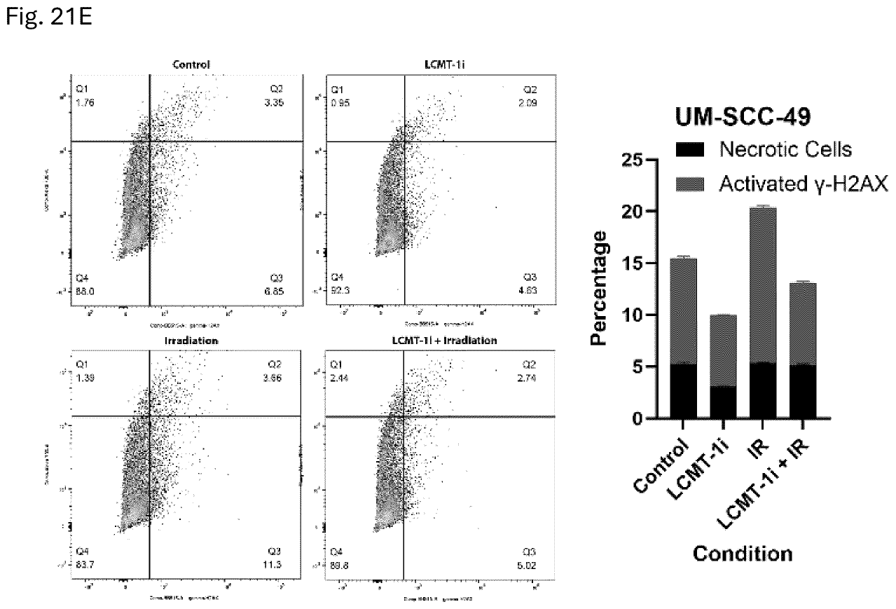

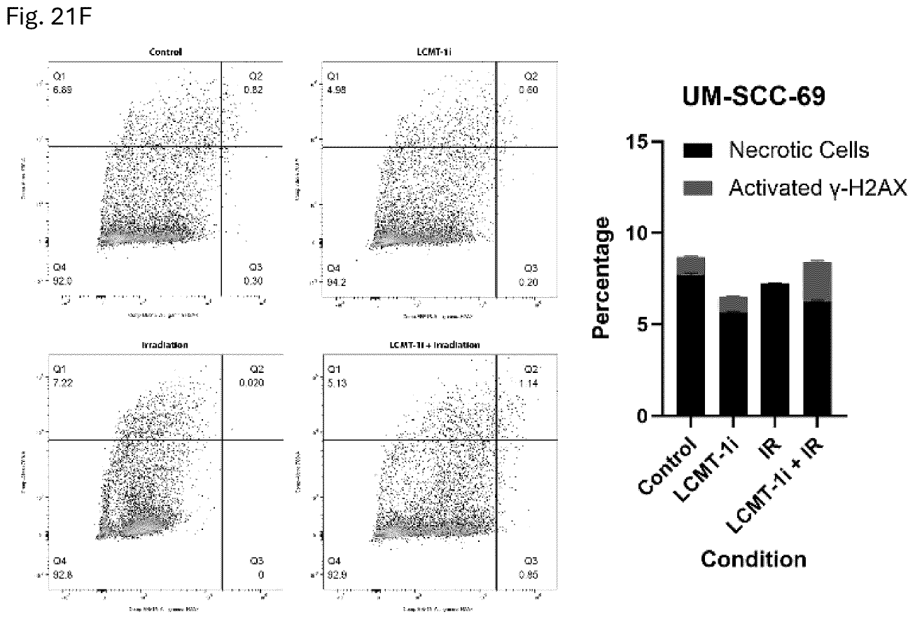

While cell cycle arrest is felt to be cytostatic (Ruiz-Losada, Gonzalez et al. 2022), inhibition of the DDR can result in synthetic lethality (Cheng, Pan et al. 2022). Activated γ-H2AX is a marker of DNA damage induced by ionizing radiation and its expression peaks 30 minutes after radiation exposure (Redon, Dickey et al. 2009). Prolonged expression of activated γ-H2AX is indicative of failure to repair DNA double-strand breaks (Bourton, E. C. et al., Int J Cancer 129(12): 2928-2934) as well as of G2/M phase arrest following exposure to ionizing radiation (Tu, W. Z. et al., 2013, FEBS Lett 587(21): 3437-3443). UM-SCC-14a and UM-SCC-49 cells expressed relatively high baseline activated γ-H2AX characteristic of cells with impaired DDR and high mutational burden ((Ji, J.et al., 2017, PLoS One 12(2): e0171582) FIG. 21). LCMT-li alone increased activated γ-H2AX expression in SCC-25 as compared to control cells (p=0.0001); and decreased activated γ-H2AX expression in UM-SCC-12, UM-SCC-14a, UM-SCC-49 and UM-SCC-69 cells (p<0.0001 for all comparisons). In the presence of irradiation, LCMT-li only inhibited DDR as evidenced by persistent activated γ-H2AX expression in UM-SCC-69 cells (p<0.0001).

LCMT-li has drug-like properties and sensitized all the cell lines tested, except for Jurkat human leukemia cells and MDA-MB-231 human breast carcinoma cells, to ionizing radiation. Cytotoxicity in the six human HNC cell lines tested was mediated primarily through apoptosis, though necroptosis was demonstrated in UM-SCC-12 cells. Ferroptosis was not seen in the HNC cell lines treated with LCMT-li and/or irradiation. Ferroptosis has been shown to be inhibited by cell cycle arrest (Lee, H. et al, 2024, Nat Commun 15(1): 79). LCMT-li alone induced cell cycle arrest in all the HNC cell lines except UM-SCC-12. In the presence of radiation, LCMT-li enhanced cell cycle arrest in SCC-15, SCC-25, UM-SCC-12 and UM-SCC-14a cells. LCMT-li alone decreased activated γ-H2AX expression in the majority of HNC cell lines, but increased activated γ-H2AX expression in SCC-25 cells. In the presence of irradiation, LCMT-li only inhibited DDR as evidenced by persistent activated γ-H2AX expression in UM-SCC-69 cells. These data indicate that LCMT-li can sensitize some cancer cells to irradiation through inducing cell cycle arrest, cell death and inhibition of the DDR.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of Formula (I), or a derivative, prodrug, or pharmaceutically acceptable salt or solvate thereof: 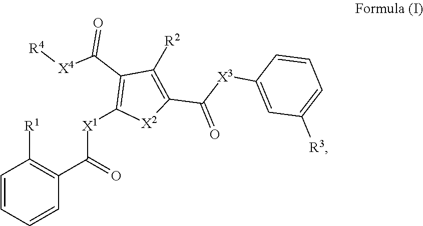
wherein:
R¹, R², R³, and R⁴ are each independently selected from the group consisting of CH₃, CD₃, and CF₃;
X¹, X², X³, and X⁴ are each independently selected from the group consisting of O, NR^{X}, and CR^{X}₂; and
each instance of R^{X} is independently selected from the group consisting of H, D, F, CH₃, CD₃, and CF₃.

2. The compound of claim 1, wherein X¹ is NH.

3. The compound of claim 1, wherein X² is S.

4. The compound of claim 1, wherein X³ is NH.

5. The compound of claim 1, wherein X⁴ is O.

6. The compound of claim 1, wherein at least one of R¹, R², R³, and R⁴ is CH₃.

7. The compound of claim 6, wherein R¹, R², R³, and R⁴ are all CH₃.

8. The compound of claim 1, wherein the compound Formula (I) is a compound of Formula (II): 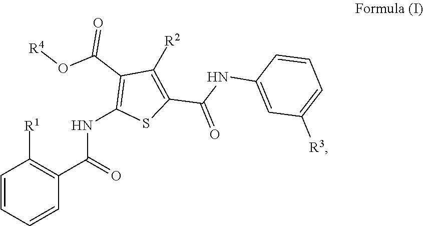
wherein R¹, R², R³, and R⁴ are each independently selected from the group consisting of CH₃, CD₃, and CF₃.

9. The compound of claim 8, wherein at least one of R¹, R², R³, and R⁴ is CH₃.

10. The compound of claim 9, wherein R¹, R², R³, and R⁴ are all CH₃.

11. A method of decreasing leucine carboxyl methyltransferase-1 (LCMT-1) activity in a subject comprising administering to the subject the compound of any one of claims 1-10.

12. A method of treating a disease or disorder in a subject comprising administering to the subject the compound of any one of claims 1-10.

13. The method of claim 12, wherein the disease or disorder is cancer.

14. The method of claim 13, wherein the cancer is selected from the group consisting of squamous cell carcinoma, melanoma, lung, leukemia, colorectal, breast, or pancreatic cancer.

15. The method of 14, wherein the compound of Formula (I) is administered in combination with one or more therapies selected from the group consisting of radiation therapy, surgery, chemotherapy, and immune checkpoint inhibitors.

16. The method of claim 15, wherein the therapy is radiation therapy.

17. The method of claim 12, wherein the method comprises:
a) administering to said subject the compound of any one of claims 1-10; and
b) administering to said subject an effective amount of radiation therapy.

18. The method of claim 17, wherein the radiation therapy is administered by way of a regimen selected from the group consisting of: 4-10 grays.

19. A method of sensitizing a tumor in a subject to treatment comprising administering to the subject the compound of any one of claims 1-10.
